SEQUENCE LISTING (30 sequences)

SEQ ID NO: 1 - DNA, 2250 bp, Pichia pastoris
  CDS (1)..(2250)
atgacaggccgtgtcgaccagaaatctgatcagaaggtgaaggaattgatcgaaaagatcgactccgaatccacttccagagtttttcaggaagaaccagtcacttcgatcttgacacgttacgaaccctatgtcgccccaattatattcacgttgttgtcctttttcactcgtatgtacaaaattgggatcaacaaccacgtcgtttgggatgaagctcacttcggaaagtttggctcctactatctcagacacgagttctaccacgatgtccaccctccgttgggtaagatgttggtcggtctatctggctacattgccggttacaatggctcctgggatttcccctccggtcaagagtaccctgactatattgattacgttaaaatgaggttattcaatgccaccttcagtgccttatgtgtgccattcgcctatttcaccatgaaggagattggatttgatatcaagacaacttggctattcacactgatggtcttgtgtgaaacaagttattgtacgttaggaaaattcatcttgctggattcaatgctgctgctattcactgtgactacggttttcacctttgttaggttccataacgaaaacagtaaaccaggaaactcgttttctcgcaaatggtggaaatggcttctgcttactggtatttccattggtctcacttgttccgtcaaaatggtgggtttatttgtcacagtattagttggaatttacacagttgttgacttatggaataaatttggtgatcaatccatttctcgtaagaaatatgctgctcattggctagctcgtttcatcggcttgattgccatcccaattggcgtttttctattgtcattccgtatccattttgaaatattatccaattctggtaccggtgatgcaaacatgtcttcattgttccaagctaaccttcgtggatcatccgtcggaggaggccccagagatgtgaccactctcaactctaaagtgaccataaagagccaaggtttaggatctggtctgttacattcccacgttcaaacttatcctcaaggttccagccaacaacagattacaacctattctcacaaagatgccaacaatgattgggtgtttcaacttacgagagaagactctcgaaacgctttcaaggaagcccactatgtcgttgatggtatgtctgttcgtctcgttcattcaaacactggtagaaacttacacactcaccaagttgctgctcccgtctcctcatccgaatgggaagtcagttgttatggtaatgaaaccattggagacccgaaagataattggattgttgaaattgtcgaccagtatggtgatgaagataagctgagattgcacccattgacctccagtttccgtttgaaatcggcaactctgggatgctatttgggtacttcgggtgcttcactgcctcaatggggtttcagacaaggtgaagttgtttgttacaaaaatccgttccgtagagataagcgcacctggtggaacatcgaggaccataacaatcctgatctacctaatcctccagaaaattttgttcttcccaggactcattttttgaaagactttgttcaattaaatttagcaatgatggcaacaaacaacgctttggtcccagacccagataaggaagataatctagcttcttctgcctgggaatggcccacgctacacgttggtatccgtctgtgcggttggggcgatgacaacgtcaagtatttcttgattggttctcccgcaaccacctggacttcttcagttggtattgtagtattcctgttcctgctgttaatttacttgatcaaatggcaacgtcaatatgtcattttcccatccgtccagactccactagagtcagccgacaccaaaacagttgcattgtttgacaagtctgatagcttcaacgtcttccttatgggaggattatacccgcttctgggatggggtttacattttgctccgtttgtgatcatgtcgcgtgttacctacgttcaccattatcttcctgcattgtactttgccatgattgttttctgctacttggtttctctgttggataagaaactaggccacccagcattaggattactgatctatgtggctctgtattccttggtcattggaacatttatttggctcagccccgttgtgtttggtatggacggtccgaacagaaattacagttacctaaaccttctacctagttggagagtatcagaccca

SEQ ID NO: 2 - protein, 750 aa, Pichia pastoris
MTGRVDQKSDQKVKELIEKIDSESTSRVFQEEPVTSILTRYEPYVAPIIFTLLSFFTRMYKIGINNHVVWDEAHFGKFGSYYLRHEFYHDVHPPLGKMLVGLSGYIAGYNGSWDFPSGQEYPDYIDYVKMRLFNATFSALCVPFAYFTMKEIGFDIKTTWLFTLMVLCETSYCTLGKFILLDSMLLLFTVTTVFTFVRFHNENSKPGNSFSRKWWKWLLLTGISIGLTCSVKMVGLFVTVLVGIYTVVDLWNKFGDQSISRKKYAAHWLARFIGLIAIPIGVFLLSFRIHFEILSNSGTGDANMSSLFQANLRGSSVGGGPRDVTTLNSKVTIKSQGLGSGLLHSHVQTYPQGSSQQQITTYSHKDANNDWVFQLTREDSRNAFKEAHYVVDGMSVRLVHSNTGRNLHTHQVAAPVSSSEWEVSCYGNETIGDPKDNWIVEIVDQYGDEDKLRLHPLTSSFRLKSATLGCYLGTSGASLPQWGFRQGEVVCYKNPFRRDKRTWWNIEDHNNPDLPNPPENFVLPRTHFLKDFVQLNLAMMATNNALVPDPDKEDNLASSAWEWPTLHVGIRLCGWGDDNVKYFLIGSPATTWTSSVGIVVFLFLLLIYLIKWQRQYVIFPSVQTPLESADTKTVALFDKSDSFNVFLMGGLYPLLGWGLHFAPFVIMSRVTYVHHYLPALYFAMIVFCYLVSLLDKKLGHPALGLLIYVALYSLVIGTFIWLSPVVFGMDGPNRNYSYLNLLPSWRVSDP

SEQ ID NO: 3 - DNA, 1143 bp, Pichia pastoris
  CDS (1)..(1143)
atggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggac

SEQ ID NO: 4 - protein, 381 aa, Pichia pastoris
MAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 5 - DNA, 29 bp, Artificial Sequence
  PCR primer PMT2-KO-5UTR-FW2
attgtcaacgaagttgttggagttaagac

SEQ ID NO: 6 - DNA, 32 bp, Artificial Sequence
  PCR primer PMT2-KO-3UTR-RV2
tttctgttcattttctccagaagctatgtctc

SEQ ID NO: 7 - DNA, 25 bp, Artificial Sequence
  PCR primer PpPMT2-A
aagaagcgttgtagctggaagagca

SEQ ID NO: 8 - DNA, 27 bp, Artificial Sequence
  PCR primer PpRPL10-Prom-RV
gagcaaaatcgagaaggtagtgcatca

SEQ ID NO: 9 - DNA, 29 bp, Artificial Sequence
  PCR primer PpPMT2-B
gagtaaaaccaattatccctgggctttag

SEQ ID NO: 10 - DNA, 22 bp, Artificial Sequence
  PCR primer AOX1-TT-FW
aaaactatgtggcaagccaagc

SEQ ID NO: 11 - DNA, 29 bp, Artificial Sequence
  PCR primer PpPMT2-C
acgttaaaatgaggttattcaatgccacc

SEQ ID NO: 12 - DNA, 30 bp, Artificial Sequence
  PCR primer PpPMT2-D
caccggtaccagaattggataatatttcaa

SEQ ID NO: 13 - DNA, 957 bp, Pichia pastoris
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 14 - DNA, 699 bp, Escherichia coli
atggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 15 - DNA, 2875 bp, Pichia pastoris
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 16 - DNA, 997 bp, Pichia pastoris
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 17 - DNA, 2352 bp, Pichia pastoris
  CDS (1)..(2352)
atgacattcttcttattagactgcctagttttgtataatcttacagaaattctagctcaagccctcttacttgttcttcttctatgtcaactgattcctcaatatatgtggttggtggcccgcgaaatgactcctgagatatttggtcaaacctaccaaaggacaccacaccacagtactatagcacaacaatacatggccgcctttgagtacaaaaagggcattcaaagaccctatttttttaccaagccattggtgaaacctataacgctaagcggctttgaaaaaatacaattggctttgtttcttgcgttcacagtggccgtgagattcttcaatattcaataccccaaccaaattgtatttgatgaggtccattttggaaaatatgcccgaaactacatcaatagctcatacttcatggatgtgcaccctcctttagtcaagatgctttacgccgccataggctatttaggtggttacagaggagattttgttttcaacaagattggggataactacattggtaaagagggtgaaaaattggtaccctacgttttgatgcgatcgtttcccgcaatttgtggagtcttgattgttattctttcttactttatccttagatacagcggatgccgacattttattgcactttttggagctttactggtttgtattgaaaactcattggtagctcaatcaagatttattctactagattctccattgcttttattcattgttctcacagtatacagttttgtgagattcagcaatgaaccagaaccttttggcaaaggctggataagatatctatttttcactggtgtgtccttgggactcagtgtcagtagtaaatgggttggaatattcacaattggttggttaggagtcatgactgtaaaccaattgtggtggttaattggagacttaagcgttcccgatcgtgatgtggtaaagcatgtcttgtacagagcgtattttcttattatcctaccagtgatcatttaccttggggtgtttgcaatccattttttggttctccatgaagctagtggcggttcaggtacagtgagtcctagattcaaagccagtttggacggaactgatttttccaatctttatgctaacgtgtcttttggatccaccgtttcgataagacaccttggtacaggagagtttctacactcccacaaccacacatatcctaaatcgcacaaccaacaggtaaccctatacggatacaaagactccaataatcttttcactattgaaaagaaagataagctatctgacaaggaactattcggcgaggtatccttcctccgacacagagatgttataagattatttcacaagaaaacccaaggatatttgcacgtctctgattctagacctccaattagtgagcaagagtacaacaatgaggtcagtattataggagacaaagactatgtccccgatgtcaatgaaaactttgaggtgaagattatcaaagagtacagtgatgaagatgcaaagcatgaggttaaatccatcggaactgtgtttcaattattccataagggtaccaaatgtactctgtttggtcatcgtgtgaagctgccaaaagactggggatttggtcaattggaggtcacttgtatcgagtcgccagtccttaaaaattctctgtggtacattgaagagaatacacacccacttttcaaccaaacatatcctgcaaaagtgaaagtcgaacccttaggattttttggcaagtttcttgagctgcaccaaaaaatgtggaaaacaaatgcaggcttgactgcctctcacaagtatagctctagacccgaagattggcccgttcttgacagaggtgtgaactatttcaaccgatcaggaaggacgatctacttgttaggtaacttgccaatctattggggaattgtatttactatcggagtattcgttgttttcaagcttgttcagctctggaaatggaagccaaaccatgctccaacagtaaccgatgcttcagctaaatatgattcccaatttttcatctactttgtcggttggctattccatttcgctccatcttttttgatggagcgacagctatttctgcaccactacataccatctctatggtttggtatcatatcaatcgctgtgctcagtgaatatgtttgggctaaactgggaaaaatcgtaggattcttctacgttatgacaatattagggctttcgggtttcttcttctactggtatgccccaatcgtttatgggttagagtggaacaaagacacctgtctgggttcgagactattaccaaactgggacatcccttgcgatcaatttcagtag

SEQ ID NO: 18 - protein, 783 aa, Pichia pastoris
MTFFLLDCLVLYNLTEILAQALLLVLLLCQLIPQYMWLVAREMTPEIFGQTYQRTPHHSTIAQQYMAAFEYKKGIQRPYFFTKPLVKPITLSGFEKIQLALFLAFTVAVRFFNIQYPNQIVFDEVHFGKYARNYINSSYFMDVHPPLVKMLYAAIGYLGGYRGDFVFNKIGDNYIGKEGEKLVPYVLMRSFPAICGVLIVILSYFILRYSGCRHFIALFGALLVCIENSLVAQSRFILLDSPLLLFIVLTVYSFVRFSNEPEPFGKGWIRYLFFTGVSLGLSVSSKWVGIFTIGWLGVMTVNQLWWLIGDLSVPDRDVVKHVLYRAYFLIILPVIIYLGVFAIHFLVLHEASGGSGTVSPRFKASLDGTDFSNLYANVSFGSTVSIRHLGTGEFLHSHNHTYPKSHNQQVTLYGYKDSNNLFTIEKKDKLSDKELFGEVSFLRHRDVIRLFHKKTQGYLHVSDSRPPISEQEYNNEVSIIGDKDYVPDVNENFEVKIIKEYSDEDAKHEVKSIGTVFQLFHKGTKCTLFGHRVKLPKDWGFGQLEVTCIESPVLKNSLWYIEENTHPLFNQTYPAKVKVEPLGFFGKFLELHQKMWKTNAGLTASHKYSSRPEDWPVLDRGVNYFNRSGRTIYLLGNLPIYWGIVFTIGVFVVFKLVQLWKWKPNHAPTVTDASAKYDSQFFIYFVGWLFHFAPSFLMERQLFLHHYIPSLWFGIISIAVLSEYVWAKLGKIVGFFYVMTILGLSGFFFYWYAPIVYGLEWNKDTCLGSRLLPNWDIPCDQFQ

SEQ ID NO: 19 - DNA, 27 bp, Artificial Sequence
  Primer PpPMT5-A
tgtcaatcaataagtgtggcaaatgcg

SEQ ID NO: 20 - DNA, 20 bp, Artificial Sequence
  Primer ScCYCTT-RV
gcggatccagcttgcaaatt

SEQ ID NO: 21 - DNA, 29 bp, Artificial Sequence
  Primer PpPMT5-B
ggggaaaatgtacaaggtgtagtatccag

SEQ ID NO: 22 - DNA, 23 bp, Artificial Sequence
  Primer PpURA5-FW
tttcttctgtttcggagctttgg

SEQ ID NO: 23 - DNA, 34 bp, Artificial Sequence
  Primer PpPMT5-C
aggtcagtattataggagacaaagactatgtccc

SEQ ID NO: 24 - DNA, 31 bp, Artificial Sequence
  Primer PpPMT5-D
ccaatagattggcaagttacctaacaagtag

SEQ ID NO: 25 - DNA, 25 bp, Artificial Sequence
  Primer PpPMT2-A
aagaagcgttgtagctggaagagca

SEQ ID NO: 26 - DNA, 36 bp, Artificial Sequence
  Primer PpTEF-TT-RV
gataaatcgatcaaagttacaaacaataacagtaaa

SEQ ID NO: 27 - DNA, 29 bp, Artificial Sequence
  Primer PpPMT2-B
gagtaaaaccaattatccctgggctttag

SEQ ID NO: 28 - DNA, 22 bp, Artificial Sequence
  Primer AOX1-TT-FW
aaaactatgtggcaagccaagc

SEQ ID NO: 29 - DNA, 29 bp, Artificial Sequence
  Primer PpPMT2-C
acgttaaaatgaggttattcaatgccacc

SEQ ID NO: 30 - DNA, 30 bp, Artificial Sequence; identical to SEQ ID NO: 12
  Primer PpPMT2-D

(19) United States
(12) Patent Application Publication
(10) Pub. No.: US 2015/0337274 A1
(43) Pub. Date: Nov. 26, 2015

(54) PMT2-, OCH1-, PMT5- MUTANT CELLS

(71) Applicants: Ming-Tang CHEN, Lebanon, NH (US); Byung-Kwon CHOI, Lebanon, NH (US); Robert DAVIDSON, (US); MERCK SHARP & DOHME CORP., Rahway, NJ (US)

(72) Inventors: Ming-Tang Chen, Bedford, NH (US); Byung-Kwon Choi, Norwich, VT (US); Robert Davidson, Enfield, NH (US)

(21) Appl. No.: 14/652,964

(22) PCT Filed: Dec. 13, 2013
(86) PCT No.: PCT/US13/74845

§ 371 Date: Jun. 17, 2015

Related U.S. Application Data
(60) Provisional application No. 61/737,934, filed on Dec. 17, 2012.

(51) Int. Cl. C12N 9/12; C12P 21/00

(52) U.S. Cl. CPC C12N 9/1241; C12P 21/005

(57) ABSTRACT

The presented invention relates to the gene knockouts of the Pichia pastoris PMT2 gene in the och1-glycoengineered strain backgrounds to obtain recombinant proteins reduced amounts of O-linked glycosylation. Triple mutant, pmt2, pmt5, och1 strains are also part of the present invention. Method for making such strains and for producing heterologous polypeptides in such strains are also included in the present invention.

This application claims the benefit of U.S. Provisional Patent Application No. 61/737,934, filed Dec. 17, 2012; which is herein incorporated by reference in its entirety.

### FIELD OF THE INVENTION

The field of the present invention relates to fungal or lower eukaryotic cells, such as Pichia pastoris, comprising pmt2, och1 or pmt2, och1, pmt5 mutation as well as methods of making such cells and methods of expressing a polypeptide in such a cell.

### BACKGROUND OF THE INVENTION

When mammalian proteins are recombinantly expressed in the methylotrophic yeast Pichia pastoris, abnormal O-mannosylation often occurs, particularly in the case of monoclonal antibodies (mAbs). O-mannosylation is an essential protein modification in eukaryotes (Strahl-Bolsinger et al). It is initiated at the endoplasmic reticulum by Protein-O-mannosyltransferases (Pmt's) that catalyze the addition of mannose residues to serine or threonine residues of target proteins. The PMT family is phylogenetically classified into PMT1, PMT2 and PMT4 subfamilies, which differ in protein substrate specificity and number of genes per subfamily. While there appear to be five PMT genes encoding Pmt homologues in P. pastoris, O-mannosylation of secreted heterologous proteins in P. pastoris is primarily dependent on the gene encoding Pmt2p. Since the structure of yeast O-linked sugar chains differs from that of mammalian cells, it is preferable to have reduced or completely absent yeast O-linked sugar chains on secreted therapeutic proteins. Furthermore, suppression of yeast O-mannosylation has also been associated with increased protein quality and fermentation titer (Kuroda et al.).

In S. cerevisiae, the PMT family is highly redundant, Tanner et al. in U.S. Pat. No. 5,714,377 described the PMT1 and PMT2 genes of S. cerevisiae and a method of making recombinant proteins having reduced O-linked glycosylation by knocking out individual or certain combination of PMTs. Unlike S. cerevisiae, where the PMT2 family consists of three member proteins: PMT2, PMT3, and PMT6, in some other yeasts or fungi, only PMT2 is present in their genome (e.g., S. pombe, C. albicans, A. fumigatus and C. neoformans) (Willger et al). In these organisms, the PMT2 genes are reported to be essential and cannot be deleted. In P. pastoris, the PMT2 gene family consists of the PMT2 and PMT6 genes. P. pastoris does not have PMT3. PpPmt2p and PpPmt6p share a 44.4% amino acid identity. Evidence suggested that, in an N-linked glycoengineered strain background, PMT2 and OCH1 were synthetically lethal and, thus, it was believed to be impossible to achieve pmt2 knockouts in any och1⁻ N-linked glycoengineered strain background.

### SUMMARY OF THE INVENTION

The present invention provides an isolated fungal or lower eukaryotic host cell, e.g., a Pichia cell, wherein said cell does not express functional PMT2 polypeptide as well as an isolated Pichia cell of wherein said cell does not express functional PMT2 polypeptide and does not express functional OCH1 polypeptide, and, optionally, does not express functional PMT5 polypeptide. In an embodiment of the invention, the endogenous chromosomal PMT2, PMT5 and/or OCH1 genes, in such fungal or lower eukaryotic host cells, e.g., Pichia cells, are partially deleted (e.g., wherein part of the gene is replaced with another polynucleotide such as an auxotrophic marker), fully deleted (e.g., wherein all of the gene is replaced with another polynucleotide such as an auxotrophic marker), point mutated (e.g., introducing one or more missense or nonsense mutations) or disrupted (e.g., with an auxotrophic marker). In an embodiment of the invention, the fungal or lower eukaryotic host cell, e.g., Pichia cell, is glycoengineered, e.g., wherein the cell wall has an average N-glycan mannose content of about 3-10 mannose residues per N-glycan on said cell wall. The fungal or lower eukaryotic host cells, e.g., Pichia cells, of the present invention may include heterologous polynucleotides that encode heterologous polypeptides, e.g., immunoglobulin polypeptides. The present invention includes the isolated fungal or lower eukaryotic host cells, e.g., Pichia cells, in any form including, in a liquid culture medium, on a solid culture medium or a lysate of the cells.

The present invention also includes isolated fungal or lower eukaryotic host cells, e.g., Pichia cells (e.g., wherein the Pichia cell has a cell wall with an average N-glycan mannose content of about 3-10 mannose residues per N-glycan on said cell wall), produced by a method for producing an isolated pmt2⁻, och1⁻ or pmt2⁻, och1⁻, pmt5⁻ fungal or lower eukaryotic host cell, e.g., Pichia cell, comprising expressing a site-specific recombinase (e.g., Cre) in an och1⁻ or och1⁻, pmt5⁻ fungal or lower eukaryotic host cell, e.g., Pichia cell; wherein site-specific recombinase target sequences (e.g., Lox) are at the 5′ and 3′ side of the endogenous chromosomal PMT2 in the cell; and wherein, the recombinase, when expressed in the cell, recombines the target sequences such that the PMT2 is deleted from the chromosome. The method itself also forms part of the present invention.

The present invention also includes isolated fungal or lower eukaryotic host cells, e.g., Pichia cells (e.g., wherein the Pichia cell has a cell wall with an average N-glycan mannose content of about 3-10 mannose residues per N-glycan on said cell wall), produced by a method for producing an isolated pmt2⁻ och1⁻ or pmt2⁻, Och1⁻, pmt5⁻ fungal or lower eukaryotic host cell, e.g., Pichia cell, comprising deleting endogenous PMT2 in an och1⁻ or och1⁻, pmt5⁻ fungal or lower eukaryotic host cell, e.g., Pichia cell, comprising PMT2 operably linked to an inducible promoter (e.g., AOX) under conditions whereby the promoter is induced (e.g., in the presence of methanol) and then, optionally, culturing the cell under conditions whereby the promoter is not induced. The method itself also forms part of the present invention.

The isolated fungal or lower eukaryotic host cells, e.g., Pichia cells, of the present invention, in an embodiment of invention, further include one or more of the following characteristics: (i) wherein one or more endogenous beta-mannosyltransferase genes are mutated; (ii) comprising a polynucleotide encoding an alpha-1,2 mannosidase enzyme; (iii) wherein one or more endogenous phosphomannosyl transferases are mutated, disrupted, truncated or partially or fully deleted; (iv) comprising a Leishmania sp. single-subunit oligosaccharyltransferase; (v) wherein endogenous ALG3 is mutated, disrupted, truncated or partially or fully deleted; (vi) comprising a polynucleotide encoding an endomannosidase; (vii) comprising one or more polynucleotides encoding a bifunctional UDP-N-acetylglucosamine-2-epimerase/N-acetylmannosamine kinase, an N-acetylneuraminate-9-phosphate synthase, or a CMP-sialic acid synthase; (viii) wherein endogenous ATT1 gene is mutated, disrupted, truncated or partially or fully deleted; (ix) wherein endogenous OCH1 is mutated, disrupted, truncated or partially or fully deleted; (x) comprising a polynucleotide encoding galactosyltransferase; (xi) comprising a polynucleotide encoding nucleotide sugar transporter; (xii) comprising a polynucleotide encoding sialyltransferase; and/or (xiii) comprising a polynucleotide encoding acetylglucosaminyl transferase.

The present invention also provides a method for producing a heterologous polypeptide (e.g., an immunoglobulin) comprising introducing, into a pmt2⁻, och1⁻ or pmt2⁻, och1⁻, pmt5⁻ fungal or lower eukaryotic host cell (e.g., Pichia cell), a polynucleotide encoding the heterologous polypeptide and culturing the host cell comprising the polynucleotide encoding the heterologous polypeptide under conditions allowing expression of the heterologous polypeptide (e.g., in a bioreactor or fermentor), optionally, further comprising isolating the heterologous polypeptide from the cells and/or culture medium in which the cells are cultured.

### BRIEF DESCRIPTION OF THE FIGURES

FIG. 1 shows a cartoon diagram of Golgi N-glycan maturation in human versus wild type P. pastoris. Green circles, mannose; Blue squares, GlcNAc; yellow circles, galactose; pink diamonds, sialic acid.

FIG. 2 shows a schematic of the conditional allelic replacement strategy used to generate och1⁻, pmt2⁻ mutants and two lineages of exemplified strains in which this procedure was successfully used to generate och1⁻, pmt2⁻ mutant strains.

FIG. 3 shows a map of plasmid pGLY2968, which contains the AOX1-promoter driven allele of the P. pastoris PMT2 gene, as well as the P. pastoris URA5 gene as a selectable marker, and P. pastoris HIS3 flanking regions for integration, where the 5′ flanking region contains the entire HIS3 ORF and is linked to the P. pastoris ALG3 transcriptional terminator to maintain an active HIS3 gene. The plasmid also contains the pUC19 sequence for maintenance in E. coli, which is removed prior to transformation into P. pastoris by linearization using the SfiI restriction enzyme.

FIG. 4 shows a map of plasmid pGLY3642 which contains the pmt2::ARG1 replacement allele with the 5′ and 3′ flanking regions of the P. pastoris PMT2 gene flanking the P. pastoris ARG1 gene with endogenous promoter and terminator along with the pUC19 sequence for maintenance in E. coli, which is removed prior to transformation into P. pastoris by linearization using the SfiI restriction enzyme.

FIG. 5 shows a Coomassie-stained SDS-PAGE gel of protein A purified antibody expressed by clones that were transformed with an anti-CD20 mAb containing plasmid and cultivated in 96 well plates, from parental strains that were genetically engineered to have the endogenous PMT2 gene eliminated by conditional allelic replacement. The mAb H and L chain genes are driven by the P. pastoris GAPDH promoter and clones were induced in the presence of glucose.

FIG. 6A shows a reducing Western blot of supernatant from clones expressing anti-CD20 mAb probed with anti-H+L antibody from och1⁻, Pmt2⁺ and och1⁻, pmt2⁻ (with AOX1-PMT2) strains cultivated in glycerol and methanol with and without PMTi-3 O-glycosylation inhibitor. Heavily O-glycosylated forms are visible in the och1⁻, Pmt2⁺ control strain lanes and are indicated by the black arrow. FIG. 6B shows a Coomassie stained SDS-PAGE of protein A purified anti-CD20 mAb from the same clones under glycerol conditions with and without PMTi under non-reducing conditions.

FIG. 7 shows a plasmid map of pGLY2132 which is a HIS3::NatR knock-in plasmid that is used to knock-in to the P. pastoris HIS3 locus while not disrupting the HIS3 gene using the NatR, nourseothricin-resistance gene, as a selectable marker. This plasmid also contains an empty GAPDH-CYC1 cassette as well as the pUC19 sequence for maintenance in E. coli.

FIG. 8 shows a plasmid map of pGLY579 which is a HIS3::URA5 knock-in plasmid that is used to knock-in to the P. pastoris HIS3 locus while not disrupting the HIS3 gene using the P. pastoris URA5 gene as a lacZ-URA5-lacZ counterselectable blaster (Nett et al, 2005), as a selectable marker. This plasmid also contains an empty GAPDH-CYC1 cassette as well as the pUC19 sequence for maintenance in E. coli.

FIG. 9 shows a plasmid map of pGLY5883 which is a TRP2::ZeoR roll-in plasmid that is used to introduce a sequence into P. pastoris TRP2 locus while duplicating the TRP2 target site by linearizing the plasmid within the TRP2 gene prior to transformation and using the ZeoR, zeocin resistance cassette, as a dominant selectable marker. This plasmid also contains dual AOX1-promoter driven cassettes of both the H chain and L chain genes of a humanized anti-human HER2 immunoglobulin. The plasmid also contains pUC19 sequence for maintenance in E. coli.

FIG. 10 shows a Coomassie-stained SDS-PAGE gel of protein A purified antibody expressed by clones that were transformed with an anti-HER2 mAb containing plasmid and cultivated in 96 well plates, from parental strains that were genetically engineered to have the endogenous PMT2 gene eliminated by conditional allelic replacement (YGLY6890, 6891, and 6892). In parallel, a PMT2 wild type strain (och1⁻) previously engineered to contain the anti-CD20 mAb as a growth control was cultivated (YGLY3920). All strains were cultivated in the absence of PMTi-3 inhibitor. Commercially available purified anti-HER2 mAb was run in parallel in 2 fold dilutions as a standard for a loading control.

FIG. 11 shows a map of plasmid pGLY12503. Plasmid pGLY12503 is an integration vector that targets the PMT2 locus and contains in tandem four nucleic acid regions encoding (1) Lox66, a mutant LoxP, (2) P. pastoris TEF transcription terminator, (3) an arsenic resistance marker (ARS) encoded by the S. cerevisiae ARR3 ORF under the control of the P. pastoris RPL10 promoter and S. cerevisiae CYC1 transcription terminator sequences, (4) A. gossypii TEF promoter, all flanked by the 5′ region of the PMT2 gene (PpPMT2-5′) and PMT2 ORF (PpPMT2-ORF). PpTEF TT is the P. pastoris TEF transcription terminator; PpRPL10 Prom is the P. pastoris RPL10 promoter; ScCYC TT is the S. cerevisiae CYC1 transcription terminator; ScARR3 is the S. cerevisiae ARR3 ORF; AgTEF Prom is the A. gossypii TEF promoter.

FIG. 12 shows a map of plasmid pGLY12534. Plasmid pGLY12534 is an integration vector that targets the PMT2 locus and contains in tandem four nucleic acid regions encoding (1) P. pastoris ALG3 termination sequence, (2) P. pastoris URA5 region, (3) a Cre-recombinase expression cassette encoded by the Cre ORF of P1 Bacteriophage under the control of the P. pastoris AOX1 promoter and the P. pastoris AOX1 transcription terminator sequences, (4) Lox72, a mutant LoxP site, all flanked by the PMT2 ORF (PpPMT2-ORF) and the 3′ region of the PMT2 gene (PpPMT2-3′). PpALG3 TT is the P. pastoris ALG3 termination sequence; PpAOX1 Prom is the P. pastoris AOX1 promoter; PpAOX1 TT is the P. pastoris AOX1 termination sequence.

FIG. 13 shows a schematic of the Cre-LoxP recombination strategy used to generate och1 pmt2 mutants and the exemplified anti-HER2 and human Fc producing strain lineages in which this procedure was successfully used to generate och1⁻ pmt2⁻ mutant strains.

FIG. 14 shows a Coomassie-stained SDS-PAGE gel of protein A purified antibody expressed by clones that were transformed with an anti-HER2 mAb containing plasmid and cultivated in 1 Liter DasGip Fermentors, from parental strains that were genetically engineered to have the endogenous PMT2 gene eliminated by the Cre-LoxP recombination technique (YGLY31670, 31673, and 31674, Lanes 1 to 3). All pmt2⁻ strains were cultivated in the absence of PMTi-4 inhibitor. In parallel, the PMT2 wild type parental strain YGLY27983 (och1⁻) was cultivated without (Lane 4) and with (Lane 5) PMTi-4 inhibitor as controls.

FIG. 15 shows a Coomassie-stained SDS-PAGE gel of protein A purified antibody expressed by clones that were transformed with a human Fc containing plasmid and cultivated in 1 Liter DasGip Fermentors, from parental strains that were genetically engineered to have the endogenous PMT2 gene eliminated by the Cre-LoxP recombination technique (YGLY32116, 32117, 32118, 32121 and 32122, Lanes 3 to 6). All pmt2⁻ strains were cultivated in the absence of PMTi-4 inhibitor. In parallel, the PMT2 wild type parental strain YGLY29128 (och1⁻) was cultivated without PMTi-4 inhibitor as a control (Lanes 1 and 2).

FIG. 16 shows a schematic of the construction of och1⁻, PMT wild-type control yeast strains producing human Fc, anti-HER2 and anti-RSV proteins.

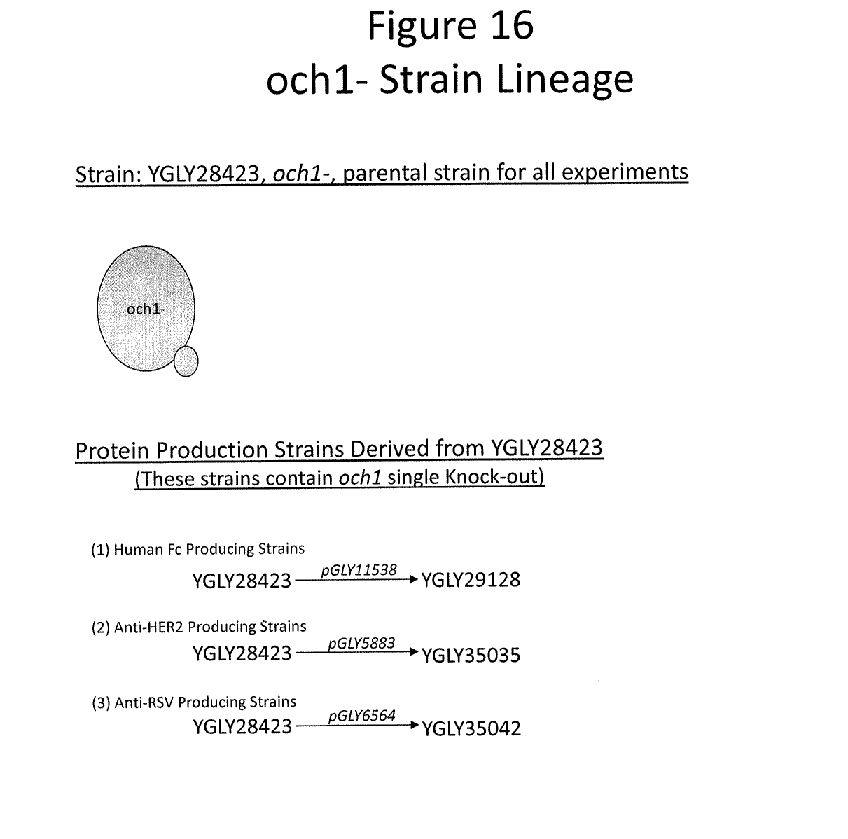

FIG. 17 shows a schematic of using the Cre-LoxP recombination strategy to generate och1, pmt2 double knock-outs mutant strains and the corresponding yeast strains producing human Fc, anti-HER2 and anti-RSV proteins.

FIG. 18 shows a schematic of the Cre-LoxP recombination strategy used to generate och1, pmt2, pmt5 triple KO mutants strains and their corresponding human Fc, anti-HER2 and anti-RSV producing strain lineages

FIG. 19 shows a map of plasmid pGLY12527. Plasmid pGLY12527 is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris PMT5 locus (PpPMT5-5′) and on the other side with the 3′ nucleotide sequence of the P. pastoris PMT5 locus (PpPMT5-3′).

FIG. 20 shows maps of plasmids pGLY12535. Plasmid pGLY12535 is an integration vector that targets the PMT2 locus and contains in tandem five nucleic acid regions encoding (1) PMT2-5′UTR sequences, (2) Lox66, a mutant LoxP site, (3) P. pastoris TEF transcription terminator, (4) A. gossypii TEF transcription promoter, and (5) 5′-end region (amino acid 1 to 226) of a G418-resistance marker (G418-5′-ORF) encoded by an aminoglycoside phosphotransferase of bacterial transposon Tn903.

FIG. 21 shows maps of plasmids pGLY12536. Plasmid pGLY12536 is an integration vector that targets the PMT2 locus and contains in tandem ten nucleic acid regions encoding (1) 3′-end region (amino acid 9 to 269) of a G418-resistance marker (G418-3′-ORF) encoded by an aminoglycoside phosphotransferase of bacterial transposon Tn903, (2) A. gossypii TEF transcription terminator, (3) P. pastoris RPL10 promoter, (4) P. pastoris PMT2 ORF, (5) P. pastoris ALG3 transcription terminator sequences, (6) P. pastoris AOX1 transcription promoter, (7) Cre-recombinase of bacteriophage P1, (8) P. pastoris AOX1 transcription terminator, (9) Lox72, a mutant LoxP site, and (10) the 3′ region of the PMT2 locus (PpPMT2-3′).

FIG. 22 shows a plasmid map of pGLY11538 which is a TRP2::ZeoR roll-in plasmid that is used to introduce a sequence into P. pastoris TRP2 locus while duplicating the TRP2 target site by linearizing the plasmid within the TRP2 gene prior to transformation and using the ZeoR, zeocin resistance cassette, as a dominant selectable marker. This plasmid also contains an AOX1-promoter driven human Fc expression cassette. The plasmid also contains pUC19 sequence for maintenance in E. coli.

FIG. 23 shows a plasmid map of pGLY6564 which is a TRP2::ZeoR roll-in plasmid that is used to introduce a sequence into P. pastoris TRP2 locus while duplicating the TRP2 target site by linearizing the plasmid within the TRP2 gene prior to transformation and using the ZeoR, zeocin resistance cassette, as a dominant selectable marker. This plasmid also contains dual AOX1-promoter driven cassettes of both the H chain and L chain genes of an anti-RSV immunoglobulin. The plasmid also contains pUC19 sequence for maintenance in E. coli.

FIG. 24 shows a Coomassie-stained SDS-PAGE gel of protein-A purified antibody expressed by a clone that was transformed with an anti-HER2 mAb containing plasmid and cultivated in 1 Liter DasGip Fermentor, from a strain that was genetically engineered to have the och1, pmt2, pmt5⁻ triple knock-outs (YGLY35041, Lane 3). In parallel, the och1 anti-HER2 producing strain (YGLY35035, Lane 1) and the och1, pmt2 double knock-outs anti-HER2 producing strain (YGLY35037, Lane 2) was cultivated as controls. All strains were cultivated in the absence of PMTi-4 inhibitor. The Cre-LoxP recombination technique was used to generate pmt2 gene knock-out.

FIG. 25 shows a Coomassie-stained SDS-PAGE gel of protein-A purified antibody expressed by a clone that was transformed with an anti-RSV mAb containing plasmid and cultivated in 1 Liter DasGip Fermentor, from a strain that was genetically engineered to have the och1, pmt2, pmt5 triple knock-outs (YGLY35048, Lane 3). In parallel, the och1 anti-RSV producing strain (YGLY35042, Lane 1) and the och1, pmt2 double knock-outs anti-RSV producing strain (YGLY35044, Lane 2) was cultivated as controls. All strains were cultivated in the absence of PMTi-4 inhibitor. The Cre-LoxP recombination technique was used to generate pmt2 gene knock-out.

### DETAILED DESCRIPTION OF THE INVENTION

The presented invention relates to the generation of gene knockouts of the Pichia pastoris PMT2 gene in an och1⁻ glycoengineered strain background to obtain recombinant proteins with reduced amounts of O-linked glycosylation. Despite an extremely low frequency of occurrence, PMT2 gene knockouts were achieved in och1⁻ glycoengineered Pichia pastoris strains. A pmt2 knockout was not achieved using traditional yeast DNA transformation and recombination methods such as standard one-step double crossover allele integration, and split marker one-step allele integration. The presented invention also provides two separate methods that were used successfully to isolate surviving pmt2⁻ host cells. Both methods, (1) the AOX1 promoter-Pichia pastoris PMT2 inducible promoter conditional allele replacement approach and (2) the Cre-LoxP recombination technique; generated pmt2⁻, och1⁻ double mutants in N- and O-linked glycoengineered strain backgrounds with improved quality and high yields of recombinant protein expression. Knocking out PMT2 resulted in a more than 2-fold fermentation mAb titer improvement as well as better protein folding and assembly relative to PMT2, och1⁻ cells. A benefit of the invention is that, with the pmt2⁻, och1⁻ strains, the requirement of adding certain benzylidene thiazolidinedione inhibitors of Pmt-mediated O-linked glycosylation in cell culture is eliminated.

An isolated fungal or lower eukaryotic host cell, e.g., a Pichia host cell, lacking functional OCH1 polypeptide may be referred to as an och1 or och1⁻ cell. An isolated fungal or lower eukaryotic host cell, e.g., a Pichia host cell, lacking functional PMT5 polypeptide may be referred to as a pmt5⁻ or pmt5⁻ cell. Likewise, an isolated Pichia host cell lacking functional PMT2 polypeptide may be referred to as a pmt2 or pmt2⁻ cell. An isolated Pichia host cell lacking functional PMT2 polypeptide and OCH1 polypeptide may be referred to as a pmt2, och1 or pmt2⁻, och1⁻ cell. An isolated Pichia host cell having functional PMT2 polypeptide and lacking OCH1 polypeptide may be referred to as a PMT2, och1⁻ cell. Lack of a functional polypeptide may be due to genetic mutation of the endogenous gene or its expression control sequences or modification of the host cell that lacks the protein to decrease levels of expression of the polypeptide below wild-type levels, e.g., by RNA interference, anti-sense DNA or RNA or, use of small interfering RNA or an increase in protein degradation in the cell so as to decrease the level of the polypeptide to below wild-type levels.

A “PMT2^{wt}” or “PMT2” fungal or lower eukaryotic host cell comprises a wild-type PMT2 polypeptide.

A “PMT5^{wt}” or “PMT5” fungal or lower eukaryotic host cell comprises a wild-type PMT2 polypeptide.

“PpPMT2” is Pichia pastoris PMT2.

“PpPMT5” is Pichia pastoris PMT5.

A “OCH1^{wt}” or “OCH1” fungal or lower eukaryotic host cell comprises a wild-type OCH1 polypeptide.

“PpOCH1” is Pichia pastoris OCH1.

“Wild type yeast N-glycosylation” is defined has glycosylation having >15 mannose residues per N-linked site on a Man₈ core N-glycan.

“Reduced N-glycan mannose content” is defined as having 3-10 mannose residues per N-linked site.

A heterologous polynucleotide is a polynucleotide that has been introduced into a fungal or lower eukaryotic host cell and that encodes a heterologous polypeptide. For example, a heterologous polynucleotide can encode an immunoglobulin heavy chain and/or an immunoglobulin light chain, e.g., comprising the light or heavy chain variable domain and, optionally, the antibody constant domain, e.g., from an antibody or antigen-binding fragment thereof, e.g., from a fully human antibody, humanized antibody, chimeric antibody, a bispecific antibody, an antigen-binding fragment of an antibody such as a Fab antibody fragment, F(ab)₂ antibody fragment, Fv antibody fragment, single chain Fv antibody fragment or a dsFv antibody fragment. Any such antibody can bind specifically to any epitope such as insulin-like growth factor 1 receptor, VEGF, interleukin-6 (IL6), IL6 receptor, respiratory syncitial virus (RSV), CD20, tumor necrosis factor alpha, receptor activated NF kappa B ligand (RANKL), or the RANKL receptor RANK, IgE, Her2, Her3, or the epidermal growth factor receptor.

An “endogenous” gene is a natural chromosomal copy of the gene. Expression levels of PMT2 and/or OCH1 in a pmt2⁻, och1⁻ fungal or lower eukaryotic host cell may be reduced below wild-type levels (e.g., such that no functional PMT2 polypeptide and/or OCH1 polypeptide is expressed). In an embodiment of the invention, an endogenous PMT2, PMT5 and/or OCH1 gene in an isolated pmt2⁻, och1⁻ or pmt2⁻, och1⁻, pmt5⁻ fungal or lower eukaryotic host cell is mutated by being partially deleted (e.g., wherein part of the endogenous PMT2, PMT5 and/or endogenous OCH1 is replaced with another polynucleotide such as an auxotrophic marker or a drug resistance marker), thus leaving only part of the PMT2, PMT5 or OCH1 coding sequence in the chromosomal locus where PMT2, PMT5 or OCH1 would naturally occur; fully deleted (e.g., wherein all of the endogenous PMT2, PMT5 and/or endogenous OCH1 is replaced with another polynucleotide such as an auxotrophic marker or a drug resistance marker), thus leaving no PMT2, PMT5 or OCH1 coding sequence in the chromosomal locus wherein PMT2, PMT5 or OCH1 would naturally occur; disrupted (e.g., wherein another polynucleotide, such as an auxotrophic marker or a drug resistance marker, is inserted into the endogenous PMT2, PMT5 and/or endogenous OCH1), thus inserting a heterologous sequence into the chromosomal PMT2, PMT5 or OCH1 gene; or point mutated at one or more points in the chromosomal gene (e.g., missense or nonsense mutation). Alternatively, the regulatory region of such an endogenous PMT2, PMT5 or OCH1 gene may be mutated, e.g., partially or fully deleted, disrupted or mutated such that reduced amounts (e.g., no significant amount) of functional PMT2, PMT5 or OCH1 polypeptide are expressed in the cell. In another embodiment of the invention, expression of PMT2, PMT5 and/or OCH1 may be reduced by interference with transcription and/or translation of PMT2, PMT5 and/or OCH1, e.g., by introduction of small interfering RNA, antisense RNA, antisense DNA, RNA interference molecules or by reduction of PMT2, PMT5 and/or OCH1 polypeptide half-life in the cell, for example by modulation of ubiquitination of the polypeptides. Such isolated pmt2⁻, och1⁻ or pmt2⁻, och1⁻, pmt5-fungal or lower eukaryotic host cells, method of making such cells and methods for expressing heterologous polypeptides using such cells (e.g., as discussed herein) are part of the present invention.

Examples of Pmt inhibitors (PMTi) include but are not limited to a benzylidene thiazolidinediones such as those disclosed in U.S. Pat. No. 7,105,554 and U.S. Published Application No. 20110076721. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; 5-[[3-(1-Phenyl-2-hydroxy)ethoxy)-4-(2-phenylethoxy)]phenyl]methylene]-4-oxo-2-thioxo-3-thiazolidineacetic Acid; and, Example 4 compound in U.S. Published Application No. US2011/0076721).

### Molecular Biology

In accordance with the present invention there may be employed conventional molecular biology, microbiology, and recombinant DNA techniques within the skill of the art. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., James M. Cregg (Editor), Pichia Protocols (Methods in Molecular Biology), Humana Press (2010), Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999), Animal Cell Culture (R. I. Freshney, ed. (1986)); Immobilized Cells And Enzymes (IRL Press, (1986)); B. Perbal, A Practical Guide To Molecular Cloning (1984).

A “polynucleotide”, “nucleic acid” includes DNA and RNA in single stranded form, double-stranded form or otherwise.

A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein (e.g., promoters of the present invention) forms part of the present invention.

A “coding sequence” or a sequence “encoding” an expression product, such as an RNA or polypeptide is a nucleotide sequence (e.g., heterologous polynucleotide) that, when expressed, results in production of the product (e.g., a heterologous polypeptide such as an immunoglobulin heavy chain and/or light chain).

As used herein, the term “oligonucleotide” refers to a nucleic acid, generally of no more than about 100 nucleotides (e.g., 30, 40, 50, 60, 70, 80, or 90), that may be hybridizable to a polynucleotide molecule. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated.

A “protein”, “peptide” or “polypeptide” (e.g., a heterologous polypeptide such as an immunoglobulin heavy chain and/or light chain) includes a contiguous string of two or more amino acids.

A “protein sequence”, “peptide sequence” or “polypeptide sequence” or “amino acid sequence” refers to a series of two or more amino acids in a protein, peptide or polypeptide.

The term “isolated polynucleotide” or “isolated polypeptide” includes a polynucleotide or polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides set forth herein, e.g., the promoters set forth herein; and methods related thereto, e.g., as discussed herein.

An isolated polynucleotide or polypeptide will, preferably, be an essentially homogeneous composition of molecules but may contain some heterogeneity.

“Amplification” of DNA as used includes the use of polymerase chain reaction (PCR) to increase the concentration of a particular DNA sequence within a mixture of DNA sequences. For a description of PCR see Saiki, et al., Science (1988) 239:487.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence.

The present invention includes pmt2⁻, och1⁻ and pmt2⁻, och1⁻, pmt5⁻ Pichia cells comprising vectors or cassettes that comprise a heterologous polynucleotide which may be operably linked to a promoter. The term “vector” includes a vehicle (e.g., a plasmid) by which a DNA or RNA sequence can be introduced into a host cell, so as to transform the host and, optionally, promote expression and/or replication of the introduced sequence. Suitable vectors for use herein include plasmids, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

A polynucleotide (e.g., a heterologous polynucleotide, e.g., encoding an immunoglobulin heavy chain and/or light chain), operably linked to a promoter, may be expressed in an expression system. The term “expression system” means a host cell and compatible vector which, under suitable conditions, can express a protein or nucleic acid which is carried by the vector and introduced to the host cell. Expression systems include fungal or lower eukaryotic host cells (e.g., pmt2⁻, och1⁻ Pichia pastoris) and plasmid vectors, insect host cells and Baculovirus vectors, and mammalian host cells and vectors.

The term methanol-induction refers to increasing expression of a polynucleotide (e.g., a heterologous polynucleotide) operably linked to a methanol-inducible promoter in a host cell of the present invention by exposing the host cells to methanol. The present invention includes pmt2⁻, och1⁻ and pmt2⁻, och1, pmt5⁻ cells comprising a heterologous polynucleotide operably linked to a methanol-inducible promoter as well as methods of expressing a heterologous polypeptide encoded by the heterologous polynucleotide in the presence of methanol.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., J. Mol. Biol. (1991) 219:555-565; States, D. J., et al., Methods (1991) 3:66-70; Henikoff, S., et al., Proc. Natl. Acad. Sci. USA (1992)89:10915-10919; Altschul, S. F., et al., J. Mol. Evol. (1993) 36:290-300; ALIGNMENT STATISTICS: Karlin, S., et al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In an embodiment of the invention, Pichia pastoris PMT2 comprises the nucleotide sequence:

(SEQ ID NO: 1)
atgacaggccgtgtcgaccagaaatctgatcagaaggtgaaggaattgatcgaaaagatc
gactccgaatccacttccagagtttttcaggaagaaccagtcacttcgatcttgacacgt
tacgaaccctatgtcgccccaattatattcacgttgttgtcctttttcactcgtatgtac
aaaattgggatcaacaaccacgtcgtttgggatgaagctcacttcggaaagtttggctcc
tactatctcagacacgagttctaccacgatgtccaccctccgttgggtaagatgttggtc
ggtctatctggctacattgccggttacaatggctcctgggatttcccctccggtcaagag
taccctgactatattgattacgttaaaatgaggttattcaatgccaccttcagtgcctta
tgtgtgccattcgcctatttcaccatgaaggagattggatttgatatcaagacaacttgg
ctattcacactgatggtcttgtgtgaaacaagttattgtacgttaggaaaattcatcttg
ctggattcaatgctgctgctattcactgtgactacggttttcacctttgttaggttccat
aacgaaaacagtaaaccaggaaactcgttttctcgcaaatggtggaaatggcttctgctt
actggtatttccattggtctcacttgttccgtcaaaatggtgggtttatttgtcacagta
ttagttggaatttacacagttgttgacttatggaataaatttggtgatcaatccatttct
cgtaagaaatatgctgctcattggctagctcgtttcatcggcttgattgccatcccaatt
ggcgtttttctattgtcattccgtatccattttgaaatattatccaattctggtaccggt
gatgcaaacatgtcttcattgttccaagctaaccttcgtggatcatccgtcggaggaggc
cccagagatgtgaccactctcaactctaaagtgaccataaagagccaaggtttaggatct
ggtctgttacattcccacgttcaaacttatcctcaaggttccagccaacaacagattaca
acctattctcacaaagatgccaacaatgattgggtgtttcaacttacgagagaagactct
cgaaacgctttcaaggaagcccactatgtcgttgatggtatgtctgttcgtctcgttcat
tcaaacactggtagaaacttacacactcaccaagttgctgctcccgtctcctcatccgaa
tgggaagtcagttgttatggtaatgaaaccattggagacccgaaagataattggattgtt
gaaattgtcgaccagtatggtgatgaagataagctgagattgcacccattgacctccagt
ttccgtttgaaatcggcaactctgggatgctatttgggtacttcgggtgcttcactgcct
caatggggtttcagacaaggtgaagttgtttgttacaaaaatccgttccgtagagataag
cgcacctggtggaacatcgaggaccataacaatcctgatctacctaatcctccagaaaat
tttgttcttcccaggactcattttttgaaagactttgttcaattaaatttagcaatgatg
gcaacaaacaacgctttggtcccagacccagataaggaagataatctagcttcttctgcc
tgggaatggcccacgctacacgttggtatccgtctgtgcggttggggcgatgacaacgtc
aagtatttcttgattggttctcccgcaaccacctggacttcttcagttggtattgtagta
ttcctgttcctgctgttaatttacttgatcaaatggcaacgtcaatatgtcattttccca
tccgtccagactccactagagtcagccgacaccaaaacagttgcattgtttgacaagtct
gatagcttcaacgtcttccttatgggaggattatacccgcttctgggatggggtttacat
tttgctccgtttgtgatcatgtcgcgtgttacctacgttcaccattatcttcctgcattg
tactttgccatgattgttttctgctacttggtttctctgttggataagaaactaggccac
ccagcattaggattactgatctatgtggctctgtattccttggtcattggaacatttatt
tggctcagccccgttgtgtttggtatggacggtccgaacagaaattacagttacctaaac
cttctacctagttggagagtatcagaccca
In an embodiment of the invention, Pichia pastoris PMT2 polypeptide
comprises the amino acid sequence:
(SEQ ID NO: 2)
MTGRVDQKSDQKVKELIEKIDSESTSRVFQEEPVTSILTRYEPYVAPIIFTLLSFFTRMY
KIGINNHVVWDEAHFGKFGSYYLRHEFYHDVHPPLGKMLVGLSGYIAGYNGSWDFPSGQE
YPDYIDYVKMRLFNATFSALCVPFAYFTMKEIGFDIKTTWLFTLMVLCETSYCTLGKFIL
LDSMLLLFTVTTVFTFVRFHNENSKPGNSFSRKWWKWLLLTGISIGLTCSVKMVGLFVTV
LVGIYTVVDLWNKFGDQSISRKKYAAHWLARFIGLTATPIGVFLLSFRIHFEILSNSGTG
DANMSSLFQANLRGSSVGGGPRDVTTLNSKVTIKSQGLGSGLLHSHVQTYPQGSSQQQIT
TYSHKDANNDWVFQLTREDSRNAFKEAHYVVDGMSVRLVHSNTGRNLHTHQVAAPVSSSE
WEVSCYGNETIGDPKDNWIVEIVDQYGDEDKLRLHPLTSSFRLKSATLGCYLGTSGASLP
QWGFRQGEVVCYKNPFRRDKRTWWNIEDHNNPDLPNPPENFVLPRTHFLKDFVQLNLAMM
ATNNALVPDPDKEDNLASSAWEWPTLHVGIRLCGWGDDNVKYFLIGSPATTWTSSVGIVV
FLFLLLIYLIKWQRQYVIFPSVQTPLESADTKTVALFDKSDSFNVFLMGGLYPLLGWGLH
FAPFVIMSRVTYVHHYLPALYFAMIVFCYLVSLLDKKLGHPALGLLIYVALYSLVIGTFI
WLSPVVFGMDGPNRNYSYLNLLPSWRVSDP
In an embodiment of the invention, Pichia pastoris PMT5 comprises
the nucleotide sequence:
(SEQ ID NO: 17)
atgacattcttcttattagactgcctagttttgtataatcttacagaaattctagctcaagccctct
tacttgttcttcttctatgtcaactgattcctcaatatatgtggttggtggcccgcgaaatgactcc
tgagatatttggtcaaacctaccaaaggacaccacaccacagtactatagcacaacaatacatggcc
gcctttgagtacaaaaagggcattcaaagaccctatttttttaccaagccattggtgaaacctataa
cgctaagcggctttgaaaaaatacaattggctttgtttcttgcgttcacagtggccgtgagattctt
caatattcaataccccaaccaaattgtatttgatgaggtccattttggaaaatatgcccgaaactac
atcaatagctcatacttcatggatgtgcaccctcctttagtcaagatgctttacgccgccataggct
atttaggtggttacagaggagattttgttttcaacaagattggggataactacattggtaaagaggg
tgaaaaattggtaccctacgttttgatgcgatcgtttcccgcaatttgtggagtcttgattgttatt
ctttcttactttatccttagatacagcggatgccgacattttattgcactttttggagctttactgg
tttgtattgaaaactcattggtagctcaatcaagatttattctactagattctccattgcttttatt
cattgttctcacagtatacagttttgtgagattcagcaatgaaccagaaccttttggcaaaggctgg
ataagatatctatttttcactggtgtgtccttgggactcagtgtcagtagtaaatgggttggaatat
tcacaattggttggttaggagtcatgactgtaaaccaattgtggtggttaattggagacttaagcgt
tcccgatcgtgatgtggtaaagcatgtcttgtacagagcgtattttcttattatcctaccagtgatc
atttaccttggggtgtttgcaatccattttttggttctccatgaagctagtggcggttcaggtacag
tgagtcctagattcaaagccagtttggacggaactgatttttccaatctttatgctaacgtgtcttt
tggatccaccgtttcgataagacaccttggtacaggagagtttctacactcccacaaccacacatat
cctaaatcgcacaaccaacaggtaaccctatacggatacaaagactccaataatcttttcactattg
aaaagaaagataagctatctgacaaggaactattcggcgaggtatccttcctccgacacagagatgt
tataagattatttcacaagaaaacccaaggatatttgcacgtctctgattctagacctccaattagt
gagcaagagtacaacaatgaggtcagtattataggagacaaagactatgtccccgatgtcaatgaaa
actttgaggtgaagattatcaaagagtacagtgatgaagatgcaaagcatgaggttaaatccatcgg
aactgtgtttcaattattccataagggtaccaaatgtactctgtttggtcatcgtgtgaagctgcca
aaagactggggatttggtcaattggaggtcacttgtatcgagtcgccagtccttaaaaattctctgt
ggtacattgaagagaatacacacccacttttcaaccaaacatatcctgcaaaagtgaaagtcgaacc
cttaggattttttggcaagtttcttgagctgcaccaaaaaatgtggaaaacaaatgcaggcttgact
gcctctcacaagtatagctctagacccgaagattggcccgttcttgacagaggtgtgaactatttca
accgatcaggaaggacgatctacttgttaggtaacttgccaatctattggggaattgtatttactat
cggagtattcgttgttttcaagcttgttcagctctggaaatggaagccaaaccatgctccaacagta
accgatgcttcagctaaatatgattcccaatttttcatctactttgtcggttggctattccatttcg
ctccatcttttttgatggagcgacagctatttctgcaccactacataccatctctatggtttggtat
catatcaatcgctgtgctcagtgaatatgtttgggctaaactgggaaaaatcgtaggattcttctac
gttatgacaatattagggctttcgggtttcttcttctactggtatgccccaatcgtttatgggttag
agtggaacaaagacacctgtctgggttcgagactattaccaaactgggacatcccttgcgatcaatt
tcagtag
In an embodiment of the invention, Pichia pastoris PMT5 polypeptide
comprises the amino acid sequence
(SEQ ID NO: 18)
MTFFLLDCLVLYNLTEILAQALLLVLLLCQLIPQYMWLVAREMTPEIFGQTYQRTPHHSTIAQQYMA
AFEYKKGIQRPYFFTKPLVKPITLSGFEKIQLALFLAFTVAVRFFNIQYPNQIVFDEVHFGKYARNY
INSSYFMDVHPPLVKMLYAAIGYLGGYRGDFVFNKIGDNYIGKEGEKLVPYVLMRSFPAICGVLIVI
LSYFILRYSGCRHFIALFGALLVCIENSLVAQSRFILLDSPLLLFIVLTVYSFVRFSNEPEPFGKGW
IRYLFFTGVSLGLSVSSKWVGIFTIGWLGVMTVNQLWWLIGDLSVPDRDVVKHVLYRAYFLIILPVI
IYLGVFAIHFLVLHEASGGSGTVSPRFKASLDGTDFSNLYANVSFGSTVSIRHLGTGEFLHSHNHTY
PKSHNQQVTLYGYKDSNNLFTIEKKDKLSDKELFGEVSFLRHRDVIRLFHKKTQGYLHVSDSRPPIS
EQEYNNEVSIIGDKDYVPDVNENFEVKIIKEYSDEDAKHEVKSIGTVFQLFHKGTKCTLFGHRVKLP
KDWGFGQLEVTCIESPVLKNSLWYIEENTHPLFNQTYPAKVKVEPLGFFGKFLELHQKMWKTNAGLT
ASHKYSSRPEDWPVLDRGVNYFNRSGRTIYLLGNLPIYWGIVFTIGVFVVFKLVQLWKWKPNHAPTV
TDASAKYDSQFFIYFVGWLFHFAPSFLMERQLFLHHYIPSLWFGIISIAVLSEYVWAKLGKIVGFFY
VMTILGLSGFFFYWYAPIVYGLEWNKDTCLGSRLLPNWDIPCDQFQ
In an embodiment of the invention, Pichia pastoris OCH1 comprises
the nucleotide sequence:
(SEQ ID NO: 3)
atggctatattcgccgtttctgtcatttgcgttttgtacggaccctcacaacaattatca
tctccaaaaatagactatgatccattgacgctccgatcacttgatttgaagactttggaa
gctccttcacagttgagtccaggcaccgtagaagataatcttcgaagacaattggagttt
cattttccttaccgcagttacgaaccttttccccaacatatttggcaaacgtggaaagtt
tctccctctgatagttcctttccgaaaaacttcaaagacttaggtgaaagttggctgcaa
aggtccccaaattatgatcattttgtgatacccgatgatgcagcatgggaacttattcac
catgaatacgaacgtgtaccagaagtcttggaagctttccacctgctaccagagcccatt
ctaaaggccgattttttcaggtatttgattctttttgcccgtggaggactgtatgctgac
atggacactatgttattaaaaccaatagaatcgtggctgactttcaatgaaactattggt
ggagtaaaaaacaatgctgggttggtcattggtattgaggctgatcctgatagacctgat
tggcacgactggtatgctagaaggatacaattttgccaatgggcaattcagtccaaacga
ggacacccagcactgcgtgaactgattgtaagagttgtcagcacgactttacggaaagag
aaaagcggttacttgaacatggtggaaggaaaggatcgtggaagtgatgtgatggactgg
acgggtccaggaatatttacagacactctatttgattatatgactaatgtcaatacaaca
ggccactcaggccaaggaattggagctggctcagcgtattacaatgccttatcgttggaa
gaacgtgatgccctctctgcccgcccgaacggagagatgttaaaagagaaagtcccaggt
aaatatgcacagcaggttgttttatgggaacaatttaccaacctgcgctcccccaaatta
atcgacgatattcttattcttccgatcaccagcttcagtccagggattggccacagtgga
gctggagatttgaaccatcaccttgcatatattaggcatacatttgaaggaagttggaag
gac
In an embodiment of the invention, Pichia pastoris OCH1 comprises
the amino acid sequence:
(SEQ ID NO: 4)
MAIFAVSVICVLYGPSQQLSSPKIDYDPLTLRSLDLKTLEAPSQLSPGTVEDNLRRQLEF
HFPYRSYEPFPQHIWQTWKVSPSDSSFPKNEKDLGESWLQRSPNYDHEVIPDDAAWELIH
HEYERVPEVLEAFHLLPEPILKADFFRYLILFARGGLYADMDTMLLKPIESWLTENETIG
GVKNNAGLVIGIEADPDRPDWHDWYARRIQFCQWAIQSKRGHPALRELIVRVVSTTLRKE
KSGYLNMVEGKDRGSDVMDWTGPGIFTDTLFDYMTNVNTTGHSGQGIGAGSAYYNALSLE
ERDALSARPNGEMLKEKVPGKYAQQVVLWEQFTNLRSPKLIDDILILPITSFSPGIGHSG
AGDLNHHLAYIRHTFEGSWKD

The identities of PMT2, PMT5 and OCH1 are known in the art. Specific examples, of PMT2, PMT5 and OCH1 are set forth herein (SEQ ID NOs: 1-4, 17, 18). In an embodiment of the invention, Pichia pastoris PMT2, PMT5 and/or OCH1 polypeptide comprises at least about 90% (e.g., 90%, 91%, 92%, 93%, 94%, 95%, 96%, 97%, 98%, or 99%) sequence similarity or identity to SEQ ID NO: 2, 4 or 18, respectively. In an embodiment of the invention, Pichia pastoris PMT2, PMT5 and/or OCH1 polynucleotide comprises at least about 90% (e.g., 90%, 91%, 92%, 93%, 94%, 95%, 96%, 97%, 98%, or 99%) sequence identity to SEQ ID NO: 1, 3 or 17, respectively.

### Host Cells

The present invention encompasses any isolated fungal or lower eukaryotic host cells, e.g., Pichia host cell (e.g., such as Pichia pastoris), comprising a pmt2⁻, och1⁻ double mutation or pmt2⁻, och1⁻, pmt5⁻ triple mutation, including host cells comprising a promoter e.g., operably linked to a polynucleotide encoding a heterologous polypeptide (e.g., a reporter or immunoglobulin heavy and/or light chain) as well as methods of use thereof, e.g., methods for expressing the heterologous polypeptide in the host cell. Host cells of the present invention, may be also genetically engineered so as to express particular glycosylation patterns on polypeptides that are expressed in such cells. Host cells of the present invention are discussed in detail herein.

In an embodiment of the invention, an isolated fungal or lower eukaryotic host cells, e.g., Pichia cell, that lacks functional PMT2 polypeptide and also lacks functional OCH1 polypeptide, and, optionally lacks functional PMT5 polypeptide, that includes a heterologous polynucleotide encoding a heterologous polypeptide that is an immunoglobulin (e.g., light and heavy chain immunoglobulins, for example, that are in an anti-HER2 antibody, e.g., operably linked to a promoter), secretes at least 2-fold more properly folded tetrameric recombinant heterologous immunoglobulin polypeptide and/or produces more homogenous low O-glycan heterologous immunoglobulin polypeptide (e.g., as evaluated by SDS-PAGE analysis), than that of an isolated Pichia cell that comprises functional PMT2 and OCH1 and, optionally, PMT5 polypeptide (e.g., as evaluated by HPLC analysis of the cell culture supernatant). In an embodiment of the invention, for O-glycosylation, an och1⁻, pmt2⁻ double mutant or och1⁻, pmt2⁻, pmt5⁻ triple mutant produces antibody with fewer than 2, 3, 4 or 5 ser/thr residues O-glycosylated per mAb (H2/L2) when in the absence of chemical PMT inhibitor.

In an embodiment of the invention, a pmt2⁻ knock-out lower eukaryotic or fungal host cell (e.g., pmt2⁻, och1⁻ or pmt2⁻, och1⁻, pmt5⁻) exhibits resistance to a Pmt inhibitor. Such inhibitors are typically used to reduce the amount of O-glycosylation of recombinant heterologous proteins produced by host cells but also have the effect of reducing the robustness of the host cells during fermentation. In an embodiment of the invention, the level of O-glycosylation of a heterologous protein expressed in a pmt2⁻ (e.g., pmt2⁻, och1 or pmt2⁻, och1, pmt5⁻) host cell in the presence or absence of a PMT inhibitor is about equal (e.g., a difference of within about 10%, 25%, 75%, 50%, 100% or 150%).

In an embodiment of the invention, PMT2 knock-out host cells (e.g., pmt2⁻, och1⁻ or pmt2⁻, och1⁻, pmt5⁻) express PMT2 having a mutation in the PMT2 conserved region Pro Phe Val Ile Met Ser Arg Val Thr Tyr Val His His Tyr Leu Pro Ala Leu Tyr Phe Ala (amino acids 663-683 of SEQ ID NO: 2), e.g., wherein a serine residue replaces the phenylalanine residue at position 2 of the conserved PMT2 region: Pro Phe Val Ile Met Ser Arg Val Thr Tyr Val His His Tyr Leu Pro Ala Leu Tyr Phe Ala (amino acids 663-683 of SEQ ID NO: 2).

In an embodiment of the invention, the endogenous PMT2 gene in a pmt2⁻ fungal or lower eukaryotic host cell (e.g., pmt2⁻, och1⁻ or pmt2⁻, och1⁻, pmt5⁻) has a single point-mutation wherein a “T” to a “C” nucleotide transition occurs at position 1991 in the open reading frame (ORF) encoding the Pmt2 protein (PMT2-T1991C point mutation), which results in an amino acid change at position 664 of the Pmt2p from phenylalanine encoded by the codon TTT to serine encoded by the codon TCT (Pmt2p-F664S mutant protein). If the fungal or lower eukaryotic host cell is a pmt2⁻ Saccharomyces cerevisiae, in an embodiment of the invention, the PMT2 gene has a F666S mutation (Pmt2p-F666S mutant protein).

The term “eukaryotic” refers to a nucleated cell or organism, and includes insect cells, plant cells, mammalian cells, animal cells and lower eukaryotic cells.

The term “lower eukaryotic cells” includes fungal cells (e.g., pmt2⁻, och1⁻ or pmt2⁻, och1⁻, pmt5), which include yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa.

Isolated fungal host cells of the present invention (e.g., pmt2⁻, och1⁻ or pmt2⁻, och1⁻, pmt5) are cells belonging to the Fungi kingdom. In an embodiment of the invention, the fungal host cell is selected from the group consisting of any Pichia cell, such as Pichia pastoris, Pichia angusta (Hansenula polymorpha), Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis or Pichia methanolica; Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

The scope of the present invention encompasses an isolated pmt2⁻, och1⁻ or pmt2⁻, och1⁻, pmt5⁻ Pichia cell that has been produced by any method. In an embodiment of the invention, however, the cell is generated using a method such as the following: expressing a site-specific recombinase in an och1⁻ PMT2 or och1⁻, pmt5, PMT2 Pichia cell wherein the endogenous, chromosomal PMT2 locus (e.g., the PMT2 gene coding sequence (open reading frame) and/or regulatory sequences such as the promoter; or any portion thereof; optionally including neighboring 5′ and/or 3′ sequences on the chromosomal) is flanked by target sites recognized by the recombinase such that recombination of the sites deletes PMT2, e.g., wherein the method comprises expression of Cre that is operably linked to an inducible promoter, such as the AOX1 promoter, wherein expression of the inducible promoter is induced, e.g., if the promoter is the AOX1 promoter, then induction is in the presence of methanol; and wherein LoxP sites (e.g., ATAACTTCGTATA-GCATACAT-TATACGAAGTTAT) are at the 5′ and 3′ side of the endogenous chromosomal PMT2 in the cell; and wherein the Cre recombinase, when expressed in the cell, recombines the LoxP sites such that the PMT2 is deleted from the chromosome. This method for generating a pmt2⁻, och1⁻ or pmt2⁻, och1⁻, pmt5⁻Pichia cell is part of the present invention along with host cells that are the product of such a process. Kuhn & Torres, Methods Mol. Biol. 180: 175-204 (2002).

In another embodiment of the invention, the cell is generated using the following method: mutating endogenous PMT2 in an och1⁻ or och1⁻, pmt5⁻Pichia cell that comprises PMT2 operably linked to an inducible promoter (e.g., AOX1) under conditions whereby the promoter is induced (e.g., in the presence of methanol if the promoter is AOX1) and then, after the endogenous, chromosomal PMT2 is mutated, culturing the cell under conditions whereby the promoter is not induced. This method for generating a pmt2⁻, och1⁻ or pmt2⁻, och1⁻, pmt5⁻Pichia cell is also part of the present invention along with host cells that are the product of such a process.

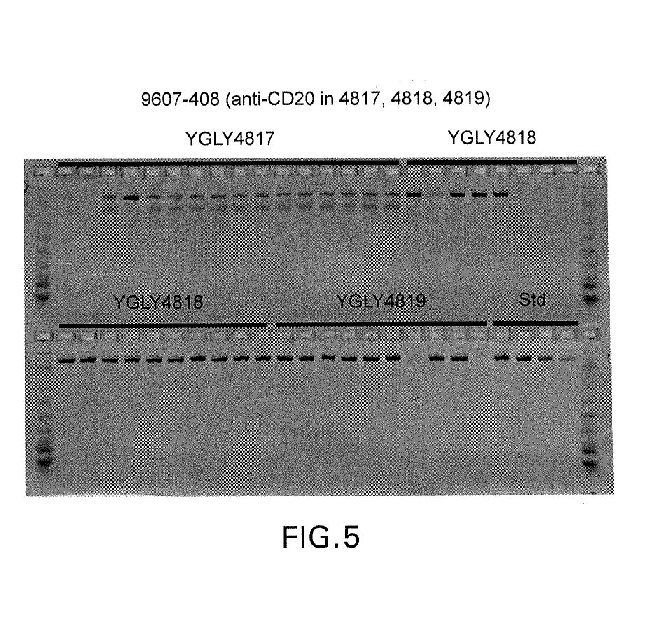

OCH1 can be mutated using methods that are known in the art, see, example International Patent Application Publication No. WO2011/106389. For example, in an embodiment of the invention, plasmid pGLY40 (FIG. 5 of WO2011/106389) is used for this purpose. pGLY40 is an integration vector that targets the OCH1 locus and contains a nucleic acid molecule comprising the P. pastoris URA5 gene or transcription unit, e.g.,

(SEQ ID NO: 13)
tctagaggga cttatctggg tccagacgat gtgtatcaaa agacaaatta gagtatttat
aaagttatgt aagcaaatag gggctaatag ggaaagaaaa attttggttc tttatcagag
ctggctcgcg cgcagtgttt ttcgtgctcc tttgtaatag tcatttttga ctactgttca
gattgaaatc acattgaaga tgtcactgga ggggtaccaa aaaaggtttt tggatgctgc
agtggcttcg caggccttga agtttggaac tttcaccttg aaaagtggaa gacagtctcc
atacttcttt aacatgggtc ttttcaacaa agctccatta gtgagtcagc tggctgaatc
ttatgctcag gccatcatta acagcaacct ggagatagac gttgtatttg gaccagctta
taaaggtatt cctttggctg ctattaccgt gttgaagttg tacgagctgg gcggcaaaaa
atacgaaaat gtcggatatg cgttcaatag aaaagaaaag aaagaccacg gagaaggtgg
aagcatcgtt ggagaaagtc taaagaataa aagagtactg attatcgatg atgtgatgac
tgcaggtact gctatcaacg aagcatttgc tataattgga gctgaaggtg ggagagttga
aggttgtatt attgccctag atagaatgga gactacagga gatgactcaa ataccagtgc
tacccaggct gttagtcaga gatatggtac ccctgtcttg agtatagtga cattggacca
tattgtggcc catttgggcg aaactttcac agcagacgag aaatctcaaa tggaaacgta
tagaaaaaag tatttgccca aataagtatg aatctgcttc gaatgaatga attaatccaa
ttatcttctc accattattt tcttctgttt cggagctttg ggcacggcgg cggatcc;
flanked by nucleic acid molecules comprising lacZ repeats, e.g.,
(SEQ ID NO: 14)
cctgcactgg atggtggcgc tggatggtaa gccgctggca agcggtgaag tgcctctgga
tgtcgctcca caaggtaaac agttgattga actgcctgaa ctaccgcagc cggagagcgc
cgggcaactc tggctcacag tacgcgtagt gcaaccgaac gcgaccgcat ggtcagaagc
cgggcacatc agcgcctggc agcagtggcg tctggcggaa aacctcagtg tgacgctccc
cgccgcgtcc cacgccatcc cgcatctgac caccagcgaa atggattttt gcatcgagct
gggtaataag cgttggcaat ttaaccgcca gtcaggcttt ctttcacaga tgtggattgg
cgataaaaaa caactgctga cgccgctgcg cgatcagttc acccgtgcac cgctggataa
cgacattggc gtaagtgaag cgacccgcat tgaccctaac gcctgggtcg aacgctggaa
ggcggcgggc cattaccagg ccgaagcagc gttgttgcag tgcacggcag atacacttgc
tgatgcggtg ctgattacga ccgctcacgc gtggcagcat caggggaaaa ccttatttat
cagccggaaa acctaccgga ttgatggtag tggtcaaatg gcgattaccg ttgatgttga
agtggcgagc gatacaccgc atccggcgcg gattggcctg aactgccag;
which in turn is flanked on one side by a nucleic acid
molecule comprising a nucleotide sequence from the 5′ region of
the OCH1 gene, e.g.,
(SEQ ID NO: 15)
aaaacctttt ttcctattca aacacaaggc attgcttcaa cacgtgtgcg tatccttaac
acagatactc catacttcta ataatgtgat agacgaatac aaagatgttc actctgtgtt
gtgtctacaa gcatttctta ttctgattgg ggatattcta gttacagcac taaacaactg
gcgatacaaa cttaaattaa ataatccgaa tctagaaaat gaacttttgg atggtccgcc
tgttggttgg ataaatcaat accgattaaa tggattctat tccaatgaga gagtaatcca
agacactctg atgtcaataa tcatttgctt gcaacaacaa acccgtcatc taatcaaagg
gtttgatgag gcttaccttc aattgcagat aaactcattg ctgtccactg ctgtattatg
tgagaatatg ggtgatgaat ctggtcttct ccactcagct aacatggctg tttgggcaaa
ggtggtacaa ttatacggag atcaggcaat agtgaaattg ttgaatatgg ctactggacg
atgcttcaag gatgtacgtc tagtaggagc cgtgggaaga ttgctggcag aaccagttgg
cacgtcgcaa caatccccaa gaaatgaaat aagtgaaaac gtaacgtcaa agacagcaat
ggagtcaata ttgataacac cactggcaga gcggttcgta cgtcgttttg gagccgatat
gaggctcagc gtgctaacag cacgattgac aagaagactc tcgagtgaca gtaggttgag
taaagtattc gcttagattc ccaaccttcg ttttattctt tcgtagacaa agaagctgca
tgcgaacata gggacaactt ttataaatcc aattgtcaaa ccaacgtaaa accctctggc
accattttca acatatattt gtgaagcagt acgcaatatc gataaatact caccgttgtt
tgtaacagcc ccaacttgca tacgccttct aatgacctca aatggataag ccgcagcttg
tgctaacata ccagcagcac cgcccgcggt cagctgcgcc cacacatata aaggcaatct
acgatcatgg gaggaattag ttttgaccgt caggtcttca agagttttga actcttcttc
ttgaactgtg taacctttta aatgacggga tctaaatacg tcatggatga gatcatgtgt
gtaaaaactg actccagcat atggaatcat tccaaagatt gtaggagcga acccacgata
aaagtttccc aaccttgcca aagtgtctaa tgctgtgact tgaaatctgg gttcctcgtt
gaagaccctg cgtactatgc ccaaaaactt tcctccacga gccctattaa cttctctatg
agtttcaaat gccaaacgga cacggattag gtccaatggg taagtgaaaa acacagagca
aaccccagct aatgagccgg ccagtaaccg tcttggagct gtttcataag agtcattagg
gatcaataac gttctaatct gttcataaca tacaaatttt atggctgcat agggaaaaat
tctcaacagg gtagccgaat gaccctgata tagacctgcg acaccatcat acccatagat
ctgcctgaca gccttaaaga gcccgctaaa agacccggaa aaccgagaga actctggatt
agcagtctga aaaagaatct tcactctgtc tagtggagca attaatgtct tagcggcact
tcctgctact ccgccagcta ctcctgaata gatcacatac tgcaaagact gcttgtcgat
gaccttgggg ttatttagct tcaagggcaa tttttgggac attttggaca caggagactc
agaaacagac acagagcgtt ctgagtcctg gtgctcctga cgtaggccta gaacaggaat
tattggcttt atttgtttgt ccatttcata ggcttggggt aatagataga tgacagagaa
atagagaaga cctaatattt tttgttcatg gcaaatcgcg ggttcgcggt cgggtcacac
acggagaagt aatgagaaga gctggtaatc tggggtaaaa gggttcaaaa gaaggtcgcc
tggtagggat gcaatacaag gttgtcttgg agtttacatt gaccagatga tttggctttt
tctctgttca attcacattt ttcagcgaga atcggattga cggagaaatg gcggggtgtg
gggtggatag atggcagaaa tgctcgcaat caccgcgaaa gaaagacttt atggaataga
actactgggt ggtgtaagga ttacatagct agtccaatgg agtccgttgg aaaggtaaga
agaagctaaa accggctaag taactaggga agaatgatca gactttgatt tgatgaggtc
tgaaaatact ctgctgcttt ttcagttgct ttttccctgc aacctatcat tttccttttc
ataagcctgc cttttctgtt ttcacttata tgagttccgc cgagacttcc ccaaattctc
tcctggaaca ttctctatcg ctctccttcc aagttgcgcc ccctggcact gcctagtaat
attaccacgc gacttatatt cagttccaca atttccagtg ttcgtagcaa atatcatcag
ccatggcgaa ggcagatggc agtttgctct actataatcc tcacaatcca cccagaaggt
attacttcta catggctata ttcgccgttt ctgtcatttg cgttttgtac ggaccctcac
aacaattatc atctccaaaa atagactatg atccattgac gctccgatca cttgatttga
agactttgga agctccttca cagttgagtc caggcaccgt agaagataat cttcg;
and on the other side by a nucleic acid molecule comprising a
nucleotide sequence from the 3′ region of the OCH1 gene, e.g.,
(SEQ ID NO: 16)
aaagctagag taaaatagat atagcgagat tagagaatga ataccttctt ctaagcgatc
gtccgtcatc atagaatatc atggactgta tagttttttt tttgtacata taatgattaa
acggtcatcc aacatctcgt tgacagatct ctcagtacgc gaaatccctg actatcaaag
caagaaccga tgaagaaaaa aacaacagta acccaaacac cacaacaaac actttatctt
ctccccccca acaccaatca tcaaagagat gtcggaacca aacaccaaga agcaaaaact
aaccccatat aaaaacatcc tggtagataa tgctggtaac ccgctctcct tccatattct
gggctacttc acgaagtctg accggtctca gttgatcaac atgatcctcg aaatgggtgg
caagatcgtt ccagacctgc ctcctctggt agatggagtg ttgtttttga caggggatta
caagtctatt gatgaagata ccctaaagca actgggggac gttccaatat acagagactc
cttcatctac cagtgttttg tgcacaagac atctcttccc attgacactt tccgaattga
caagaacgtc gacttggctc aagatttgat caatagggcc cttcaagagt ctgtggatca
tgtcacttct gccagcacag ctgcagctgc tgctgttgtt gtcgctacca acggcctgtc
ttctaaacca gacgctcgta ctagcaaaat acagttcact cccgaagaag atcgttttat
tcttgacttt gttaggagaa atcctaaacg aagaaacaca catcaactgt acactgagct
cgctcagcac atgaaaaacc atacgaatca ttctatccgc cacagatttc gtcgtaatct
ttccgctcaa cttgattggg tttatgatat cgatccattg accaaccaac ctcgaaaaga
tgaaaacggg aactacatca aggtacaagg ccttcca.

In this embodiment, according to WO2011/106389, plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

In an embodiment of the invention, an isolated pmt2⁻, och1⁻ or pmt2⁻, och1⁻, pmt5⁻ fungal or lower eukaryotic host cell, such as a Pichia cell (e.g., Pichia pastoris), is genetically engineered to include a nucleic acid that encodes an α-1,2-mannosidase that has a signal peptide that directs it for secretion. For example, in an embodiment of the invention, the pmt2⁻, och1⁻ or pmt2⁻, och1⁻, pmt5⁻ host cell is engineered to express an exogenous α-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield Man₅GlcNAc₂. See U.S. Pat. No. 7,029,872. The present invention includes methods for producing one or more heterologous polypeptides comprising (i) introducing a polynucleotide encoding the heterologous polypeptide(s) into such a pmt2⁻, och1⁻, α-1,2-mannosidase⁺ (optionally pmt5⁻) host cell and (ii) culturing the host cell under conditions favorable to expression of the heterologous polypeptide(s) in the cell and, optionally, (iii) isolating the heterologous polypeptide(s) from the host cell. The invention also encompasses a method for producing a heterologous recombinant glycoprotein comprising an N-glycan structure that comprises a Man₅GlcNAc₂ glycoform in a pmt2⁻, och1⁻ or pmt2⁻, och1⁻, pmt5⁻ fungal or lower eukaryotic host cell that does not display alpha-1,6 mannosyltransferase activity with respect to the N-glycan on a glycoprotein, the method comprising the step of introducing into the pmt2⁻, och1⁻ or pmt2⁻, och1⁻, pmt5⁻ fungal or lower eukaryotic host cell, a polynucleotide encoding the heterologous recombinant glycoprotein, and a polynucleotide encoding an alpha-1,2 mannosidase enzyme selected to have optimal activity in the ER or Golgi of said host cell, the enzyme comprising: (a) an alpha-1,2 mannosidase catalytic domain having optimal activity in said ER or Golgi at a pH between 5.1 and 8.0; fused to (b) a cellular targeting signal peptide not normally associated with the catalytic domain selected to target the mannosidase enzyme to the ER or Golgi apparatus of the host cell; and culturing the fungal or lower eukaryotic host cell under conditions favorable to expression of the heterologous recombinant glycoprotein, whereby, upon expression and passage of the heterologous recombinant glycoprotein through the ER or Golgi apparatus of the host cell, in excess of 30 mole % of the N-glycan structures attached thereto have a Man₅GlcNAc₂ glycoform that can serve as a substrate for GlcNAc transferase I in vivo.

Isolated pmt2⁻, och1⁻ or pmt2⁻, och1⁻, pmt5⁻ fungal or lower eukaryotic host cells of the present invention, such as Pichia host cells (e.g., Pichia pastoris) are, in an embodiment of the invention, genetically engineered to eliminate glycoproteins having alpha-mannosidase-resistant N-glycans by mutating one or more of the β-mannosyltransferase genes (e.g., BMTI, BMT2, BMT3, and/or BMT4) (See, U.S. Pat. No. 7,465,577) or abrogating translation of RNAs encoding one or more of the beta-mannosyltransferases using interfering RNA, antisense RNA, or the like. The scope of the present invention includes methods for producing one or more heterologous polypeptides comprising (i) introducing a polynucleotide encoding the heterologous polypeptide(s) into such a pmt2⁻, och1⁻, β-mannosyltransferase⁻ (optionally pmt5) (e.g., bmt1⁻, bmt2⁻, bmt3⁻, and/or bmt4⁻) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells (e.g., Pichia, e.g., Pichia pastoris) of the present invention also include those that are genetically engineered to eliminate glycoproteins having phosphomannose residues, e.g., by deleting or disrupting one or both of the phosphomannosyl transferase genes PNO1 and MNN4B (See for example, U.S. Pat. Nos. 7,198,921 and 7,259,007), which can include deleting or disrupting one or more of the phosphomannosyltransferases or abrogating translation of RNAs encoding one or more of the phosphomannosyltransferases using interfering RNA, antisense RNA, or the like. In an embodiment of the invention, such fungal or lower eukaryotic host cells produce glycoproteins that have predominantly an N-glycan selected from the group consisting of complex N-glycans, hybrid N-glycans, and high mannose N-glycans wherein complex N-glycans are, in an embodiment of the invention, selected from the group consisting of Man₃GlcNAc₂, GlcNAC_{(I-4)}Man₃GlcNAc₂, NANA_{(I-4)}GlcNAc_{(I-4)}Man₃GlcNAc₂, and NANA_{(I-4)}Gal₍₁₋₄₎Man₃GlcNAc₂; hybrid N-glycans are, in an embodiment of the invention, selected from the group consisting of Man₅GlcNAc₂, GlcNAcMan₅GlcNAc₂, GalGlcNAcMan₅GlcNAc₂, and NANAGalGlcNAcMan₅GlcNAc₂; and high mannose N-glycans are, in an embodiment of the invention, selected from the group consisting of Man₆GlcNAc₂, Man₇GlcNAc₂, Man₈GlcNAc₂, and Man₉GlcNAc₂. The scope of the present invention includes methods for producing one or more heterologous polypeptides comprising (i) introducing a polynucleotide encoding the heterologous polypeptide(s) into such a pmt2⁻, och1⁻, phosphomannosyl transferase⁻ (e.g., pno1⁻ and/or mnn4b⁻) (optionally pmt5⁻) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells, such as Pichia host cells (e.g., Pichia pastoris) of the present invention include those that are genetically engineered to include a nucleic acid that encodes the Leishmania sp. single-subunit oligosaccharyltransferase STT3A protein, STT3B protein, STT3C protein, STT3D protein, or combinations thereof such as those described in WO2011/06389. The scope of the present invention includes methods for producing one or more heterologous polypeptides comprising (i) introducing a polynucleotide encoding the heterologous polypeptide(s) into such a pmt2⁻, och1⁻, (Leishmania STT3A⁺, Leishmania STT3B⁺, Leishmania STT3C⁺, and/or Leishmania STT3D⁺) (optionally pmt5) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells (e.g., Pichia pastoris) of the present invention also include those that are genetically engineered to eliminate nucleic acids encoding dolichol-P-Man dependent alpha(1-3) mannosyltransferase, e.g., Alg3, such as described in U.S. Patent Publication No. US2005/0170452. The scope of the present invention includes methods for producing one or more heterologous polypeptides comprising (i) introducing a polynucleotide encoding the heterologous polypeptide(s) into such a pmt2⁻, och1⁻, Alg3⁻ (optionally pmt5) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells of the present invention, such as Pichia cells (e.g., Pichia pastoris) expressing a polypeptide having an endomannosidase activity (e.g., human (e.g., human liver), rat or mouse endomanosidase) that is targeted to a vesicular compartment within the host cell are part of the present invention. The scope of the present invention includes methods for producing one or more heterologous polypeptides comprising (i) introducing a polynucleotide encoding the heterologous polypeptide(s) into such a pmt2⁻, och1⁻, endomannosidase⁺ (optionally pmt5) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells, such as Pichia cells (e.g., Pichia pastoris) of the present invention are, in an embodiment of the invention, engineered for producing a recombinant sialylated glycoprotein in the host cell, e.g., wherein the host cell is selected or engineered to produce recombinant glycoproteins comprising a glycoform selected from the group consisting of Gal₍₁₋₄₎GlcNAc₍₁₋₄₎Man₃GlcNAc₂, e.g., by a method comprising: (a) transforming, into the pmt2⁻, och1⁻ or pmt2⁻, och1⁻, pmt5⁻ fungal or lower eukaryotic host cell, one or more polynucleotides encoding a bifunctional UDP-N-acetylglucosamine-2-epimerase/N-acetylmannosamine kinase, an N-acetylneuraminate-9-phosphate synthase, and a CMP-sialic acid synthase; (b) transforming into the host cell a polynucleotide encoding a CMP-sialic acid transporter; and (c) transforming into the host cell a polynucleotide molecule encoding a 2,6-sialyltransferase catalytic domain fused to a cellular targeting signal peptide, e.g., encoded by nucleotides 1-108 of the S. cerevisiae Mnn2; wherein, upon passage of a recombinant glycoprotein through the secretory pathway of the host cell, a recombinant sialylated glycoprotein comprising a glycoform selected from the group consisting of NANA₍₁₋₄₎Gal₍₁₋₄₎GlcNAc₍₁₋₄₎Man₃GlcNAc₂ glycoform is produced. The scope of the present invention includes methods for producing one or more heterologous polypeptides comprising (i) introducing a polynucleotide encoding the heterologous polypeptide(s) into such a pmt2⁻, och1⁻, bifunctional UDP-N-acetylglucosamine-2-epimerase/N-acetylmannosamine kinase⁺, N-acetylneuraminate-9-phosphate synthase⁺, CMP-Sialic acid synthase⁺, CMP-sialic acid transporter⁺, 2,6-sialyltransferase⁺ (optionally pmt5) fungal or lower eukaryotic host cell and (ii) culturing the host cell under conditions favorable to expression of the heterologous polypeptide(s) in the cell and, optionally, (iii) isolating the heterologous polypeptide(s) from the host cell.

In addition, isolated pmt2⁻, och1⁻ or pmt2⁻, och1⁻, pmt5⁻ fungal or lower eukaryotic host cells of the present invention, such as Pichia cells (e.g., Pichia pastoris), are, in an embodiment of the invention, engineered for generating galactosylated proteins, e.g., having a terminal galactose residue and essentially lacking fucose and sialic acid residues on the glycoprotein. In one embodiment of the present invention, the isolated pmt2⁻, och1⁻ or pmt2⁻, och1⁻, pmt5⁻ fungal or lower eukaryotic host cell comprises an isolated nucleic acid molecule encoding β-galactosyltransferase activity and at least a polynucleotide encoding UDP-galactose transport activity, UDP-galactose C4 epimerase activity, galactokinase activity or galactose-1-phosphate uridyl transferase, e.g., wherein the host cell is genetically engineered to produce N-linked oligosaccharides having terminal GlcNAc residues and comprising a polynucleotide encoding a fusion protein that in the host cell transfers a galactose residue from UDP-galactose onto a terminal GlcNAc residue of an N-linked oligosaccharide branch of an N-glycan of a glycoprotein, wherein the N-linked oligosaccharide branch is selected from the group consisting of GlcNAcβ1,2-Manα1; GlcNAcβ1,4-Manα1,3, GlcNAcβ1,2-Manα1,6, GlcNAcβ1,4-Manα1,6 and GlcNAcβ1,6-Manα1,6; wherein the host cell is diminished or depleted in dolichyl-P-Man:Man₅GlcNAc₂-PP-dolichyl α-1,3 mannosyltransferase activity, and wherein the host cell produces a glycoprotein having one or more galactose residues. The scope of the present invention includes methods for producing one or more heterologous polypeptides comprising (i) introducing a polynucleotide encoding the heterologous polypeptide(s) into such a host cell that is engineered for generating galactosylated proteins and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells of the present invention, such as Pichia cells (e.g., Pichia pastoris) expressing a galactosyltransferase e.g., an alpha 1,3-galactosyltransferase or a beta 1,4-galactosyltransferase are part of the present invention. The scope of the present invention includes methods for producing one or more heterologous polypeptides comprising (i) introducing a polynucleotide encoding the heterologous polypeptide(s) into such a pmt2⁻, och1⁻, galactosyltransferase⁺ (optionally pmt5⁻) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells of the present invention, such as Pichia cells (e.g., Pichia pastoris) expressing a nucleotide sugar transporter are part of the present invention. The scope of the present invention includes methods for producing one or more heterologous polypeptides comprising (i) introducing a polynucleotide encoding the heterologous polypeptide(s) into such a pmt2⁻, och1⁻, nucleotide sugar transporter⁺ (optionally pmt5⁻) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells of the present invention, such as Pichia cells (e.g., Pichia pastoris) expressing a sialyltransferase are part of the present invention. The scope of the present invention includes methods for producing one or more heterologous polypeptides comprising (i) introducing a polynucleotide encoding the heterologous polypeptide(s) into such a pmt2⁻, och1⁻, sialyltransferase⁺ (optionally pmt5⁻) host cell and (ii) culturing the host cell under conditions favorable to expression of the heterologous polypeptide(s) in the cell and, optionally, (iii) isolating the heterologous polypeptide(s) from the host cell.

Isolated pmt2⁻, och1⁻ or pmt2⁻, och1⁻, pmt5⁻ fungal or lower eukaryotic host cells of the present invention, such as Pichia cells (e.g., Pichia pastoris) expressing an acetylglucosaminyl transferase, e.g., GNT1 or GNT2 or GNT4 are part of the present invention. The scope of the present invention includes methods for producing one or more heterologous polypeptides comprising (i) introducing a polynucleotide encoding the heterologous polypeptide(s) into such a pmt2⁻, och1⁻, acetylglucosaminyl transferase⁺ (optionally pmt5⁻) host cell and (ii) culturing the host cell under conditions favorable to expression of the heterologous polypeptide(s) in the cell and, optionally, (iii) isolating the heterologous polypeptide(s) from the host cell.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” “PNGase”, or “glycanase” or “glucosidase” refer to peptide N-glycosidase F (EC 3.2.2.18).

In an embodiment of the invention, a fungal or lower eukaryotic host cell is pmt2⁻, och1⁻ (optionally pmt5⁻) and (1) bmt1⁻, bmt2⁻, bmt3⁻, bmt4⁻, mnn4⁻, pno1⁻, and mnn4L1⁻ (mnn4A⁻). In an embodiment of the invention, the host cell is (2) all of the above plus expresses a mannosidase 1B activity and GlcNAc transferase I activity. In an embodiment of the invention, the host cell is (3) all of the above wherein it expresses a mouse mannosidase 1B and/or human GlcNAc transferase I. In an embodiment of the invention, the host cell (4) incorporates any one, two or three of the previous embodiment characteristics plus expresses a mannosidase II activity and/or a GlcNAc transferase II activity. In an embodiment of the invention, the host cell (5) incorporates any one, two, three or four of the previous embodiment characteristics wherein it expresses a Drosophila mannosidase II and/or a rat GlcNAc transferase II. In an embodiment of the invention, the host cell (6) incorporates any one, two, three, four or five of the previous embodiment characteristics plus expresses a galactosyl transferase activity. In an embodiment of the invention, the host cell (7) incorporates any one, two, three, four, five or six of the previous embodiment characteristics wherein it expresses a human galactosyl transferase, a yeast UDP-Galactose C4-Epimerase and a Drosophila UDP-galactose transporter—in such a strain, a pmt2⁻, och1⁻ or pmt2⁻, och1⁻, pmt5⁻ mutant would allow for the production of antibodies, antibody fragments or other glycoproteins with terminal beta-1,4-galactose with reduced O-glycosylation; methods of using such a strain for this purpose are within the scope of the present invention, see, e.g., the protein expression section herein. In an embodiment of the invention, the host cell (8) incorporates any one, two, three, four, five, six or seven of the previous embodiment characteristics plus heterologously expresses the pathway to convert UDP-GlcNAc into CMP-sialic acid as well as a CMP-sialic acid golgi transporter and sialyl transferase—in such a strain, a pmt2⁻, och1⁻ or pmt2⁻, och1⁻, pmt5⁻ mutant would allow for the production of antibodies, antibody fragments or other glycoproteins with terminal sialic acids including alpha-2,3- and alpha-2,6-linked NANA with reduced O-glycosylation; methods of using such a strain for this purpose are within the scope of the present invention, see, e.g., the protein expression section herein. In an embodiment of the invention, the host cell (9) incorporates any one, two, three, four, five, six, seven, or eight of the previous embodiment characteristics plus heterologously expresses a parasite oligosaccharyl transferase subunit homolog; such a host cell would allow for minimizing O-glycosylation while maximizing occupancy at consensus N-linked glycan sites, e.g., the host cell in (9) heterologously expresses the Leishmania major STT3D oligosaccharyl transferase subunit homolog. In an embodiment of the invention, the host cell (10) incorporates any one, two, three, four, five, six, seven, eight or nine of the previous embodiment characteristics plus it has a mutant or deleted alg3 (core alpha-1,3-mannosyltransferase) gene. In an embodiment of the invention, the host cell in (10) is an alg3⁻ strain and expresses an endomannosidase activity.

In an embodiment of the invention, any secreted protein that lacks consensus N-glycosylation sites, but where an och1⁻ mutation is desirable and reduction of O-glycosylation is desired, can be expressed in such an pmt2⁻, och1⁻ or pmt2⁻, och1⁻, pmt5⁻ mutant as described herein for N-glycosylated proteins. For example, in an embodiment of the invention, an antibody or antigen-binding fragment thereof, where the N-297 consensus glycosylation site has been mutated to alanine, glutamine or any other amino acid that will not support N-glycosylation, can be expressed in an pmt2⁻, och1⁻ or pmt2⁻, och1⁻, pmt5⁻ strain to maximize secretion and at the same time reduce O-glycosylation, as described herein for natively N-glycosylated antibodies. In another embodiment, a natively non-N-glycosylated but secreted protein, such as human serum albumin, where reduction of O-glycosylation is desired, can be expressed in an pmt2⁻, och1⁻ or pmt2⁻, och1⁻, pmt5⁻ strain as described herein for N-glycosylated proteins.

As used herein, the term “essentially free of” as it relates to lack of a particular sugar residue, such as fucose, or galactose or the like, on a glycoprotein, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures. For example, in an embodiment of the present invention, glycoprotein compositions produced by host cells of the invention will “lack fucose,” because the cells do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

The present invention also includes an isolated Pichia cell comprising wild-type OCH1 polypeptide but partially or fully lacking functional PMT2 and/or PMT5 polypeptide, e.g., pmt2⁻, OCH1⁺ (e.g., wherein chromosomal PMT2 is mutated or partially or fully deleted or disrupted or PMT2 expression is reduced, for example, through use of siRNA or RNAi), as well as methods of use thereof, such as methods for expressing a heterologous polypeptide (e.g., an immunoglobulin) which are analogous to those discussed herein in connection with pmt2⁻, och1⁻ or pmt2⁻, och1⁻ pmt5⁻ cells.

### Protein Expression

The scope of the present invention includes methods for producing one or more heterologous polypeptides comprising (i) introducing a polynucleotide encoding the heterologous polypeptide(s) into a pmt2⁻, och1⁻ or pmt2⁻, och1⁻, pmt5⁻ fungal or lower eukaryotic host cell (e.g., a Pichia cell such as a Pichia pastoris cell, e.g., as discussed herein) and (ii) culturing the host cell under conditions favorable to expression of the heterologous polypeptide(s) in the cell (e.g., in a bioreactor or fermentor), for example, for as long as the cells are viable, and, optionally, (iii) isolating the heterologous polypeptide(s) from the host cell. Methods for expressing heterologous polypeptides in Pichia host cells are generally known and conventional in the art.

The present invention encompasses any isolated fungal or lower eukaryotic host cell, e.g., Pichia host cell (e.g., pmt2⁻, och1⁻ or pmt2⁻, och1⁻ pmt5), discussed herein suspended in a liquid culture medium. Any lysate of an isolated fungal or lower eukaryotic host cell, e.g., Pichia host cell, discussed herein is also within the scope of the present invention.

The culture conditions used for a fungal or lower eukaryotic host cell expression system can be varied depending on the particular conditions at hand. In an embodiment of the invention, fungal or lower eukaryotic host cells can be grown in liquid culture medium in shaken-flasks or in fermentors (e.g., 1 L, 2 L, 5 L, 10 L, 20 L, 30 L, 50 L, 100 L, 200 L, 500 L, 1000 L, 10,000 L volume). Various growth mediums may be used to culture fungal or lower eukaryotic host cells. In an embodiment of the invention, the medium is at a pH of between pH 3 and 6 (e.g., 3, 4, 5 or 6); in an embodiment of the invention, pH is increased with a base such as ammonium hydroxide. In an embodiment of the invention, the temperature is maintained at about 24° C. In an embodiment of the invention, dissolved oxygen in the growth medium is maintained at about 20% or 30%. In an embodiment of the invention, the growth medium contains yeast nitrogen base (e.g., with ammonium sulfate; with or without essential amino acids), peptone and/or yeast extract. Various supplements may be added to an growth medium such as biotin, dextrose, methanol, glycerol, casamino acids, L-arginine-hydrochloride, ammonium ions (e.g., in the form of ammonium phosphates). In an embodiment of the invention, the growth medium is minimal medium containing yeast nitrogen base, water, a carbon source such as dextrose, methanol or glycerol, biotin and histidine. In an embodiment of the invention, the cell culture comprises trace minerals/nutrients such as copper, iodine, manganese, molybdenum, boron, cobalt, zinc, iron, biotin and/or sulfur, e.g., CuSO₄, NaI, MnSO₄, Na₂MoO₄, H₃BO₃, CoCl₂, ZnCl₂, FeSO₄, biotin and/or H₂SO₄. In an embodiment of the invention, the cell culture comprises an anti-foaming agent (e.g., silicone).

The present invention encompasses methods for making a heterologous polypeptide (e.g., an immunoglobulin chain or an antibody or antigen-binding fragment thereof) comprising introducing, into an isolated fungal or lower eukaryotic pmt2⁻, och1⁻ or pmt2⁻, och1⁻, pmt5⁻ host cell (e.g., Pichia, such as Pichia pastoris), a heterologous polynucleotide encoding said polypeptide, e.g., that is operably linked to a promoter, e.g., a methanol-inducible promoter and culturing the host cells,

(i) in a batch phase (e.g., a glycerol batch phase) wherein the cells are grown with a non-fermentable carbon source, such as glycerol, e.g., until the non-fermentable carbon source is exhausted;
(ii) in a batch-fed phase (e.g., a glycerol batch-fed phase) wherein additional non-fermentable carbon source (e.g., glycerol) is fed, e.g., at a growth limiting rate; and
(iii) in a methanol fed-batch phase wherein the cells are grown in the presence of methanol and, optionally, additional glycerol.

In an embodiment of the invention, in the methanol fed-batch phase, methanol concentration is set to about 2 grams methanol/liter to about 5 grams methanol/liter (e.g., 2, 2.5, 3, 3.5, 4, 4.5 or 5).

In an embodiment of the invention, prior to the batch phase, an initial seed culture is grown to a high density (e.g., OD₆₀₀ of about 2 or higher) and the cells grown in the seed culture are used to inoculate the initial batch phase culture medium.

In an embodiment of the invention, after the batch-fed phase and before the methanol fed-batch phase, the host cells are grown in a transitional phase wherein cells are grown in the presence of about 2 ml methanol per liter of culture. For example, the cells can be grown in the transitional phase until the methanol concentration reaches about zero.

Heterologous polypeptides that are isolated from a fungal or lower eukaryotic host cell are, in an embodiment of the invention, purified. If the heterologous polypeptide is secreted from the fungal or lower eukaryotic host cell into the liquid growth medium, the polypeptide can be purified by a process including removal of the fungal or lower eukaryotic host cells from the growth medium. Removal of the cells from the medium may be performed using centrifugation, discarding the cells and retention of the liquid medium supernatant. If the heterologous polypeptide is not secreted, the liquid medium can be discarded after separation from the fungal or lower eukaryotic host cells which are retained. Thereafter, the fungal or lower eukaryotic host cells may be lysed to produce a crude cell lysate from which the heterologous polypeptide may be further purified.

Heterologous polypeptide purification is, in an embodiment of the invention, performed by chromatography, e.g., column chromatography. Chromatographic purification can include the use of ion exchange, e.g., anion exchange and/or cation exchange, protein-A chromatography, size exclusion chromatography and/or hydrophobic interaction chromatography. Purification can also include viral inactivation of the composition comprising the polypeptide, precipitation and/or lyophilization.

### EXAMPLES

This section is intended to further describe the present invention and should not be construed to further limit the invention. Any composition or method set forth herein constitutes part of the present invention.

### Example 1

### Generation of a pmt2 Deletion Strain in an och1 Deletion Background by Conditional Allelic Replacement Using a Methanol-Dependent PMT2 Allele

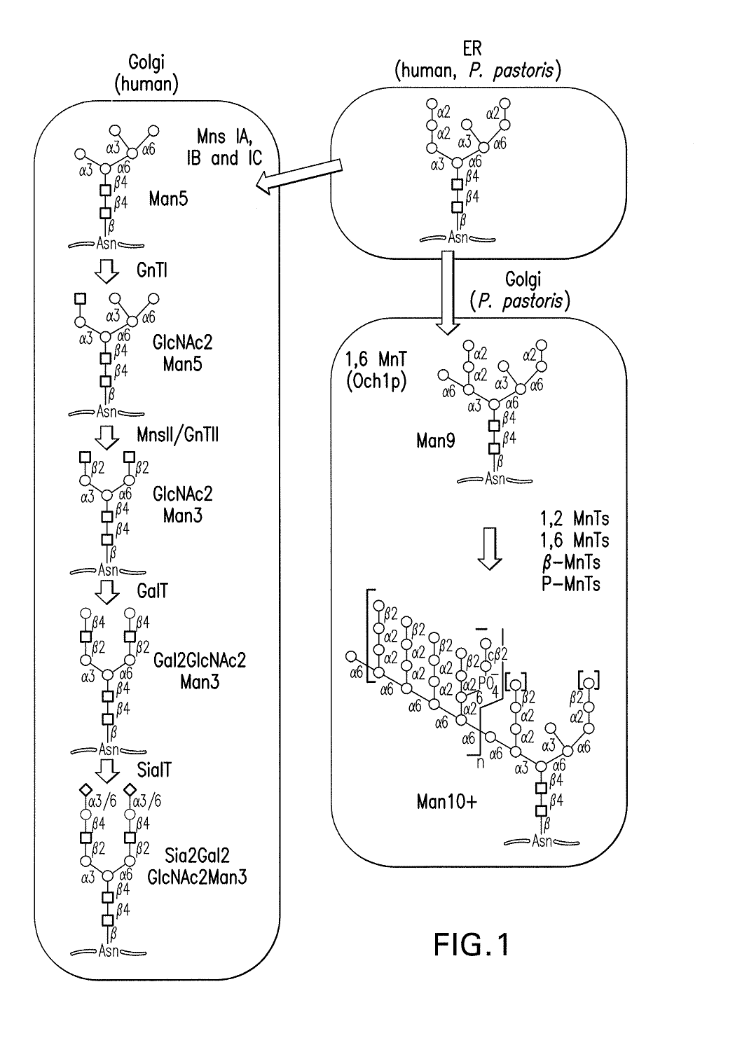

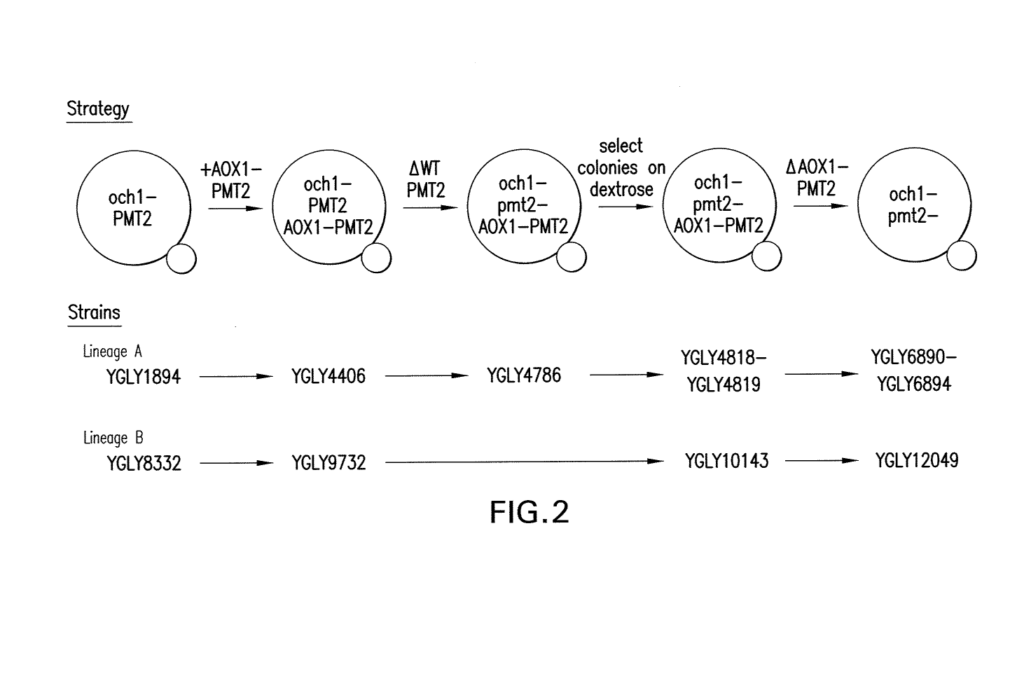

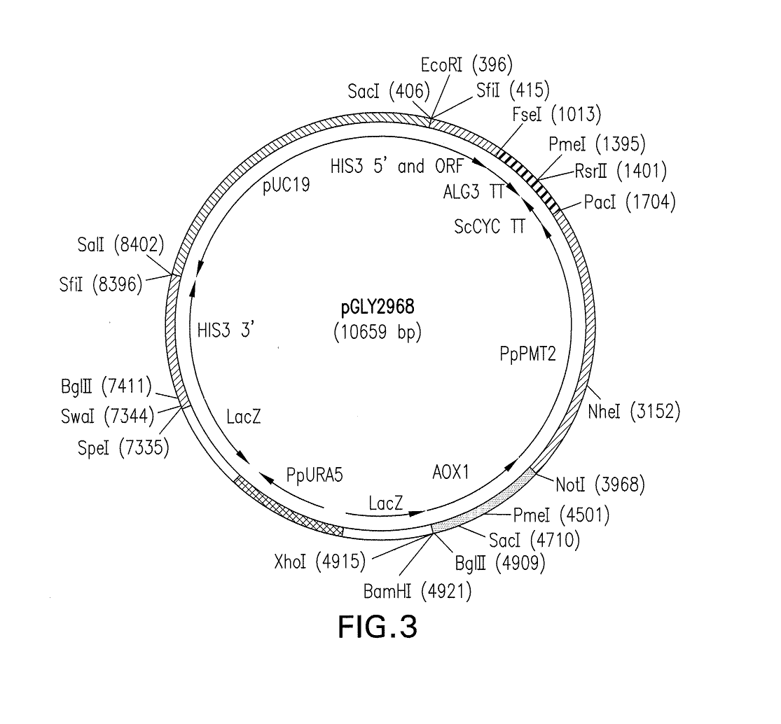

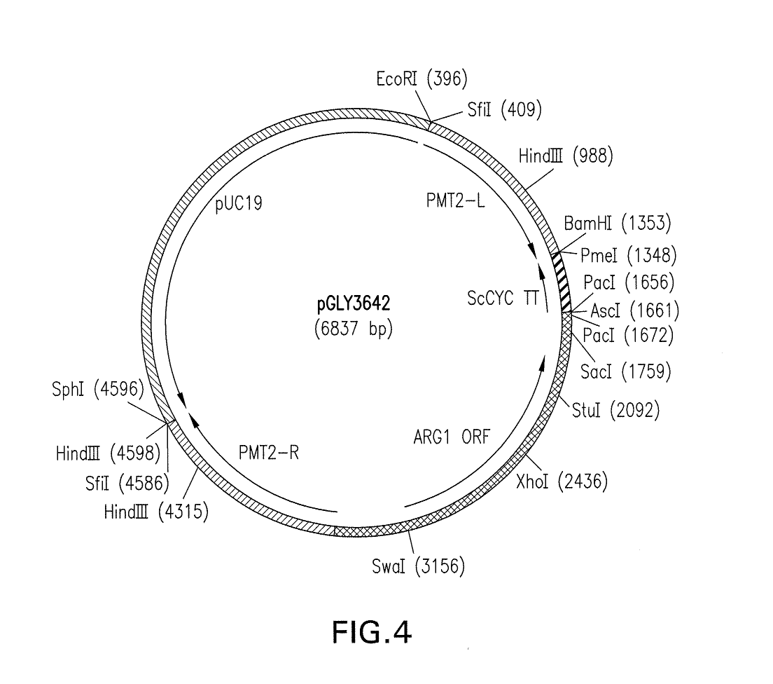

P. pastoris strains were previously engineered to secrete proteins with human N-glycans via deletion of och1 and several other key P. pastoris genes and expression of the mammalian mannosidase and glycosyl transferase genes necessary for assembly of the various desired human glycoforms (FIG. 1). It has become clear that assembly of monoclonal antibodies secreted by these strains is hindered by transfer of O-mannose performed by the protein O-mannosyl transferase (PMT) genes (Published International Patent Application No. WO07061631, Kuroda et al). Despite this knowledge, to date, deletion of the PMT2 gene has been unsuccessful in N-glycan modified strain backgrounds, including och1 deletion background and in human N-glycan producing glycoengineered P. pastoris strains. To generate a PMT2 knockout in an och1 mutant glycoengineered strain, conditional allelic replacement screening strategy was employed (FIG. 2). First an AOX1-driven allele of the PMT2 gene was generated. Plasmid pGLY2968 (FIG. 3) was constructed by inserting the AOX1 promoter from pGLY2269 and the PMT2 gene from pGLY2574 into the HIS3::URA5 targeted knock-in plasmid pGLY579. This plasmid was transformed into the ura5⁻ arg1⁻ double auxotrophic GFI5.0 glycoengineered strain YGLY1894 (FIG. 2). Strain YGLY1894 was previously engineered to secrete proteins with human N-glycans containing terminal β-1,4-galactose (U.S. Pat. No. 7,795,002). Clones from this transformation were selected on medium lacking uracil, and then confirmed by PCR primers specific for the HIS3 locus to generate strain YGLY4406. This strain was then transformed with plasmid pGLY3642, a standard knockout plasmid containing a pmt2::ARG1 allele, and digested with SfiI (FIG. 4). Clones were selected on medium lacking arginine but containing methanol as the sole carbon source to maintain expression of the AOX1-driven copy of PMT2. Positive knockout strains were confirmed by PCR for the PMT2 locus and one such strain was named YGLY4786. YGLY4786 was then cultivated in liquid medium containing methanol for 72 hours and plated to medium containing dextrose to select for colonies that could survive without expression of the AOX1-driven copy of PMT2. Two positive clones were identified and named YGLY4818 and YGLY4819.

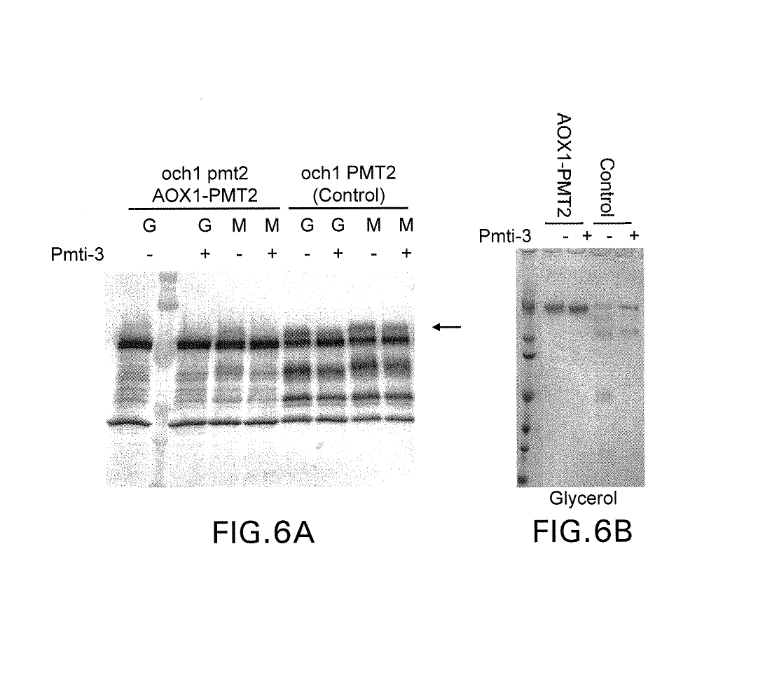

Strains YGLY4818 and YGLY4819 (along with a sister clone that yielded PMT2⁺ PCR results, YGLY4717) were transformed with plasmid pGLY4078, a plasmid containing GAPDH-promoter driven heavy chain and light chain genes for an IgG1 antibody targeting human CD20. Clones were cultivated in 96 well plate format (Barnard et al, 2010) in glycerol as a carbon source for 72 hours followed by a 24 hour cultivation in dextrose as a sole carbon source (to maximize mAb expression). No PMTi O-glycosylation inhibitor was added to the culture. Culture supernatants were harvested by centrifugation and subjected to protein A purification and SDS-PAGE and coomassie stain analysis. As shown in FIG. 5, clones from strain YGLY4717 produced poorly assembled antibody whereas those clones from strains YGLY4818 and YGLY4819 produced well assembled and intact antibody with no visible degraded fragments. A representative clone from YGLY4818, named YGLY5849, was cultivated in shake flasks along with a control strain, YGLY5771, which is a GFI5.0 PMT2⁺ anti-CD20-expressing strain. Shake flask cultivations were performed by first cultivating the strains in 50 ml of media with glycerol as the sole carbon source, then splitting the culture in two parts, centrifuging the cells and cultivating for 24 hours in 12 ml of media with dextrose as the sole carbon source with and without PMTi-3 O-glycan inhibitor. Supernatants were harvested by centrifugation and mAb was purified by protein A using standard procedures. The protein was then subjected to SDS-PAGE and coomassie stain analysis and Western blot analysis using anti-H/L antibody (Thermo Fisher Scientific, Rockford, Ill.) as shown in FIG. 6. The YGLY5771 control strain derived protein was generally intact and well assembled in the presence of PMTi-3 inhibitor as has been reported previously (Published International Patent Application No. WO07061631) but in the absence of inhibitor was poorly assembled and with degraded forms apparent. However, the YGLY5849 pmt2⁻, och1⁻ glycoengineered strain (containing only an AOX1-PMT2 allele) derived protein was equally well assembled in the presence or absence of PMTi-3 inhibitor. Purified protein was also subjected to HPAEC-PAD quantitative O-glycan analysis (Stadheim et al). The YGLY5771 derived protein contained 4.5 mol of O-mannose per mAb in the presence of PMTi-3 inhibitor but 23 mol/mol in the absence of inhibitor, where as the YGLY5849-derived mAb contained less than 1 mol/mol of O-mannose irrespective of inhibitor (Table 1).

TABLE 1
Mannosylation in och1, pmt2, AOX-PMT2 strain YGLY5849 in
the presence or absence of PMT inhibitor

| | PMTi-3 | O-linked Ser/Thr | |
|---|---|---|---|
| Strain (description) | (5 ug/mL) | per Mab | % Man1 |
| YGLY5849 (AOX1- | − | 0.6 | 100 |
| PMT2) | | | |
| YGLY5849 (AOX1- | + | 0.7 | 87 |
| PMT2) | | | |
| YGLY5771 (control) | − | 23 | 59 |
| YGLY5771 (control) | + | 4.5 | 76 |

*Mannosylation was evaluated after cultivation on glycerol so that AOX1-driven expression of PMT2 was not induced.

### Example 2

### Bioreactor Cultivation of a mAb-Expressing Glycoengineered Pichia Pmt2 Deletion Strain in an och1 Deletion Background Generated by Conditional Allelic Replacement

Four GAPDH anti-CD20-expressing clones from YGLY4818 were cultivated in 0.5 L fermenters using the Infors multifermentation system (Barnard et al, 2010). These clones were compared to YGLY5771 and YGLY5772, two control GFI5.0 GAPDH-driven anti-CD20 producing strains. The process was modified from that used by Barnard et al to suit expression from the GAPDH promoter. Instead of a limited methanol feed during induction, cultures were fed with glucose in a limited feed following the standard glycerol batch phase. Furthermore, each of the fermentations was carried out in duplicate, both with and without addition of PMTi-3 O-glycosylation inhibitor. The data in Table 2 showed that, when the control strains were cultivated in the presence of PMTi-3, the O-mannose levels as measured by HPAEC-PAD were low, in the range of 1-5 mannose chains attached to Ser/Thr per mAb tetramer. On the other hand, occupancy of mannose is much higher in the absence of inhibitor, in the range of 35-45 occupied Ser/Thr residues. This value is consistent with historical data, including the high level of variability, which can range from 30-50 but is always at least an order of magnitude higher than cultivation in the presence of PMTi-3. The pmt2 knockout strains (containing only the repressed AOX1-PMT2 allele), conversely had low O-mannose occupancy in both the presence and absence of inhibitor, confirming the results from 96 well plate and shake flasks. Despite the fact that there are 5 PMT genes in Pichia, surprisingly, knockout of solely PMT2 is able to nearly eliminate O-mannose from secreted mAb. This indicates that the main target of the PMTi inhibitor is the Pmt2p protein. Another observation is that the residual of 1-5 O-mannose occupancy is likely the result of the activity of one or a combination of the other Pmtp proteins.

TABLE 2
Characterization of glycoengineered strain viability and monoclonal
antibody expression in various strains.

| Strain | PMTi-3 | O-linked Ser/Thr | Supt DNA | mAb titer |
|---|---|---|---|---|
| description | (5 ug/mL) | per Mab | (mg/L) | (mg/L) |
| och1 pmt2 | − | 2.2 +/− 0.2 | 9.7 | 118 +/− 10 |
| AOX1-PMT2 | | | | |
| (n = 4) | | | | |
| och1 pmt2 | + | 1.3 +/− 0.3 | 14.9 | 134 +/− 12 |
| AOX1-PMT2 | | | | |
| (n = 4) | | | | |
| och1 PMT2 | − | 39.0 +/− 9.1 | 8.9 | 13 +/− 0 |
| (control) (n = 2) | | | | |
| och1 PMT2 | + | 2.6 +/− 0.4 | 16.0 | 35 +/− 5 |
| (control) (n = 2) | | | | |

### Example 3

### Generation of a Complete pmt2 Deletion Strain in a Glycoengineered och1 Deletion Background by Conditional Allelic Replacement and Subsequent Elimination of the Conditional Allele

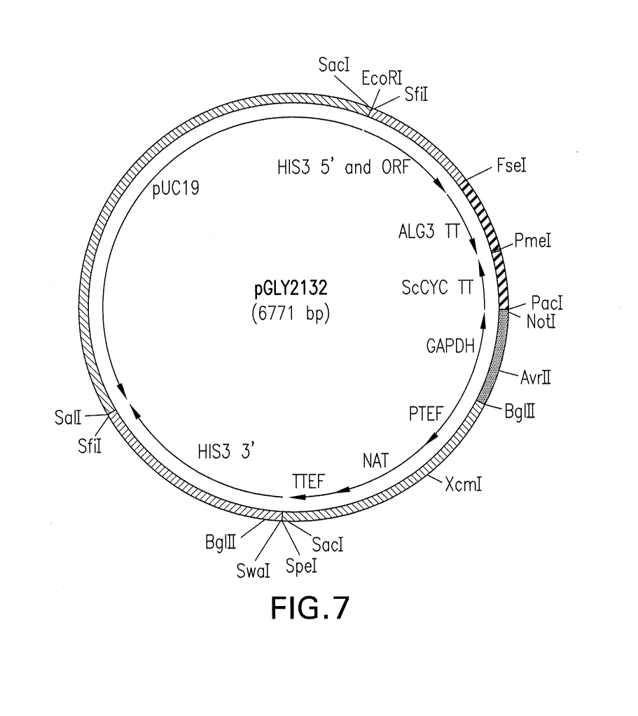

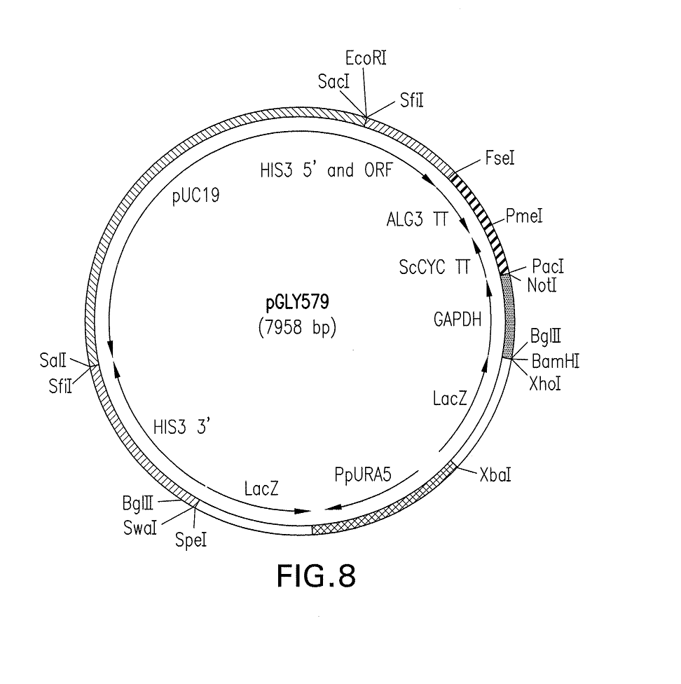

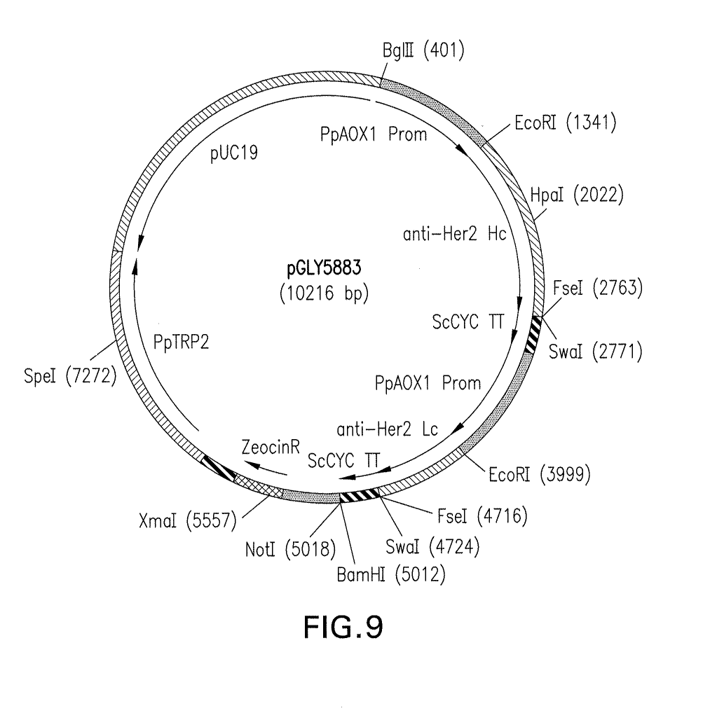

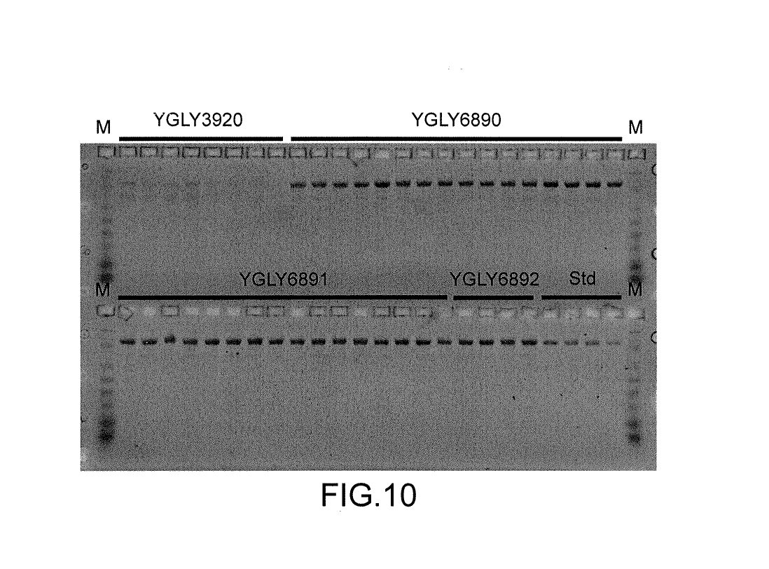

To confirm that the pmt2 knockout strains containing only the AOX1-PMT2 allele were able to survive in the complete absence of a PMT2 gene, AOX1-PMT2 allele was removed by transformation of strain YGLY4819 with pGLY2132 (FIG. 7), containing a HIS3::NAT allele that replaces the entire locus with the Nourseothricin resistance gene. Clones were selected on medium containing 100 μg/ml Nourseothricin as previously described (Goldstein et al, 1999). Positive clones were counter screened for uracil auxotrophy because proper integration of this plasmid will also eliminate the URA5 gene. The URA5 gene was then reintroduced into a positive clone using plasmid pGLY579 (FIG. 8) and positive clones were counterscreened for Nourseothricin sensitivity due to elimination of the NatR gene. Three positive complete pmt2 deletion strains were saved and named YGLY6890, YGLY6891, and YGLY6892. These strains are prototrophic and lack both the genomic and AOX1-driven copies of PMT2. To determine whether these strain would have reduced O-mannose, pGLY5883 (FIG. 9), a construct containing the genes encoding the anti-HER2 monoclonal antibody heavy and light chains driven by the AOX1 promoter was introduced and selected for by resistance to Zeocin. Positive clones, confirmed by positive growth on Zeocin containing medium, were cultivated along with YGLY3920, a PMT2 wild type control strain that produces an anti-CD20 mAb also under control of the AOX1 promoter, in 96 well plate format in glycerol, followed by induction on methanol as a sole carbon source (Barnard et al, 2010). Cultivations were performed in the absence of PMTi-3/PMTi-4 O-mannose inhibitor. Supernatant from these cultivations was purified by protein A-based bead assay and separated on SDS-PAGE followed by coomassie stain (Barnard et al, 2010). As shown in FIG. 10, the pmt2 knockout strain-derived clones produced significantly more and better assembled mAb than the control PMT2 wild type strain.

### Example 4

### Bioreactor Cultivation of a Complete Pmt2 Deletion Strain in an och1 Deletion Background

An AOX1-driven allele of the PMT2 gene was introduced into the ura5⁻ arg1⁻ double auxotrophic GFI5.0 glycoengineered strain YGLY8332 (FIG. 2) a parallel lineage but identical strategy to that described for strain YGLY1894 in Example 1, by transformation of plasmid pGLY2968. Clones from this transformation were selected on medium lacking uracil, and then confirmed by PCR primers specific for the HIS3 locus to generate strain YGLY9732. This strain was then transformed with plasmid pGLY3642 a standard knockout plasmid containing a pmt2::ARG1 allele, digested with SfiI (FIG. 4). Clones were selected on medium lacking arginine but containing methanol as the sole carbon source to maintain expression of the AOX1-driven copy of PMT2. Positive knockout strains were confirmed by PCR for the PMT2 locus and then adapted for growth on dextrose by cultivation in liquid medium containing methanol for 72 hours and selection on solid dextrose containing. One positive pmt2 knockout clone was identified that was capable of robust growth on dextrose and was named YGLY10143. To confirm that the pmt2 knockout strains containing only the AOX1-PMT2 allele were able to survive in the complete absence of a PMT2 gene, the AOX1-PMT2 allele was removed by transformation of pGLY2132 (FIG. 7), containing a HIS3::NAT allele that replaces the entire locus with the Nourseothricin resistance gene. Clones were selected on medium containing 100 μg/ml Nourseothricin as previously described (Goldstein et al., 1999). Positive clones were counter screened for uracil auxotrophy because proper integration of this plasmid will also eliminate the URA5 gene. The URA5 gene was then reintroduced into a positive clone using plasmid pGLY579 (FIG. 8) and positive clones were counterscreened for Nourseothricin sensitivity due to elimination of the NatR gene. One positive complete pmt2 deletion strain was saved and named YGLY12049. This strain is prototrophic and lacks both the genomic and AOX1-driven copies of PMT2. To determine whether this strain would have reduced O-mannose, pGLY5883, a construct containing the genes encoding the anti-HER2 monoclonal antibody heavy and light chains driven by the AOX1 promoter was introduced and selected for by resistance to Zeocin (FIG. 9). One such anti-HER2 expressing clone from YGLY12049, named YGLY14564, was cultivated in an 0.5 L fermenter and compared to a lead anti-HER2 expressing strain, YGLY13979, in a similar GFI5.0 background that contains the wild type PMT2 gene. As shown in Table 3, the pmt2 deletion strain was able to produce anti-HER2 mAb with significantly reduced O-mannose (5.9 vs. 47.3 mol/mol of mAb) compared to the lead PMT2 wild type strain in the absence of PMTi-3 inhibitor, as measured by HPAEC-PAD. Again, the PMT2 wild type control (YGLY13979) exhibited the historically expected degree of O-mannosylation (30-50 mol/mol) in the absence of inhibitor while the pmt2 knockout strain produced mAb with unexpectedly reduced O-mannose, comparable to a strain cultivated in the presence of PMTi-3 or PMTi-4 O-mannose inhibitor. The lysis of the pmt2 deletion strain was also significantly lower, which might be an indication of the reduced stress of producing misfolded and degraded mAb fragments. While the titer of the YGLY13979 was higher than that of the YGLY14564 pmt2 knockout strain, this control strain was screened from among thousands of potential clones for the highest titer, while YGLY14564 was not and this HPLC-based titer method does not distinguish between mAb fragments and fully assembled mAb tetramer.

TABLE 3
Characterization of glycoengineered strain viability and anti-HER2
monoclonal antibody expression in an och1 strain with and without PMT2.

| Strain | PMTi-3 | O-linked Ser/Thr | Supt DNA | mAb titer |
|---|---|---|---|---|
| description | (5 ug/mL) | per Mab | (mg/L) | (mg/L) |
| och1 pmt2 | − | 5.9 | 0.5 | 194 |
| (YGLY14564 ) | | | | |
| och1 PMT2 | − | 47.3 | 20.5 | 310 |
| (YGLY13979) | | | | |

### Example 5

### Knockout of Pmt2 in an och1 Deletion Background Using a Cre/Lox Recombination Approach

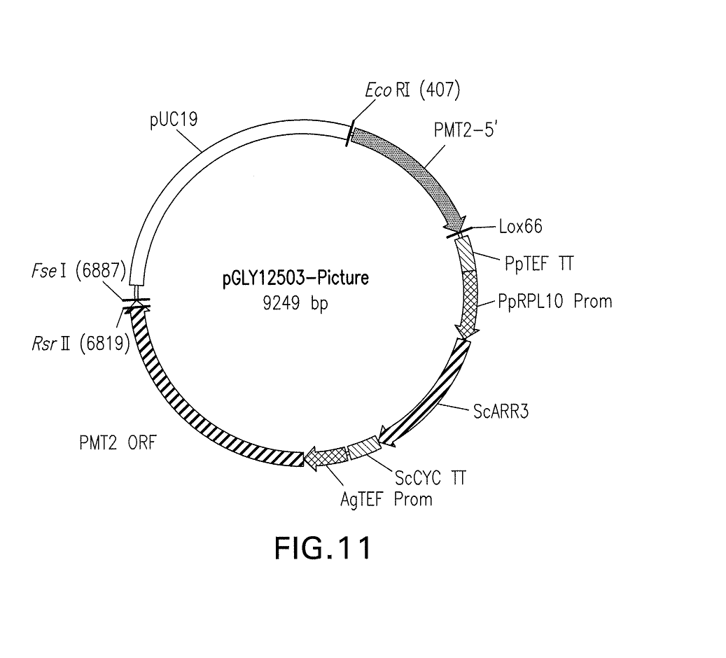

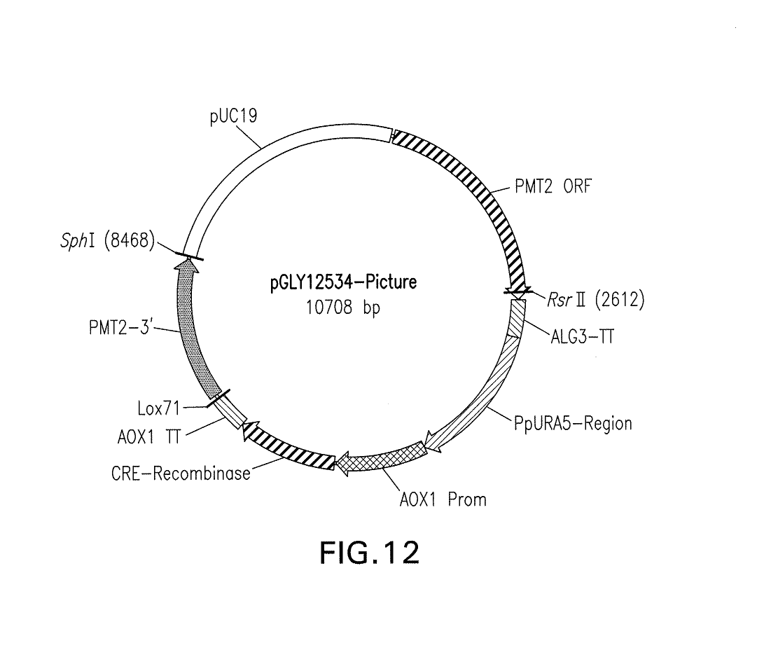

To generate a linear Cre-LoxP PMT2 DNA replacement allele, plasmid pGLY12503 was digested with EcoRI and FseI restriction enzymes. The 407 bp-6887 bp fragment of pGLY12503 (FIG. 11) was isolated by gel electrophoresis and purified. Similarly, plasmid pGLY12534 (FIG. 12) was digested with RsrII and SphI restriction enzymes; the 2612 bp-8468 bp fragment was gel separated and purified. The two DNA fragments have 68 bp of overlapping sequence identity. The two digested and isolated DNA fragments were combined and used as templates for the following fusion PCR reaction to generate the linear Cre-LoxP PMT2 replacement allele. The fusion PCR reaction uses primers PMT2-KO-5UTR-FW2 (5′-ATTGTCAACGAAGTTGTTGGAGTTAAGAC-3′) (SEQ ID NO: 5) and PMT2-KO-3UTR-RV2 (5′-TTTCTGTTCATTTTCTCCAGAAGCTATGTCTC) (SEQ ID NO: 6). The PCR conditions were one cycle of 94° C. for 2 minutes, 25 cycles of 94° C. for 15 seconds, 58° C. for 30 seconds, and 68° C. for 14 minutes; followed by one cycle of 68° C. for 14 minutes. The fusion PCR generates a 12.2 kb linear DNA fragment.

Yeast strain YGLY27983 was used as the parental strain of the following example. The construction of yeast strain YGLY13979 has been disclosed in U.S. Patent Application Number US2010/0025211. Strain YGLY27983 was selected from strain YGLY13979 derivatives and is considered to be an isogenic sister clone of strain YGLY13979. The strain produces an anti-HER2 antibody with GS5.0 N-glycan structure (FIG. 1). In this strain, the expression cassettes encoding the anti-Her2 heavy and light chains are targeted to the Pichia pastoris TRP2 locus (PpTRP2). This strain contains the wild-type PMT2 sequence.

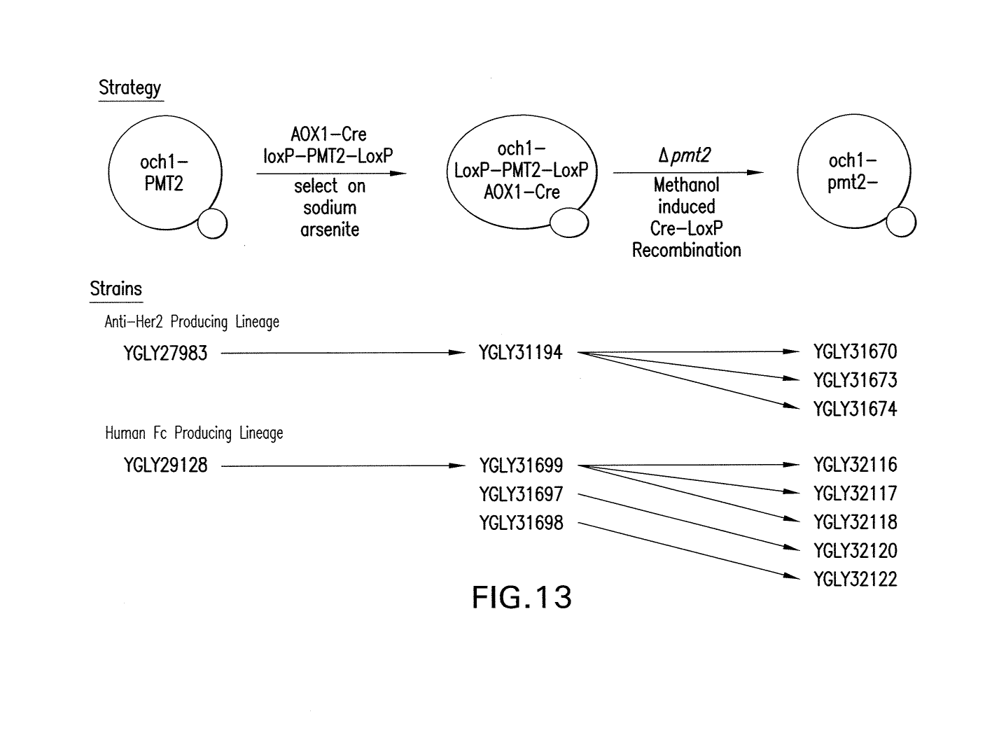

The 12.2 kb fusion PCR product was transformed into the P. pastoris strain YGLY27983 to produce PMT2 replacement strain YGLY31194 (i.e., Cre-LoxP flanking the endogenous PMT2 locus; FIG. 13). The transformants were selected on 0.2 mM sodium arsenite YSD plates. The genomic integration at the PMT2 locus was confirmed by cPCR using the primers, PpPMT2-A (5′-AAGAAGCGTTGTAGCTGGAAGAGCA-3′; SEQ ID NO: 7) and PpRPL10-Prom-RV (5′-GAGCAAAATCGAGAAGGTAGTGCATCA-3′; SEQ ID NO: 8) or PpPMT2-B (5′-GAGTAAAACCAATTATCCCTGGGCTTTAG-3′; SEQ ID NO: 9) and AOX1-TT-FW (5′-AAAACTATGTGGCAAGCCAAGC-3′; SEQ ID NO: 10). The PCR conditions were one cycle of 94° C. for 30 seconds, 30 cycles of 94° C. for 20 seconds, 55° C. for 30 seconds, and 72° C. for 2 minutes; followed by one cycle of 72° C. for 5 minutes. The Cre gene was linked to the AOX1 promoter.

To induce PMT2 Knock-out using Cre-LoxP recombination, strain YGLY31194 was cultivated in the presence of methanol in 10 mL BMMY (buffered methanol complex medium, Invitrogen, a division of Life Technologies, Carlsbad, Calif.) media in a 50 mL shake flask overnight, to induce expression of the AOX1promoter-Cre recombinase allele. Afterwards, cells were serially diluted and plated to form single colony on YSD plates. The strains YGLY31670, YGLY31673, and YGLY31674 were selected from the strains produced. Loss of genomic PMT2 sequences was confirmed using cPCR primers, PpPMT2-C (5′-ACGTTAAAATGAGGTTATTCAATGCCACC-3′ (SEQ ID NO: 11) and PpPMT2-D (5′-CACCGGTACCAGAATTGGATAATATTTCAA-3′ (SEQ ID NO: 12). The PCR conditions were one cycle of 94° C. for 30 seconds, 30 cycles of 94° C. for 20 seconds, 55° C. for 30 seconds, and 72° C. for 30 seconds; followed by one cycle of 72° C. for 1 minute.

### Example 6

### Engineered pmt2Δ Strains Display Improved mAb Yield and Protein Quality Under Fermentation Conditions

Yeast strains were cultivated in a DasGip 1 Liter fermentor without PMTi-4 O-mannose inhibitor to produce the antibodies for titer and protein quality analyses. Cell growth conditions of the transformed strains for antibody production in the DasGip fermentor were generally as follows: The seed flasks were inoculated from yeast patches (isolated from a single colony) on agar plates into 0.1 L of 4% BSGY in a 0.5-L baffled flask. Seed flasks were grown at 180 rpm and 24° C. (Innova 44, New Brunswick Scientific) for 48 hours. Fed-batch fermentation was done in 1-L (fedbatch-pro, DASGIP BioTools) bioreactors. Inoculation of a prepared bioreactor occurred aseptically with 60 mL from a seed flask. Vessels were charged with 0.54 L of 0.2 μm filtered 4% BSGY media (with 4 drops/L Sigma 204 antifoam) and autoclaved at 121° C. for 60 minutes. After sterilization and cooling; the aeration, agitation and temperatures were set to 0.7 vvm, 640 rpm and 24° C. respectively. The pH was adjusted to and controlled at 6.5 using 30% ammonium hydroxide. Agitation was ramped to maintain 20% dissolved oxygen (DO) saturation. DasGip fermentor screening protocol followed the parameters listed below: 4% BSGY-M: 40 g/L glycerol, 20 g/L soytone, 10 g/L yeast extract, 11.9 g/L KH₂PO₄, 2.3 g/L K₂HPO₄, 50 g/L maltitol, 13.4 g/L YNB with ammonium sulfate without amino acids, 8 mg/L Biotin. PTM2 salts: 0.6 g/L CuSO₄-5H₂O, 80 mg/L NaI, 1.8 g/L MnSO₄—H₂O, 20 mg/L H₃BO₄, 6.5 g/L FeSO₄-7H₂O, 2.0 g/L ZnCl₂, 0.5 g/L CoCl₂-6H₂O, 0.2 g/L Na₂MoO₄-2H₂O, 0.2 g/L biotin, 5 mL/L H₂SO₄ (85%). After the initial glycerol charge was consumed, denoted by a sharp increase in the dissolved oxygen, a 50% w/w glycerol solution containing 5 mg/L biotin and was triggered to feed at 3.68 mL/hr for 8 hours. During the glycerol fed-batch phase 0.375 mL of PTM2 salts were injected manually. Completion of the glycerol fed-batch was followed by a 0.5 hour starvation period and initiation of the induction phase. A continuous feed of a 50% v/v methanol solution containing 2.5 mg/L biotin and 6.25 mL/L PTM2 salts was started at a flat rate of 2.16 mL/hr. Individual fermentations were harvested within 36-110 hours of induction depending upon the durability of the strain. The culture broth was clarified by centrifugation (Sorvall Evolution RC, Thermo Scientific) at 8500 rpm for 40 minutes.

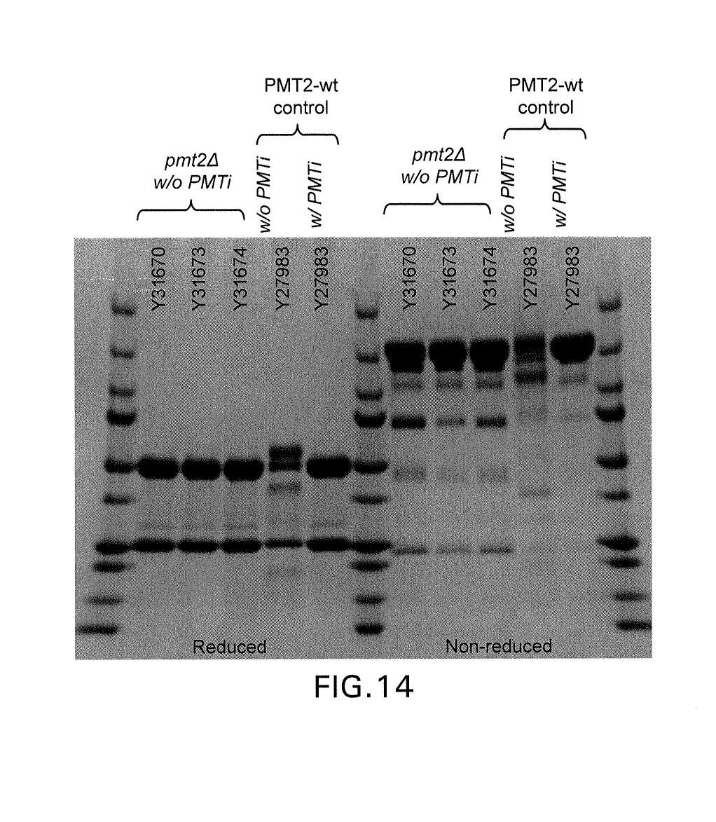

FIG. 14 shows the reducing and non-reducing SDS-PAGE for anti-HER2 material generated by pmt2Δ P. pastoris strains and their comparison with material generated by parental YGLY27983 (PMT2 wild-type, as described in Example 4) P. pastoris without or with PMT-i4 inhibitor. As shown in FIG. 14, the pmt2 knockout strain-derived clones produced significantly more and better assembled mAb than the control PMT2 wild type strain. As shown in Table 4, the YGLY27983 derived protein contained 1.1 mol of O-mannose per mAb in the presence of PMTi-4 inhibitor but 46.2 mol/mol in the absence of inhibitor, whereas the YGLY27983-derived mAb contained less than 1.5 mol/mol of O-mannose irrespective of inhibitor.

TABLE 4
Characterization of glycoengineered strain viability and anti-HER2
monoclonal antibody expression in an och1 strain with and without PMT2.

| | | | Supt | |
|---|---|---|---|---|
| | PMTi-3 | O-linked Ser/Thr | DNA | mAb titer |
| Strain description | (5 ug/mL) | per Mab | (mg/L) | (mg/L) |
| och1 pmt2 double | − | 1.48 +/− 0.03 | x | 330 |
| knockout (n = 4) | | | | |
| YGLY27983 och1 | − | 46.2 | x | 157 |
| PMT2 (control) (n = 1) | | | | |
| YGLY27983 och1 | + | 1.1 | x | 395 |
| PMT2 (control) (n = 1) | | | | |

### Example 7

### Knockout of PMT2 in a GFI6.0 Human Fc Producing Strain Reduces O-mannose

Human Fc producing strain YGLY29128 is used as the parental strain of this example. The strain produces the Fc region of human IgG with GS6.0 N-glycan structure (FIG. 1). In this strain, the expression cassette encoding the Fc region is targeted to the Pichia pastoris TRP2 locus (PpTRP2). This strain contains the wild-type PMT2 sequence. The pmt2Δ knock strains YGLY32116, YGLY32117, Y32118, Y32120, and YGLY32122 were generated from YGLY29128 using the cre-LoxP recombination methods as described in Example 5. Yeast strains were cultivated in a DasGip 1 Liter fermentor without PMTi-4 O-mannose inhibitor using a dissolved-oxygen limited fermentation protocol similar to methods as described in Example 6 to produce the Fc for titer and protein quality analyses. Under the oxygen limited fermentation condition, the agitation rate was locked at 640 rpm and a bolus addition of 6.8 mL of 100% methanol containing 5 mg/L biotin and 6.25 mg/L PTM2 salts was added. During methanol induction phase the DO remains at close to 0% until the methanol bolus is entirely consumed. Once the DO increases to >30% another 6.8 mL bolus of 100% methanol feed was added to prolong the induction time.

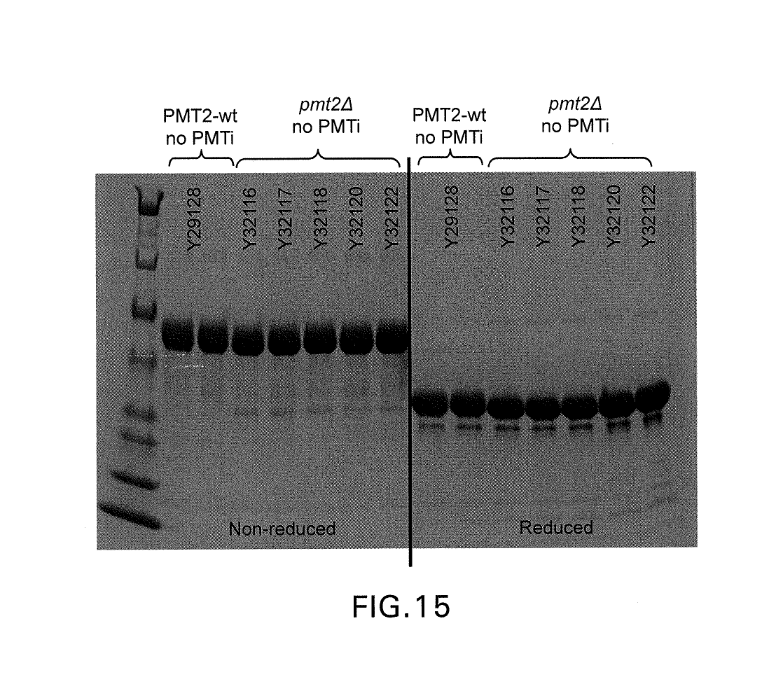

FIG. 15 shows the non-reducing and reducing SDS-PAGE for Fc material generated by pmt2Δ P. pastoris strains and their comparison with material generated by parental YGLY29128 (PMT2 wild-type) P. pastoris in the absence of PMT-i4 inhibitor. As shown in FIG. 15, the pmt2 knockout strain-derived clones produced better assembled Fc dimer than the control PMT2 wild type strain. As shown in Table 5, the YGLY29128 derived protein contained 3.91 mol of O-mannose per mAb in the absence of PMTi-4 inhibitor, whereas the YGLY29128-derived mAb contained 0.32 mol/mol of O-mannose irrespective of inhibitor, reducing O-mannose by more than 90%.

TABLE 5
Characterization of glycoengineered strain viability and Fc expression
in an och1 strain with and without PMT2.

| Strain | | O-linked Ser/Thr | Supt DNA | Fc titer |
|---|---|---|---|---|
| description | PMTi-4 | per Mab | (mg/L) | (mg/L) |
| och1 pmt2 | − | 3.91 | x | 1116 |
| double | | | | |
| knockout (n = 5) | | | | |
| YGLY29128 | − | 0.32 | x | 1020 |
| och1 PMT2 | | | | |
| (control) (n = 2) | | | | |

### Example 8

### Methods for N-Glycan Analysis

### Overview

N-glycans were analyzed by enzymatic release from the protein and then by Matrix-Assisted Laser Desorption/Ionization Time-of-Flight (MALDI-TOF) mass spectrometry and also by labeling with 2-amino benzamide and separation on reverse phase HPLC. First the glycans were released and separated from the glycoproteins by a modification of a previously reported method (Papac et al). The proteins were reduced and carboxymethylated, and the membranes blocked, then wells were washed three times with water. The protein was then enzymatically deglycosylated by the addition of 30 μl of 10 mM NH₄HCO₃ pH 8.3 containing one milliunit of N-glycanase (New England Biolabs, Ipswich, Mass.). After 16 hours at 37° C., the solution containing the glycans was removed by centrifugation and evaporated to dryness.

Molecular weights of the glycans were determined by using a Voyager DE PRO linear MALDI-TOF (Applied Biosciences) mass spectrometer with delayed extraction. The dried glycans from each well were dissolved in 15 μl of water, and 0.5 μl was spotted on stainless steel sample plates and mixed with 0.5 μl of S-DHB matrix (9 mg/ml of dihydroxybenzoic acid, 1 mg/ml of 5-methoxysalicilic acid in 1:1 water/acetonitrile 0.1% TFA) and allowed to dry. Ions were generated by irradiation with a pulsed nitrogen laser (337 nm) with a 4-ns pulse time. The instrument was operated in the delayed extraction mode with a 125 ns delay and an accelerating voltage of 20 kV. The grid voltage was 93.00%, guide wire voltage was 0.1%, the internal pressure was less than 5×10⁻⁷ torr, and the low mass gate was 875 daltons. Spectra were generated from the sum of 100 to 200 laser pulses and acquired with a 500 MHz digitizer. Man₅GlcNAc₂ oligosaccharide was used as an external molecular weight standard. All spectra were generated with the instrument in the positive ion mode.

2-Aminobenzamide (2-AB) labeling was used to quantify N-glycan structures. A solution of 5% 2-AB dye and 6.3% sodium cyanoborohydride was prepared in 1:4 glacial acetic acid/DMSO. Five microliters of this solution was added to dried glycan samples, mixed, and incubated for 2-3 h at 65° C. Each sample was applied to wells of a 96-well lysate plate (Promega Cat# A2241, Madison, Wis.) and then washed and pre-wetted with acetonitrile and adsorbed for 10-15 min; wells were then washed with 1 ml acetonitrile followed by three 1 ml 96% acetonitrile/4% water washes. Glycans were eluted three times with 0.4 ml water and dried in a centrifugal vacuum for 24 h. Labeled glycans were then separated by HPLC using a flow rate of 1.0 ml/min with a Prevail CHO ES 5-micron bead, amino-bound column using a 50-min linear gradient of 80% to 40% buffer A (100% acetonitrile). Buffer B consisted of 50 mM ammonium formate pH 4.4. Sialylated glycans were separated using a 30-min 80-40% Buffer A linear gradient with an additional 30-min gradient bringing buffer A from 40% to 0%. Labeled glycans were detected and quantified against standards using a fluorescence detector with an excitation of 330 nm and an emission at 420 nm.

### PNGase, MALDI-TOF, 2AB Labeling & HPLC Analysis of N-Glycans

### Release of N-Linked Glycans

Purpose: To describe the method for the release of N-linked glycans

### Materials:

RCM buffer (8M Urea, 360 mM Tris, 3.2 mM EDTA pH 8.6)

### 0.1M DTT (in RCM Buffer)

1% PVP 360 (in water)

### 10 mM NH₄HCO₃

Multiscreen 96-well plate, pore size 0.45 um (Millipore Cat# MAIPN4510, or equivalent)

### Methanol

### Summary of Method:

### Preparation of Sample

Add 100 μL DiH2O to each well of dried protein
Add 200 μL RCM buffer to each well of dried protein
*If sample is in aqueous solution, omit water and add 2× volume RCM buffer

### Addition and Reduction of Samples

Wet 96-well MultiScreen plate with 100 μl of methanol, and drain with gentle vacuum
Wash with 200 μl of RCM buffer, and drain with a gentle vacuum
Add 100 μL sample mixture and drain with gentle vacuum
Repeat until sample is fully loaded
Wash twice with RCM buffer (2×200 μL).
Add 50 μL 0.1 M DTT to reduce the proteins

### Incubate for 1 hr at 37° C.

### Block Membranes

Drain the wells by gentle vacuum
Wash the wells three times with 300 μL water
Add 100 μl of 1% PVP 360 to block membranes
Incubate for 1 hr at room temperature.

### Protein Deglycosylation

Drain the wells by gentle vacuum
Wash three times with 300 μl of HPLC grade water
Add 25-30 μl of 10 mM NH4HCO3 pH 8.3 containing one milliunit of N-glycanase (Glyko) or 10 unit of N-glycanase (GlycoFi)

### Incubate 16 hr at 37° C.

### Glycan Removal

Remove plate(s) from incubation and manually remove glycans from wells to a clean PCR plate
Alternately, glycans may be removed by centrifugation
Evaporate glycans to dryness

### Proceed to Analysis by Mass Spectrometry

*The glycans were released and separated from the glycoproteins by a modification of a previously reported method (Papac et al 1998). After the proteins were reduced and the membranes blocked, the wells were washed three times with water. The protein was deglycosylated by the addition of 30 μl of 10 mM NH₄HCO₃ pH 8.3 containing one milliunit of N-glycanase (Glyko) or 10 unit of N-glycanase (New England Biolab). After 16 hr at 37° C., the solution containing the glycans was removed by centrifugation and evaporated to dryness.

### General Conditions for MALDI-TOF

### Example of Instrument Settings: Positive Mode—

Mode of operation: Linear
Extraction mode: Delayed

### Polarity: Positive

Acquisition control: Manual
Accelerating voltage: 20000 V
Grid voltage: 92%
Guide wire 0: 0.05%
Extraction delay time: 100 nsec
Acquisition mass range: 850-3200 Da
Number of laser shots: 100/spectrum
Laser intensity: 1968

### Laser Rep Rate: 20.0 Hz

Calibration type: Default
Calibration matrix: 2,5-Dihydroxybenzoic acid
Low mass gate: 850 Da
Digitizer start time: 18.52
Bin size: 2 nsec
Number of data points: 8652
Vertical scale: 500 mV
Vertical offset: −2.5%
Input bandwidth: 150 MHz

### Negative Mode—

Mode of operation: Linear
Extraction mode: Delayed

### Polarity: Negative

Acquisition control: Manual
Accelerating voltage: 20000 V
Grid voltage: 94%
Guide wire 0: 0.08%
Extraction delay time: 225 nsec
Acquisition mass range: 1000-3500 Da
Number of laser shots: 100/spectrum
Laser intensity: 1990

### Laser Rep Rate: 20.0 Hz

Calibration type: Default
Calibration matrix: 2,5-Dihydroxybenzoic acid
Low mass gate: 800 Da
Digitizer start time: 20.144
Bin size: 2 nsec
Number of data points: 8716
Vertical scale: 500 mV
Vertical offset: −2.5%
Input bandwidth: 150 MHz

### iAB Protocol

Prep work:
Aliquot 100 μl of sample in a high-collar 96-well PCR plate and thoroughly mix with 100 μl of denature solution provided by the Prozyme i2AB labeling kit.
Set temperature on heat block to 50° C.
Prepare 1× reaction buffer (RX buffer). Create a reaction buffer master mix solution containing 4% 25× reaction buffer stock (supplied in Prozyme kit) and 96% HPLC grade water. To ensure enough reaction buffer is available to carry-out the protocol, allocate 150 μl of reaction buffer per sample.
Prepare reaction plate with the same number of reaction (RX) cartridges as samples being analyzed. Set cartridges on a collection plate (collection plate #1). Create a balance plate using used reaction or clean-up cartridges.

### Sample Addition and PNGase:

Wet reaction (RX) cartridge with 50 μl of 100% acetonitrile. Spin the plate at 300 g for 3 minutes. Discard flow through from collection plate #1.
Add 150 μl of denature reagent to the reaction cartridge and spin at 1000 g for 2 minutes.
Discard flow through.
Add sample-denature mixture to reaction cartridge. Dispense the sample carefully into the reaction cartridge to ensure there are no air pockets between the sample and the reaction cartridge membrane (any air pockets may hinder proper sample elution). Spin samples at 90 g for 10 minutes.
Some samples may not elute after that time. If so, spin the plate again at 1000 g for 1 minute until all sample wells have eluted.
Add 50 μl of blocking reagent (supplied by Prozyme kit) to cartridges. Spin at 300 g for 3 minutes. Discard flow though.
Add 100 μl of reaction buffer to cartridges. Spin at 300 g for 3 minutes. Discard flow through.
Replace the collection plate #1 with a clean collection plate (collection plate #2).
Prepare a master mix of PNGase reaction solution using the following ratio: 2.5 μl of PNGase to 7.5 μl reaction buffer (RX buffer) per cartridge.
Dispense 10 μl of the PNGase reaction solution to each reaction cartridge. Spin at 300 g for 3 minutes. Do not discard flow-through.
Fix entire reaction cartridge/collection plate set-up on the 50° C. heat block. Incubate for 30 minutes.
Remove plate from heat block and allow it to cool to room temperature. Add 20 μl of reaction buffer to the cartridges and spin at 300 g for 3 minutes to elute the glycans into collection plate #2.

### iAB Glycan Labeling:

### Dye Solution Prep:

Add 375 μl of dye solvent to 1 vial of dried instant 2AB dye (both supplied by Prozyme). In the absence of Prozyme-supplied dye solvent, DMSO also works as a solvent.

Mix solvent and instant 2AB dye thoroughly. After adding i2AB labeling reagents, store any remaining dye at −20° C.

Collection plate #2 should now contain about 30 μl of reaction buffer solution containing released glycans eluted off the reaction cartridges.
Remove the reaction cartridges from collection plate #2 and add 5 μl of i2AB labeling solution into each sample well on collection plate #2. Gently tap the plate to ensure the dye has made it to the bottom of the sample wells. Total volume should now be about 35 μl.
Add 215 μl of 100% acetonitrile into each sample well on collection plate #2. Mix well. A 250 μl solution of 86% acetonitrile is created when mixed with the 35 μl of labeled glycans.

### Clean-Up:

Prepare the clean-up plate with Prozyme clean-up cartridges and a new collection plate (collection plate #3).
Transfer 200 μl of labeled glycan solution from collection plate #2 into the clean-up cartridge. Transfer samples carefully to ensure no air pockets are formed between the sample and the clean-up cartridge membrane. Spin at 90 g for 10 minutes. Discard flow flow-through from collection plate #3.
Some samples may not elute after that time. If so, spin the plate again at 1000 g for 1 minute until all sample wells have eluted.
Add 200 μl of 96% acetonitrile to clean-up cartridge. Spin at 300 g for 3 minutes. Briefly spin again if any acetonitrile remains. Discard all flow through.

### Sample Elution, HPLC and Glycan Storage:

Replace collection plate #3 with a fresh collection plate (collection plate #4).
Add 50 μl of HPLC grade water to each clean-up cartridge. Spin at 300 g for 3 minutes.
Save eluted material. These are the labeled, cleaned-up glycans.
Mix eluted material well and aspirate 15 μl of material out of well and mix with 35 μl 100% acetonitrile in an HPLC tube. Set 10 μl per injection during HPLC run (see following page for HPLC conditions, HPLC column: Grace Prevail Carbohydrate ES5u 250 mm Cat No 35101).
Seal collection plate #4 and store the remaining labeled glycans at −20 C.

### HPLC Condition

### Agilent 1100/1200 Binary Pump:

Column Flow: 1.300 ml/min

### Stoptime: 45.00 min

### Posttime: Off

### Solvents:

### A=0.1 M Formic Acid pH 4.6

### B=100% ACN

### Timetable:

| Time | Solv.B | Flow |
|---|---|---|
| 0.00 | 70.0 | 1.300 |
| 20.00 | 56.0 | 1.300 |
| 35.00 | 0.0 | 1.300 |
| 38.00 | 0.0 | 1.300 |
| 38.05 | 70.0 | 1.300 |
| 45.00 | 70.0 | 1.300 |

### Agilent 1100/1200 Fluorescence Detector

### Signal:

### Excitation: 278 nm

### Emission: 344 nm

### PMT-Gain: 15

### Example 9

### Knockout of pmt5 Using Plasmid pGLY12527

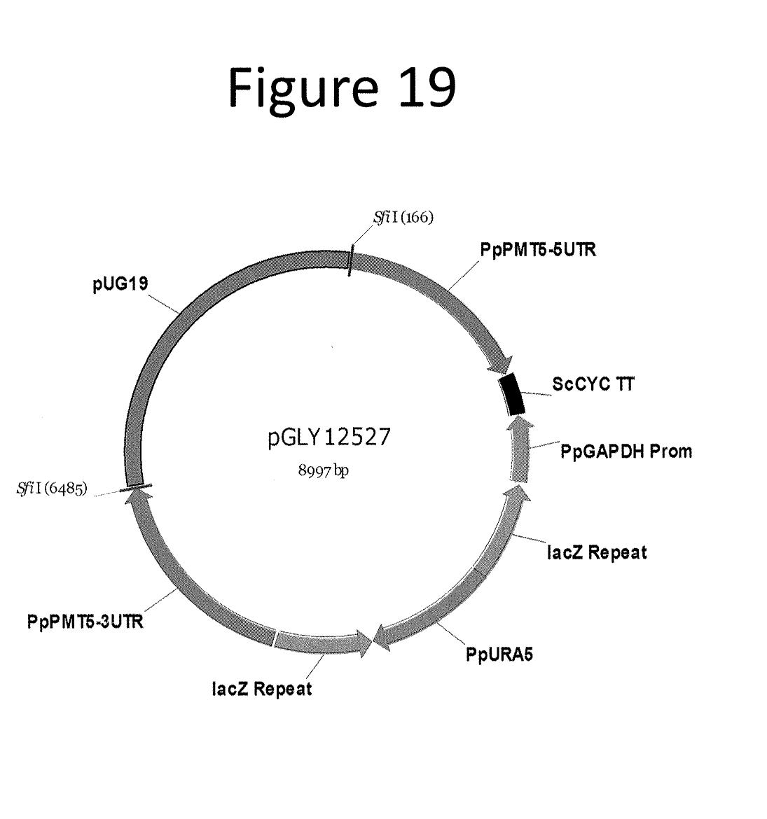

The PMT5 knock-out integration plasmid pGLY12527 (FIG. 19) was linearized with SfiI and the linearized plasmid was transformed into the 5-FOA counter selected YGLY28423 Pichia pastoris strain YGLY30398 (i.e., ura5 deletion in strain YGLY28423, FIG. 14), to produce och1, pmt5, strain YGLY32107.

The genomic integration of pGLY12527 at the PMT5 locus was confirmed by cPCR using the primers, PpPMT5-A (5′-TGTCAATCAATAAGTGTGGCAAATGCG-3′) (SEQ ID NO: 19) and ScCYCTT-RV (5′-GCGGATCCAGCTTGCAAATT-3′) (SEQ ID NO: 20) or PpPMT5-B (5′-GGGGAAAATGTACAAGGTGTAGTATCCAG-3′) (SEQ ID NO: 21) and PpURA5-FW (5′-TTTCTTCTGTTTCGGAGCTTTGG-3) (SEQ ID NO: 22). Loss of genomic PMT5 sequences was confirmed using cPCR primers, PpPMT5-C(5′-AGGTCAGTATTATAGGAGACAAAGACTATGTCCC-3′) (SEQ ID NO: 23) and PpPMT5-D (5′-CCAATAGATTGGCAAGTTACCTAACAAGTAG-3′) (SEQ ID NO: 24). The PCR conditions were one cycle of 95° C. for two minutes, 35 cycles of 95° C. for 20 seconds, 52° C. for 20 seconds, and 72° C. for two minutes; followed by one cycle of 72° C. for 10 minutes.

### Example 10

### Knockout of pmt2 Using Plasmids pGLY12535 and pGLY12536, a Split-G418 Two-Plasmid Cre/Lox Recombination System

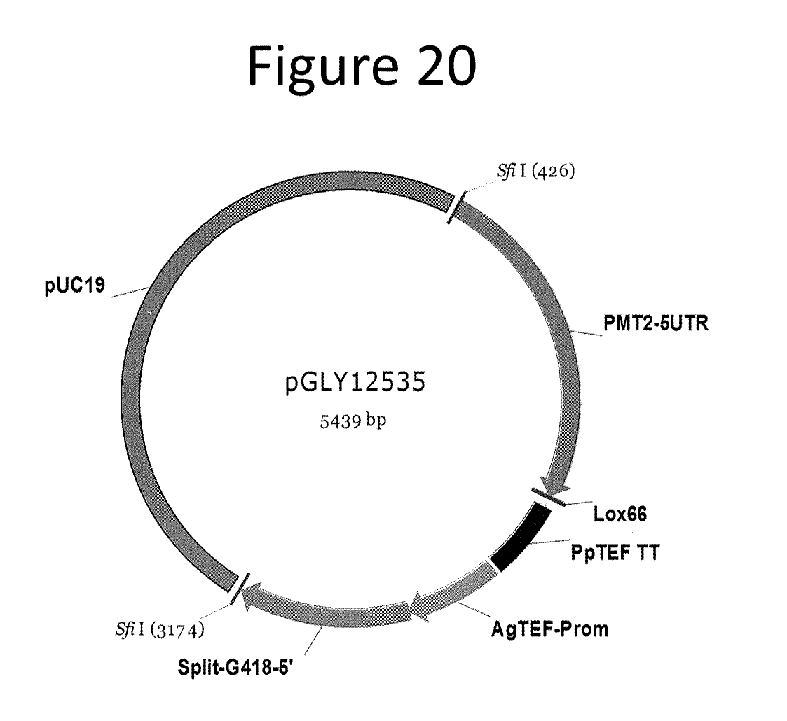

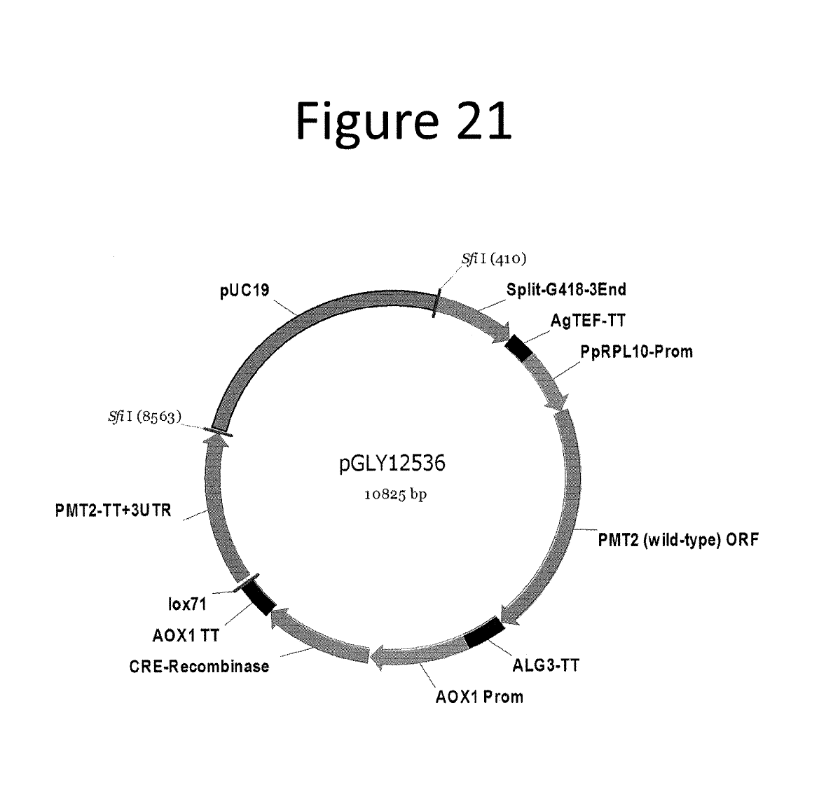

To generate a linear Cre-LoxP PMT2 DNA replacement allele, 10 ug of plasmids pGLY12535 (FIG. 20) and plasmid pGLY12536 (FIG. 21) DNA were combined into 1 tube and digested with SfiI restriction enzyme.

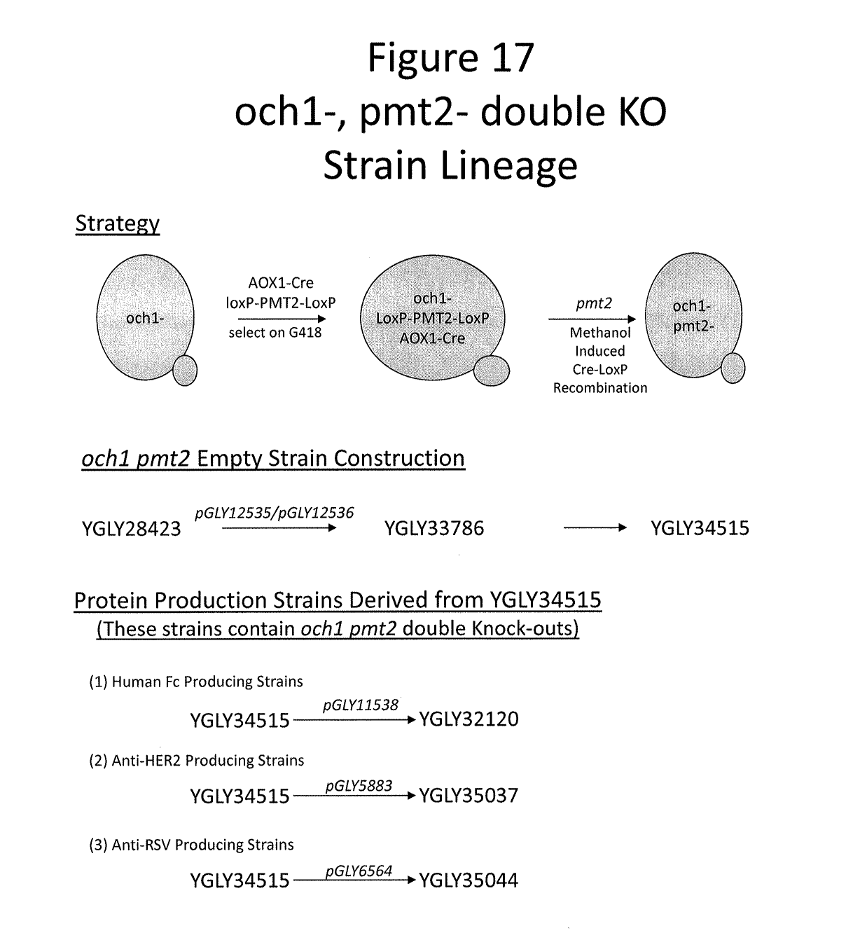

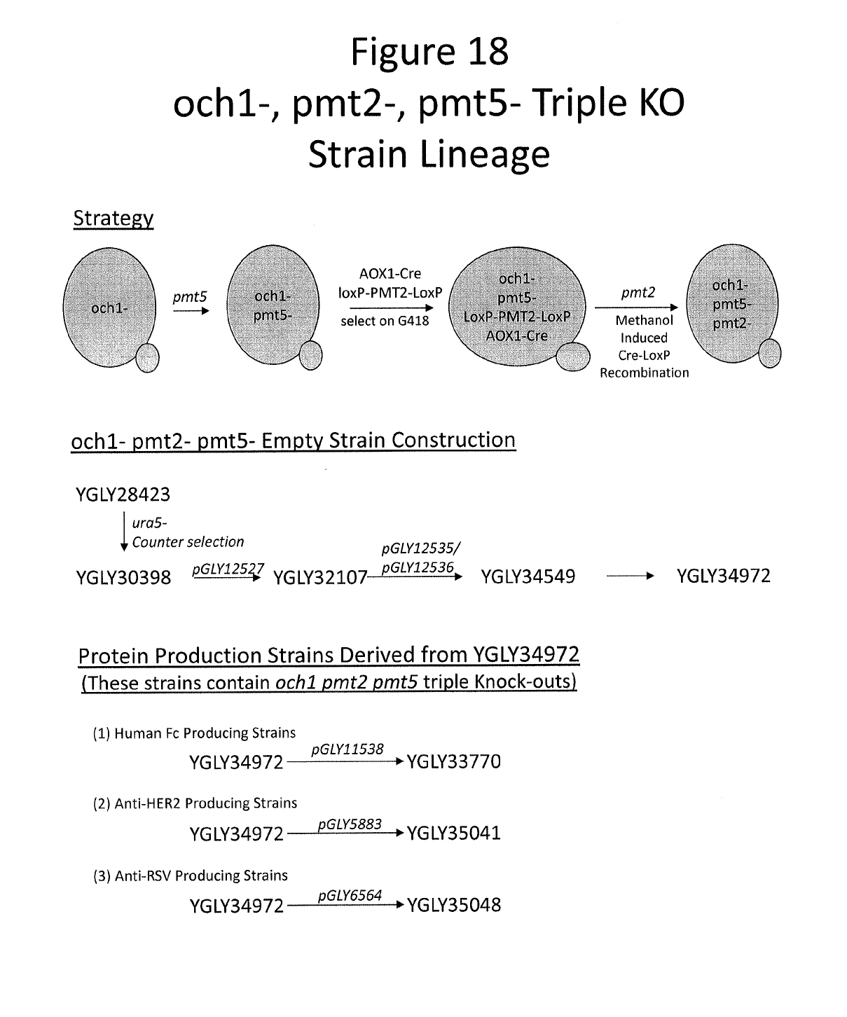

Yeast strains YGLY28423 (och1 single deletion, FIG. 17) and YGLY32107 (och1 and pmt5⁻ double deletions, FIG. 18) were used as the parental strains of the following examples. The strains were capable to produce recombinant protein with GS6.0 N-glycan structure. The SfiI digested pGLY12535 and pGLY12536 plasmid DNA was transformed into the P. pastoris strains YGLY28423 and YGLY32107 to produce PMT2 replacement strains (i.e., Cre-LoxP flanking the endogenous PMT2 locus) YGLY33786 (FIG. 17) and YGLY34549 (FIG. 18), respectively. The transformants were selected on 400 μg/mL G418 disulfate salt-YSD plates. The genomic integration at the PMT2 locus was confirmed by cPCR using the primers, PpPMT2-A (5′-AAGAAGCGTTGTAGCTGGAAGAGCA-3′) (SEQ ID NO: 25) and PpTEF-TT-RV (5′-GATAAATCGATCAAAGTTACAAACAATAACAGTAAA-3′) (SEQ ID NO: 26) or PpPMT2-B (5′-GAGTAAAACCAATTATCCCTGGGCTTTAG-3′) (SEQ ID NO: 27) and AOX1-TT-FW (5′-AAAACTATGTGGCAAGCCAAGC-3′) (SEQ ID NO: 28). The PCR conditions were one cycle of 94° C. for 30 seconds, 30 cycles of 94° C. for 20 seconds, 55° C. for 30 seconds, and 72° C. for 2 minutes; followed by one cycle of 72° C. for 5 minutes.

To induce PMT2 Knock-out using Cre-LoxP recombination, strains YGLY33786 and YGLY34549 were cultivated in the presence of methanol in 10 mL BMMY (buffered methanol complex medium, Invitrogen, a division of Life Technologies, Carlsbad, Calif.) media in a 50 mL shake flask overnight, to induce expression of the AOX1-Cre recombinase allele. Afterwards, cells were serially diluted and plated to form single colony on YSD plates. The strains YGLY34515 (och1, pmt2 double, FIG. 17) and YGLY34792 (och1, pmt2, pmt5⁻ triple, FIG. 18) were selected from the strains produced. Loss of genomic PMT2 sequences was confirmed using cPCR primers, PpPMT2-C(5′-ACGTTAAAATGAGGTTATTCAATGCCACC-3′) (SEQ ID NO: 29) and PpPMT2-D (5′-CACCGGTACCAGAATTGGATAATATTTCAA-3′) (SEQ ID NO: 30). The PCR conditions were one cycle of 94° C. for 30 seconds, 30 cycles of 94° C. for 20 seconds, 55° C. for 30 seconds, and 72° C. for 30 seconds; followed by one cycle of 72° C. for 1 minute.

### Example 11

### Engineered och1 pmt2 pmt5 Triple Knockout Strains Display an Improved Human Fc Protein Titer as Well as Reduced O-Glycan Site Occupancy Under Fermentation Conditions

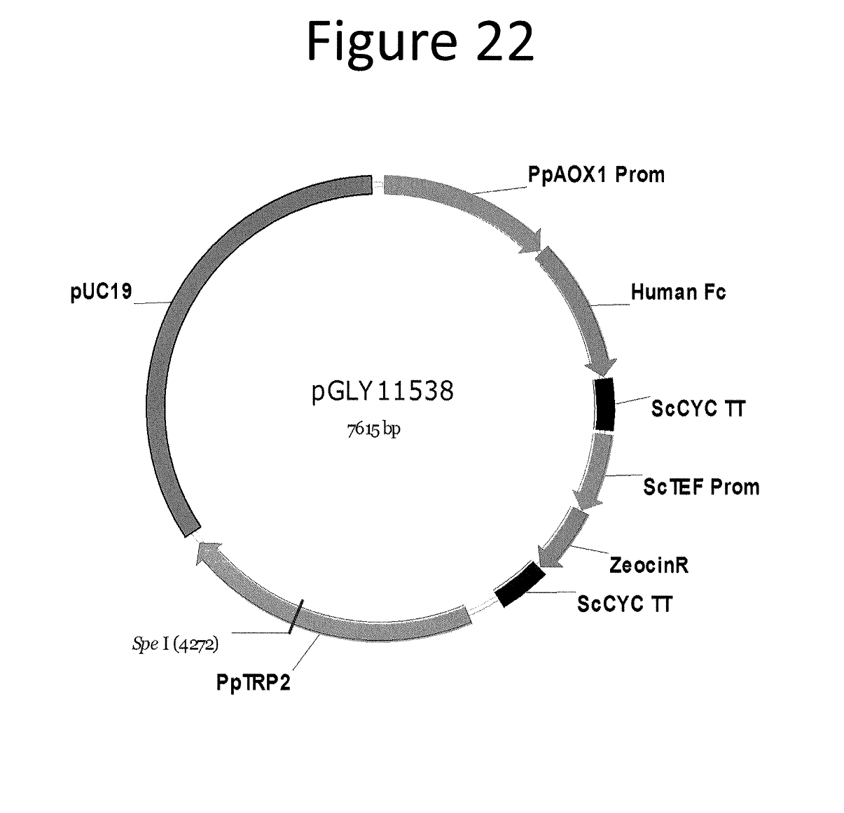

To determine whether the och1⁻, pmt2⁻, pmt5⁻ strain would have improved protein titer and reduced O-mannose site occupancy, plasmid pGLY11538 (FIG. 22), a construct containing the genes encoding the human Fc protein driven by the AOX1 promoter was introduced and selected for by resistance to Zeocin. One such human Fc expressing clone from YGLY34972, named YGLY33770, was cultivated in a 1 L fermenter and compared to (a) och1 single knockout Fc expressing strain YGLY29128 and (b) och1⁻, pmt2⁻ double knockouts Fc expressing strain YGLY32120. All runs were cultivated in the absence of chemical PMTi-4 inhibitor.

As shown in Table 6, the och1, pmt2, pmt5 knockout strain-derived clone YGLY33770 produced the highest human Fc titer with the least amount of O-linked mannose site occupancy. The YGLY33770 (och1, pmt2, pmt5) produced protein contained 0.2 mol of O-mannose per human Fc whereas the YGLY29129 (och1) and YGLY32120 (och1, pmt2) produced protein contained 3.91 and 0.24 mol of O-mannose per human Fc, respectively.

TABLE 6
Characterization of glycoengineered strain Fc expression in och1
and PMT knockout strain backgrounds. Yeast strain YGLY33770:
och1, pmt2 and pmt5 triple knock-outs. Yeast strain YGLY29189:
control strain with och1 KO. Yeast strain YGLY32120:
och1, pmt2 double knockouts.

| | O-linked Ser/Thr | Protein titer |
|---|---|---|
| Strain description | per Mab | (mg/L) |
| YGLY29128 och1 | 3.91 | 1116 |
| (control) | | |
| YGLY32120 och1, pmt2 | 0.24 | 1210 |
| (double) | | |
| YGLY33770 och1, pmt2, | 0.20 | 1299 |
| pmt5 (triple) | | |

### Example 12

### Engineered och1, pmt2, pmt5 Triple Knockout Strains Display an Improved Anti-HER2 mAb Titer, Assembly as Well as Reduced O-Glycan Site Occupancy Under Fermentation Conditions

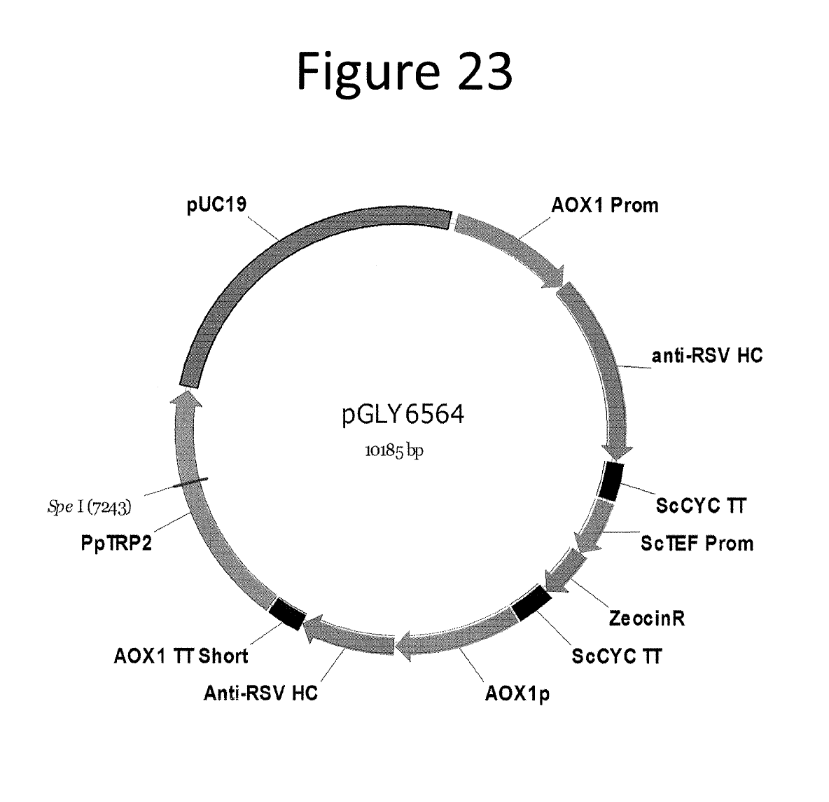

To determine whether the och1-, pmt2-, pmt5-strain would have improved mAb titer and reduced O-mannose site occupancy, plasmid pGLY5883 (FIG. 23), a construct containing the genes encoding an anti-HER2 monoclonal antibody heavy and light chains driven by the AOX1 promoters was introduced and selected for by resistance to Zeocin. One such anti-HER2 mAb expressing clone from YGLY34972, named YGLY35041, was cultivated in a 1 L fermenter and compared to (a) och1 single knockout anti-HER2 expressing strain YGLY35035 and (b) och1⁻, pmt2⁻ double knockouts anti-HER2 expressing strain YGLY35037. All runs were cultivated in the absence of chemical PMTi-4 inhibitor.

As shown in Table 7, the och1, pmt2, pmt5 knockout strain-derived clone YGLY35041 produced the highest anti-HER2 titer with the least amount of O-linked mannose site occupancy. The YGLY35041 (och1, pmt2, pmt5) produced protein contained 1.8 mol of O-mannose per anti-HER2 whereas the YGLY35035 (och1) and YGLY35037 (och1, pmt2) produced protein contained 46.1 and 2.6 mol of O-mannose per anti-HER2 mAb, respectively.

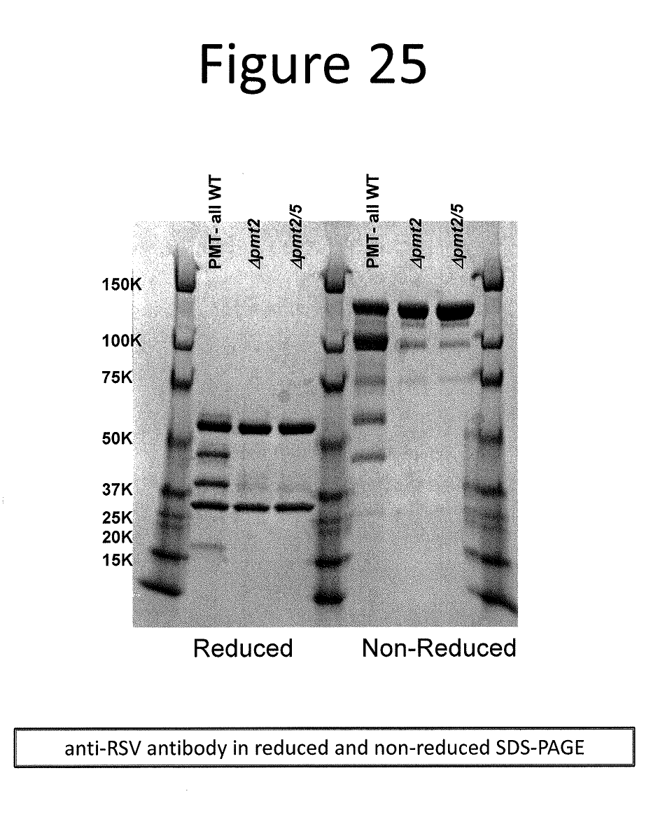

FIG. 25 shows the reducing and non-reducing SDS-PAGE for anti-HER2 material generated by och1, pmt2, pmt5 triple knockout strain YGLY35041 and its comparison with for anti-HER2 materials generated by YGLY35035 and YGLY35037. As shown in FIG. 25, the och1, pmt2, pmt5 triple knockout strain YGLY35041 produced significantly better assembled mAb than the och1 control strain YGLY35035. Moreover, the och1, pmt2, pmt5 triple knockout strain-derived material was also slightly better assembled than och1, pmt2 strain YGLY35037.

TABLE 7
Characterization of glycoengineered strain anti-HER2 expression
in och1 and PMT knockout strain backgrounds.
Yeast strain YGLY35041: och1, pmt2 and pmt5 triple knock-outs.
Yeast strain YGLY35035: control strain with och1 KO.
Yeast strain YGLY35037: och1, pmt2 double knock-outs.

| | O-linked Ser/Thr | mAb titer |
|---|---|---|
| Strain description | per Mab | (mg/L) |
| YGLY35035 och1 | 46.1 | 129 |
| (control) | | |
| YGLY35037 och1, pmt2 | 2.6 | 202 |
| (double) | | |
| YGLY35041 och1, pmt2, | 1.8 | 215 |
| pmt5 | | |
| (triple) | | |

### Example 13

### Engineered och1 pmt2 pmt5 Triple Knockout Strains Display an Improved Anti-RSV mAb Titer, Assembly as Well as Reduced O-Glycan Site Occupancy Under Fermentation Conditions

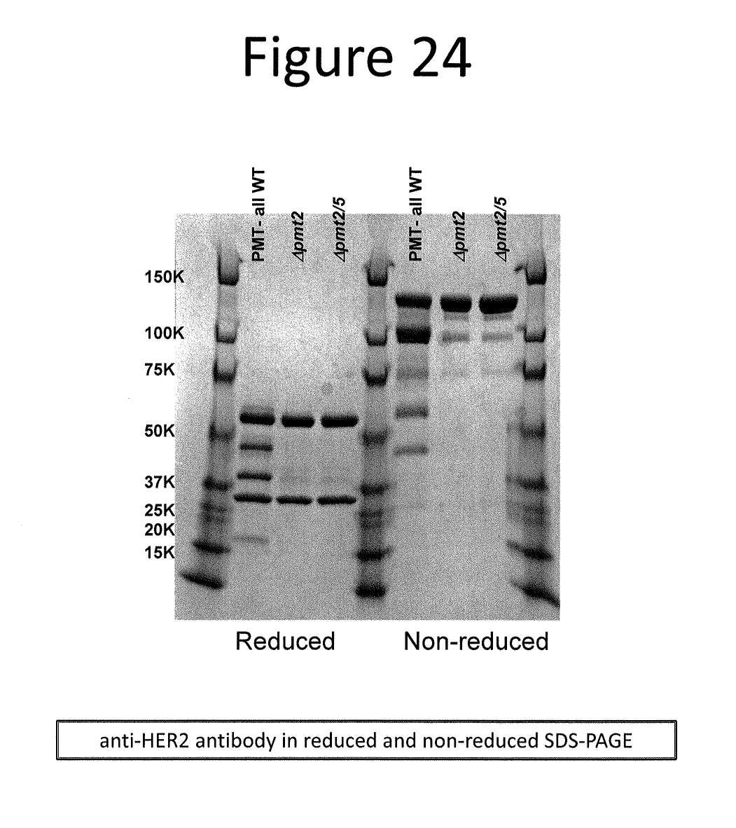

To determine whether the och1⁻, pmt2⁻, pmt5⁻ strain would have improved mAb titer and reduced O-mannose site occupancy, plasmid pGLY6564 (FIG. 24), a construct containing the genes encoding an anti-RSV monoclonal antibody heavy and light chains driven by the AOX1 promoters was introduced and selected for by resistance to Zeocin. One such anti-RSV mAb expressing clone from YGLY34972, named YGLY35048, was cultivated in a 1 L fermenter and compared to (a) och1 single knockout anti-RSV expressing strain YGLY35042 and (b) och1⁻, pmt2⁻ double knockouts anti-RSV expressing strain YGLY35044. All runs were cultivated in the absence of chemical PMTi-4 inhibitor.

As shown in Table 8, the och1, pmt2, pmt5 knockout strain-derived clone YGLY35048 produced the highest anti-RSV titer with the least amount of O-linked mannose site occupancy. The YGLY35048 (och1, pmt2, pmt5) produced protein contained 2.0 mol of O-mannose per anti-RSV whereas the YGLY35042 (och1) and YGLY35044 (och1, pmt2) produced protein contained 20.4 and 2.1 mol of O-mannose per anti-RSV mAb, respectively.

FIG. 26 shows the reducing and non-reducing SDS-PAGE for anti-RSV material generated by och1, pmt2, pmt5 triple knockout strain YGLY35048 and its comparison with for anti-RSV materials generated by YGLY35042 and YGLY35044. As shown in FIG. 26, the och1, pmt2, pmt5 triple knockout strain YGLY35048 produced significantly better assembled mAb than the och1 control strain YGLY35042. Moreover, the och1, pmt2, pmt5 triple knockout strain-derived material was also slightly better assembled than och1, pmt2 strain YGLY35044.

TABLE 8
Characterization of glycoengineered strain anti-RSV expression
in och1 and PMT knockout strain backgrounds.
Yeast strain YGLY35048: och1, pmt2 and pmt5 triple knock-outs.
Yeast strain YGLY35042: control strain with och1 KO.
Yeast strain YGLY35044: och1, pmt2 double knockouts.

| | O-linked Ser/Thr | mAb titer |
|---|---|---|
| Strain description | per Mab | (mg/L) |
| YGLY35042 och1 | 20.4 | 97 |
| (control) | | |
| YGLY35044 och1, pmt2 | 2.1 | 474 |
| (double) | | |
| YGLY35048 och1, pmt2, | 2.0 | 573 |
| pmt5 | | |
| (triple) | | |

### REFERENCES

Barnard G C, Kull A R, Sharkey N S, Shaikh S S, Rittenhour A M, Burnina I, Jiang Y, Li F, Lynaugh H, Mitchell T, Nett J H, Nylen A, Potgieter T I, Prinz B, Rios S E, Zha D, Sethuraman N, Stadheim T A, Bobrowicz P. High-throughput screening and selection of yeast cell lines expressing monoclonal antibodies. J Ind Microbiol Biotechnol. 2010 September; 37(9):961-71. Epub 2010 Aug. 15. PubMed PMID: 20711797.
Kuroda K, Kobayashi K, Kitagawa Y, Nakagawa T, Tsumura H, Komeda T, Shinmi D, Mori E, Motoki K, Fuju K, Sakai T, Nonaka K, Suzuki T, Ichikawa K, Chiba Y, Jigami Y. Efficient antibody production upon suppression of O mannosylation in the yeast Ogataea minuta. Appl Environ Microbiol. 2008 January; 74(2):446-53. Epub 2007 Nov. 26. PubMed PMID: 18039826; PubMed Central PMCID: PMC2223252.
Nett J H, Gerngross T U. Cloning and disruption of the PpURA5 gene and construction of a set of integration vectors for the stable genetic modification of Pichia pastoris. Yeast. 2003 November; 20(15):1279-90.
Stadheim T A, Li H, Kett W, Burnina I N, Gerngross T U. Use of high-performance anion exchange chromatography with pulsed amperometric detection for O-glycan determination in yeast. Nat Protoc. 2008; 3(6):1026-31. PubMed PMID: 18546597.
Tanner et al. in International Published Patent Application No. PCT WO 94/04687: “Modified Fungal Cells and Method for Producing Recombinant Products”.
Ng et al. in U.S. Published Patent Application No. US 2002/0068325 A1: “Methods and Compositions For Highly Efficient Production of Heterologous Proteins In Yeast”
Desai et al., U.S. Published Patent Application No. US 2011/0076721 A1: “Efficient Production of Heterologous Proteins Using Mannosyl Transferase Inhibitors”.
Papac D I, Briggs J B, Chin E T, Jones A J. A high-throughput microscale method to release N-linked oligosaccharides from glycoproteins for matrix-assisted laser desorption/ionization time-of-flight mass spectrometric analysis. Glycobiology. 1998 May; 8(5):445-54. PubMed PMID: 9597542.
Willger S D, Ernst J F, Alspaugh J A, Lengeler K B. Characterization of the PMT gene family in Cryptococcus neoformans. PLoS One. 2009 Jul. 27; 4(7):e6321. PubMed PMID: 19633715
Goto M. Protein O-glycosylation in fungi: diverse structures and multiple functions. Biosci Biotechnol Biochem. 2007 June; 71(6):1415-27. Review. PubMed PMID: 17587671.
Górka-Nieć W, Bańkowska R, Palamarczyk G, Krotkiewski H, Kruszewska J S. Protein glycosylation in pmt mutants of Saccharomyces cerevisiae. Influence of heterologously expressed cellobiohydrolase II of Trichoderma reesei and elevated levels of GDP-mannose and cis-prenyltransferase activity. Biochim Biophys Acta. 2007 May; 1770(5):774-80. Epub 2007 Feb. 1. PubMed PMID: 17343985.
Prill S K, Klinkert B, Timpel C, Gale C A, Schröppel K, Ernst J F. PMT family of Candida albicans: five protein mannosyltransferase isoforms affect growth, morphogenesis and antifungal resistance. Mol Microbiol. 2005 January; 55(2):546-60. PubMed PMID: 15659169.
Strahl-Bolsinger S, Gentzsch M, Tanner W. Protein O-mannosylation. Biochim Biophys Acta. 1999 January 6; 1426(2):297-307. Review. PubMed PMID: 9878797.
Gentzsch M, Tanner W. The PMT gene family: protein O-glycosylation in Saccharomyces cerevisiae is vital. EMBO J. 1996 Nov. 1; 15(21):5752-9. PubMed PMID: 8918452; PubMed Central PMCID: PMC452322.
Gentzsch M, Tanner W. Protein-O-glycosylation in yeast: protein-specific mannosyltransferases. Glycobiology. 1997 June; 7(4):481-6. PubMed PMID: 9184828.
Ernst J F, Prill S K. O-glycosylation. Med Mycol. 2001; 39 Suppl 1:67-74. Review. PubMed PMID: 11800270.
Lommel M, Strahl S. Protein O-mannosylation: conserved from bacteria to humans. Glycobiology. 2009 August; 19(8):816-28. Epub 2009 May 9. Review. PubMed PMID: 19429925.
Jiang Y, Li F, Zha D, Potgieter T I, Mitchell T, Moore R, Cukan M, Houston-Cummings N R, Nylen A, Drummond J E, McKelvey T W, d'Anjou M, Stadheim T A, Sethuraman N, Li H. Purification process development of a recombinant monoclonal antibody expressed in glycoengineered Pichia pastoris. Protein Expr Purif. 2011 March; 76(1):7-14. Epub 2010 Nov. 11. PubMed PMID: 21074617.
Hamilton S R, Davidson R C, Sethuraman N, Nett J H, Jiang Y, Rios S, Bobrowicz P, Stadheim T A, Li H, Choi B K, Hopkins D, Wischnewski H, Roser J, Mitchell T, Strawbridge R R, Hoopes J, Wildt S, Gerngross T U. Humanization of yeast to produce complex terminally sialylated glycoproteins. Science. 2006 Sep. 8; 313(5792):1441-3. PubMed PMID: 16960007.
Wildt S, Gerngross TU. The humanization of N-glycosylation pathways in yeast. Nat Rev Microbiol. 2005 February; 3(2):119-28. Review. PubMed PMID: 15685223.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

1. An isolated fungal or lower eukaryotic host cell wherein said cell does not express functional PMT2 polypeptide, does not express functional OCH1 polypeptide; and, optionally, does not express functional PNT5 polypeptide.

2. The isolated fungal or lower eukaryotic host cell of claim 1 which is a Pichia cell.

3. The isolated fungal or lower eukaryotic host cell of claim 1 wherein endogenous PMT2 polynucleotide and/or endogenous OCH1 polynucleotide; and/or endogenous PMT5 polynucleotide is partially deleted, fully deleted, point mutated or disrupted.

4. The isolated fungal or lower eukaryotic host cell of claim 1 having a cell wall with an average N-glycan mannose content of about 3-10 mannose residues per N-glycan on said cell wall.

5. The isolated fungal or lower eukaryotic host cell of claim 1 which comprises a heterologous immunoglobulin polypeptide.

6. A culture medium comprising the isolated fungal or lower eukaryotic host cell of claim 1.

7. The isolated fungal or lower eukaryotic host cell of claim 1 which is a Pichia pastoris cell.

8. A method for producing an isolated pmt2-, och1- or pmt2-, och1-, pmt5-fungal or lower eukaryotic host cell comprising expressing a site-specific recombinase in an och1- or och1-, pmt5-fungal or lower eukaryotic host cell; wherein site-specific recombinase target sequences are at the 5′ and 3′ side of endogenous chromosomal PMT2 in the cell; and wherein, the recombinase, when expressed in the cell, recombines the target sequences such that the PMT2 sequence between the target sequences is deleted from the chromosome.

9. The method of claim 8 wherein the fungal or lower eukaryotic host cell has a cell wall with an average N-glycan mannose content of about 3-10 mannose residues per N-glycan on said cell wall.

10. The method of claim 8 wherein the site-specific recombinase is Cre and the site-specific recombinase target sequences are loxP sites.

11. An isolated fungal or lower eukaryotic host cell produced by the method of claim 8.

12. The isolated fungal or lower eukaryotic host cell of claim 11 which is a Pichia cell.

13. A method for producing an isolated pmt2-, och1- or pmt2-, och1-, pmt5-fungal or lower eukaryotic host cell comprising deleting endogenous PMT2 in an och1- or och1-, pmt5-fungal or lower eukaryotic host cell that comprises PMT2 operably linked to an inducible promoter under conditions whereby the promoter is induced and then, optionally, culturing the cell under conditions whereby the promoter is not induced.

14. The method of claim 13 wherein the promoter is an AOX1 promoter and the conditions whereby the promoter is induced comprising culturing the cell in the presence of methanol.

15. The method of claim 13 wherein the fungal or lower eukaryotic host cell has a cell wall with an average N-glycan mannose content of about 3-10 mannose residues per N-glycan on said cell wall.

16. An isolated fungal or lower eukaryotic host cell produced by the method of claim 13.

17. The isolated fungal or lower eukaryotic host cell of claim 16 which is a Pichia cell.

18. The isolated fungal or lower eukaryotic host cell of claim 1 comprising one or more of the following characteristics:
(i) wherein one or more endogenous beta-mannosyltransferase genes are mutated;
(ii) comprising a polynucleotide encoding an alpha-1,2 mannosidase enzyme;
(iii) wherein one or more endogenous phosphomannosyl transferases are mutated, disrupted, truncated or partially or fully deleted;
(iv) comprising a Leishmania sp. single-subunit oligosaccharyltransferase;
(v) wherein endogenous Alg3 is mutated, disrupted, truncated or partially or fully deleted;
(vi) comprising a polynucleotide encoding an endomannosidase;
(vii) comprising one or more polynucleotides encoding a bifunctional UDP-N-acetylglucosamine-2-epimerase/N-acetylmannosamine kinase, an N-acetylneuraminate-9-phosphate synthase, or a CMP-sialic acid synthase;
(viii) wherein endogenous ATT1 gene is mutated, disrupted, truncated or partially or fully deleted;
(ix) wherein endogenous OCH1 is mutated, disrupted, truncated or partially or fully deleted;
(x) comprising a polynucleotide encoding galactosyltransferase;
(xi) comprising a polynucleotide encoding nucleotide sugar transporter;
(xii) comprising a polynucleotide encoding sialyltransferase; and/or
(xiii) comprising a polynucleotide encoding acetylglucosaminyl transferase.

19. A method for producing a heterologous polypeptide comprising introducing, into said cell of claim 1, a polynucleotide encoding the heterologous polypeptide and culturing the host cell comprising the polynucleotide encoding the heterologous polypeptide under conditions allowing expression of the heterologous polypeptide.

20. The method of claim 18 further comprising isolating the heterologous polypeptide from the cells and/or culture medium in which the cells are cultured.

21. The method of claim 18 wherein the heterologous polypeptide is an immunoglobulin.
